(19) United States
(12) Patent Application Publication
(10) Pub. No.: US 2026/0083579 A1
(43) Pub. Date: Mar. 26, 2026

(54) Mechanical Crimper

(71) Applicant: St. Jude Medical, Cardiology Division, Inc., St. Paul, MN (US)

(72) Inventors: William H. Peckels, Robbinsdale, MN (US); Keith High, White Bear Lake, MN (US); Philip Osterbauer, Wyoming, MN (US); Michael Shane Morrissey, St. Paul, MN (US)

(73) Assignee: St. Jude Medical, Cardiology Division, Inc., St. Paul, MN (US)

(21) Appl. No.: 19/333,730

(22) Filed: Sep. 19, 2025

Related U.S. Application Data
(60) Provisional application No. 63/697,858, filed on Sep. 23, 2024.

(51) Int. Cl. A61F 2/95

(52) U.S. Cl. CPC A61F 2/9524

(57) ABSTRACT

A crimping device for crimping a medical device includes a housing assembly, an iris assembly including crimping members, a handle assembly, and a rotating body operably coupled to the handle and to the crimping members such that rotation of the handle causes rotation of the rotating body to drive the crimping members toward or away from each other to close or open the iris assembly. A sizing key is configured to be coupled to the rotating body. When the sizing key is not coupled to the rotating body, the rotating body has a first range of rotation corresponding to a first minimum size of the iris assembly. When the sizing key is coupled to the rotating body, the rotating body has a second range of rotation corresponding a second minimum size of the iris assembly, which is larger than the first minimum size of the iris assembly.

### CROSS-REFERENCE TO RELATED APPLICATIONS

This application claims the benefit of priority to U.S. Provisional Patent Application No. 63/697,858, filed Sep. 23, 2024, the disclosure of which is hereby incorporated by reference herein.

### BACKGROUND OF THE DISCLOSURE

Valvular heart disease, and specifically aortic and mitral valve disease, is a significant health issue in the United States. Valve replacement is one option for treating heart valve diseases. Prosthetic heart valves include surgical heart valves, as well as collapsible and expandable heart valves intended for transcatheter aortic valve replacement or implantation (“TAVR” or “TAVI”) or transcatheter mitral valve replacement (“TMVR”). Surgical or mechanical heart valves may be sutured into a native annulus of a patient during an open-heart surgical procedure, for example. Collapsible and expandable heart valves may be delivered into a patient via a delivery apparatus such as a catheter to avoid a more invasive procedure such as full open-chest, open-heart surgery. As used herein, reference to a “collapsible and expandable” heart valve includes heart valves that are formed with a small cross-section that enables them to be delivered into a patient through a catheter in a minimally invasive procedure, and then expanded to an operable state once in place, as well as heart valves that, after construction, are first collapsed to a small cross-section for delivery into a patient and then expanded to an operable size once in place in the valve annulus.

Collapsible and expandable prosthetic heart valves typically take the form of a one-way valve structure (often referred to as a valve assembly) mounted within an expandable frame (the terms “stent” and “frame” may be used interchangeably herein). In general, these collapsible and expandable heart valves include a self-expanding, mechanically-expandable, or balloon-expandable frame, often made of nitinol or another shape-memory metal or metal alloy (for self-expanding frames) or steel or cobalt chromium (for balloon-expandable frames). The one-way valve assembly mounted to/within the stent includes one or more leaflets and may also include a cuff or skirt. The cuff may be disposed on the stent's interior or luminal surface, its exterior or abluminal surface, and/or on both surfaces. A cuff helps to ensure that blood does not just flow around the valve leaflets if the valve or valve assembly is not optimally seated in a valve annulus. A cuff, or a portion of a cuff disposed on the exterior of the stent, can help prevent leakage around the outside of the valve (the latter known as paravalvular or “PV” leakage).

Balloon expandable valves are typically delivered to the native annulus while collapsed (or “crimped”) onto a deflated balloon of a balloon catheter, with the collapsed valve being either covered or uncovered by an overlying sheath. Once the crimped prosthetic heart valve is positioned within the annulus of the native heart valve that is being replaced, the balloon is inflated to force the balloon-expandable valve to transition from the collapsed or crimped condition into an expanded or deployed condition, with the prosthetic heart valve tending to remain in the shape into which it is expanded by the balloon. Typically, when the position of the collapsed prosthetic heart valve is determined to be in the desired position relative to the native annulus (e.g. via visualization under fluoroscopy), a fluid (typically a liquid although gas could be used as well) such as saline is pushed via a syringe (manually, automatically, or semi-automatically) through the balloon catheter to cause the balloon to begin to fill and expand, and thus cause the overlying prosthetic heart valve to expand into the native annulus.

### SUMMARY OF THE DISCLOSURE

According to an aspect of the disclosure, a crimping device includes a first pinion gear, a handle operably attached to the first pinion gear so that rotation of the handle in a first rotational direction causes rotation of the first pinion gear in the first rotational direction, and a second gear having gear teeth operably engaged with gear teeth of the first pinion gear so that rotation of the first pinion gear in the first rotational direction causes rotation of the second gear in a second rotational direction opposite the first rotational direction. The second gear may have a diameter that is larger than a diameter of the first pinion gear to provide mechanical advantage. A plurality of contact members may each have a contact arm so that the plurality of contact arms collectively form an iris opening having a central longitudinal axis. Rotation of the second gear may move the plurality of contact arms toward or away from the central longitudinal axis to change a size of the iris opening. A housing assembly may include a base which may have a bottom surface configured to be placed on a flat surface, and a top surface opposite the bottom surface. A longitudinal center of the first pinion gear may be positioned a first distance above the top surface of the base, and the central longitudinal axis of the iris opening may be positioned a second distance above the top surface of the base. The first distance may be greater than the second distance. The handle may have a first handle end with a center that is coaxial with the longitudinal center of the first pinion gear, a second handle with a grip, and a handle body extending between the first handle end and the second handle end. A maximum length between the center of the first handle end and an outer surface of the second handle end may be smaller than the first distance. The maximum length between the center of the first handle end and the outer surface of the second handle end may be smaller than the first distance by at least 5 cm. A pinion drive shaft may be received within the first pinion gear, and the pinion drive shaft may also be received within the first handle end, the first handle end being rotationally fixed to the pinion drive shaft. The handle may be removably coupled to the pinion drive shaft. The pinion drive shaft may be parallel to the central longitudinal axis of the iris opening. During rotation of the handle through a 360 degree arc around the pinion drive shaft, no portion of the handle may enter a zone of visual interference, the zone of visual interference being defined by an imaginary cylinder having a radius of about 25 mm and being coaxial with the longitudinal axis of the iris assembly. During rotation of the handle through a 360 degree arc around the pinion drive shaft, no portion of the handle may be positioned closer than about 5 cm from the top surface of the base.

The diameter of the second gear may be between about two times and about three times larger than the diameter of the first pinion gear. The base may have a length dimension and a width dimension smaller than the length dimension, the width dimension being defined between a first side edge and a second side edge of the base. The housing assembly may include a shell in which the first pinion gear and second gear are received, and the housing assembly may have an assembled condition in which the shell is coupled to the base such that a distance between a rear surface of the shell and the first side edge of the base is smaller than a distance between a front surface of the shell and the second side edge of the base. The handle may be positioned closer to the front surface of the shell than the second surface of the shell. A spring pin may have (i) a pin portion configured to contact a circular rim of the first pinion gear and (ii) a biasing member that applies force on the pin portion toward the circular rim. The first pinion gear may include a pin detent that interrupts the circular rim, the pin detent sized and shaped to receive the pin portion of the spring pin such that, when the pin portion of the spring pin is received within the pin detent, the pinion gear is locked from rotating. The pin detent may be positioned so that, when the spring detent is aligned with the spring pin so that the pin portion of the spring pin is received within the pin detent, the size of the iris opening is at a predetermined minimum size. The handle may be rotatable along a circular path having an upstroke in which a grip end of the handle is moving away from the base, and a downstroke in which the grip end of the handle is moving toward the base. The handle, the first pinion gear, and the second gear may be configured so that, as the handle rotates to reduce the size of the iris opening, the handle is rotating along the downstroke as the size of the iris opening reaches the predetermined minimum size.

The diameter of the second gear may be between about two times and about three times larger than the diameter of the first pinion gear. The base may have a length dimension and a width dimension smaller than the length dimension, the width dimension being defined between a first side edge and a second side edge of the base. The housing assembly may include a shell in which the first pinion gear and second gear are received, and the housing assembly may have an assembled condition in which the shell is coupled to the base such that a distance between a rear surface of the shell and the first side edge of the base is smaller than a distance between a front surface of the shell and the second side edge of the base. The handle may be positioned closer to the front surface of the shell than the second surface of the shell. A spring pin may have (i) a pin portion configured to contact a circular rim of the first pinion gear and (ii) a biasing member that applies force on the pin portion toward the circular rim. The first pinion gear may include a pin detent that interrupts the circular rim, the pin detent sized and shaped to receive the pin portion of the spring pin such that, when the pin portion of the spring pin is received within the pin detent, the pinion gear is locked from rotating. The pin detent may be positioned so that, when the spring detent is aligned with the spring pin so that the pin portion of the spring pin is received within the pin detent, the size of the iris opening is at a predetermined minimum size. The handle may be rotatable along a circular path having an upstroke in which a grip end of the handle is moving away from the base, and a downstroke in which the grip end of the handle is moving toward the base. The handle, the first pinion gear, and the second gear may be configured so that, as the handle rotates to reduce the size of the iris opening, the handle is rotating along the downstroke as the size of the iris opening reaches the predetermined minimum size. A first crimping guide may be fixed to the housing assembly, the first crimping device having a plurality of radially extending slots, the plurality of radially extending slots each extending in a direction toward and away from the central longitudinal axis of the iris opening. Each of the plurality of contact members may include a protrusion received within a corresponding one of the radially extending slots of the first crimping guide. A second crimping guide may be fixed to the second gear, the second crimping guide having a plurality of grooves having a spiral shape that spiral around the central longitudinal axis of the iris opening, each of the plurality of protrusions being received within one of the plurality of grooves.

According to another aspect of the disclosure, a crimping device is for crimping a medical device from a first larger diameter to a second smaller diameter. The crimping device includes a housing assembly, an iris assembly including a plurality of crimping members, a handle assembly operably coupled to the housing assembly, and a rotating body operably coupled to the handle and to the plurality of crimping members such that rotation of the handle causes rotation of the rotating body, and rotation of the rotating body drives the crimping members toward or away from each other to close or open the iris assembly, respectively. The crimping device also includes a sizing key configured to be coupled to the rotating body. When the sizing key is not coupled to the rotating body, the rotating body has a first range of rotation corresponding to a first minimum size of the iris assembly. When the sizing key is coupled to the rotating body, the rotating body has a second range of rotation corresponding a second minimum size of the iris assembly. The second minimum size of the iris assembly is larger than the first minimum size of the iris assembly. The sizing key may include a protrusion that extends radially outward from the rotating body when the sizing key is coupled to the rotating body. The crimping device may also include a holding mechanism, the holding mechanism including a fixed member secured to the housing assembly, the fixed member defining a stop surface, whereby during rotation of the handle and rotation of the rotating body, while the sizing key is coupled to the rotating body, the protrusion of the sizing key is configured to abut the stop surface of the fixed member when the iris assembly has the second minimum size to limit the iris assembly from a reduction in size beyond the second minimum size. The holding mechanism may include a pivoting member defining a holding surface so that, while the sizing key is coupled to the rotating body, the protrusion of the sizing key is configured to abut the holding surface of the pivoting member when the iris assembly has the second minimum size such that the protrusion of the sizing key is positioned between the stop surface and the holding surface. While the sizing key is coupled to the rotating body, and while the protrusion of the sizing key is positioned between the stop surface and the holding surface, the rotating body may be prevented from rotating to maintain the iris assembly at the second minimum size. The holding mechanism may include a release button accessible through the housing assembly, whereby while the sizing key is coupled to the rotating body, and while the protrusion of the sizing key is positioned between the stop surface and the holding surface, depressing the release button may move the pivoting member so that the holding surface is no longer in abutment with the protrusion of the sizing key. The release button may include a first extension in contact with the pivoting member, and a second extension in contact with the fixed member. The pivoting member may be configured to rotate about a first rotational axis, and a first biasing member that is contact with both the pivoting member and the fixed member may bias the pivoting member to rotate in a first direction about the first rotational axis. Upon being depressed, the release button may cause the pivoting member to rotate in a second direction opposite the first direction about the first rotational axis while compressing the first biasing member. A second biasing member may be in contact with both the second extension of the release button and the fixing member, the second biasing member configured to move the release button from the depressed condition to a non-depressed condition.

The rotating body may include a first shell coupled to a second shell. The handle assembly may include a first arm and a second arm, the first arm being attached to the first shell and the second arm being attached to the second shell. The handle assembly may include a first arm and a second arm, the first arm being integral with the first shell, the second arm being integral with the second shell. The first arm and the second arm may each have a free end, and a grip member may extend between, and may be coupled to, the free end of the first arm and the free end of the second arm. The handle assembly may be rotatable about a handle axis that is coaxial with the iris assembly. A retainer may be configured to be releasably coupled to the housing assembly, the retainer including a groove to receive a catheter of a delivery device, the groove being coaxial with the iris assembly when the retainer is coupled to the housing assembly. The retainer may be formed of a single integral member. The groove of the retainer may be open so as to form a portion of a cylindrical surface. The retainer may be formed of two pieces configured to clamp together in an assembled condition. In the assembled condition of the retainer, the groove of the retainer may be closed so as to form a fully cylindrical surface.

According to another aspect of the disclosure, a method of crimping a prosthetic heart valve includes attaching a sizing key to a rotating body of a crimper device, positioning the prosthetic heart valve within an open iris assembly of the crimper device, and rotating a handle of the crimper device in a first direction to close the iris assembly to a prescribed minimum size over the prosthetic heart valve, until the sizing key abuts a stop surface of a holding member of the crimping device to prevent further rotation of the handle in the first direction. The method includes waiting a prescribed amount of time while the iris assembly has the prescribed minimum size. After waiting the prescribed amount of time, the handle of the crimper device is rotated in a second direction opposite the first direction to open the iris assembly. The crimped prosthetic heart valve is removed from the iris assembly. When the iris assembly has the prescribed minimum size, the sizing key may abut a holding surface of the holding member to prevent rotation of the handle in the second direction. A release button of the holding member may be depressed after waiting the prescribed amount of time and prior to rotating the handle of the crimper device in the second direction. Prior to attaching the sizing key to the rotating body, the rotating key may be removed from packaging containing (i) the prosthetic heart valve and/or (ii) a delivery device for delivering the prosthetic heart valve. The sizing key may have a configuration to prevent the iris assembly from reducing in size beyond the prescribed minimum size when the sizing key is coupled to the rotating body, the prescribed minimum size corresponding to a desired crimp size for the prosthetic heart valve. A retainer may be coupled to a catheter of a delivery device for delivering the prosthetic heart valve prior to rotating the handle of the crimper. Coupling the retainer to the catheter may include snapping the catheter into a groove of the retainer. Coupling the retainer to the catheter may include clamping two pieces of the retainer over the catheter. The retainer may be coupled to the housing assembly prior to rotating the handle of the crimper. During rotating the handle of the crimper, the retainer may maintain the catheter of the delivery device along a central longitudinal axis of the iris assembly.

According to another aspect of the disclosure, a crimping device is for crimping a medical device from a first larger diameter to a second smaller diameter. The crimping device includes an iris assembly including a plurality of crimping members. A first crimping guide is positioned adjacent to the plurality of crimping members, the first crimping guide including a plurality of first radial slots therein, each of the plurality of crimping members having a first pin extending through a corresponding one of the first radial slots. A first cam track is positioned adjacent to the first crimping guide so that the first crimping guide is positioned between the plurality of crimping members and the first cam track, the first cam track including a plurality of first spiral grooves, the first pin of each of the plurality of crimping members extending into a corresponding one of the plurality of first spiral grooves. The handle assembly is operably coupled to the first crimping guide such that, upon rotation of the handle assembly, the first crimping guide and the plurality of crimping members rotate relative to a central longitudinal axis of the iris assembly, while the first cam track remains stationary relative to the central longitudinal axis of the iris assembly. A second crimping guide may be positioned adjacent to the plurality of crimping members, the second crimping guide including a plurality of second radial slots therein, each of the plurality of crimping members having a second pin extending through a corresponding one of the second radial slots. The first and second crimping guides may encase the plurality of crimping members. A second cam track may be positioned adjacent to the second crimping guide so that the second crimping guide is positioned between the plurality of crimping members and the second cam track, the second cam track including a plurality of second spiral grooves, the second pin of each of the plurality of crimping members extending into a corresponding one of the plurality of second spiral grooves. The first crimping guide may define a central opening, the first cam track may define a central opening, and the handle assembly may define a central opening, the central openings of the first crimping guide, the first cam track, and the handle assembly may align with the central longitudinal axis of the iris assembly. The first crimping guide may include a rim adjacent the central opening of the first crimping guide, the rim including a plurality of protrusions extending through the central opening of the first cam track and into the central opening of the handle assembly. The central opening of the handle assembly may include a plurality of recesses that receive corresponding ones of the plurality of protrusion of the rim of the first crimping guide so that torque on the handle assembly is transmissible to the first crimping guide.

According to a further aspect of the disclosure, a method of crimping a prosthetic heart valve includes positioning the prosthetic heart valve within an open iris assembly of a crimper device while the prosthetic heart valve is positioned over a balloon of a balloon catheter of a delivery device. While the prosthetic heart valve is positioned within the open iris assembly, a handle of the crimper device is rotated in a first direction to close the iris assembly to crimp the prosthetic heart valve onto the balloon. While rotating the handle of the crimper device, a plurality of crimping members that form the iris assembly rotate around a longitudinal axis of the iris assembly while the crimping members draw closer to each other to close the iris assembly. While the plurality of crimping members rotate around the longitudinal axis of the iris assembly, the plurality of crimping members may contact the prosthetic heart valve to force the prosthetic heart valve to rotate around the longitudinal axis of the iris assembly and relative to the balloon. While the prosthetic heart valve rotates relative to the balloon, prosthetic leaflets of the prosthetic heart valve may take a spiral configuration as the prosthetic heart valve crimps.

### BRIEF DESCRIPTION OF THE DRAWINGS

FIG. 1 is a perspective view of an example of a prosthetic heart valve.

FIG. 2 is a front view of an example of a section of the frame of the prosthetic heart valve of FIG. 1, as if cut longitudinally and laid flat on a table.

FIG. 3 is a front view of an example of a prosthetic leaflet of the prosthetic heart valve of FIG. 1, as if laid flat on a table.

FIG. 4 is a top view of the prosthetic heart valve of FIG. 1 mounted on an example of a portion of a delivery system.

FIG. 5 is an enlarged view of the handle of the delivery system shown in FIG. 4.

FIG. 6 is an enlarged view of a distal end of the delivery system shown in FIG. 4.

FIG. 7 is a top view of an example of a balloon catheter when the balloon is inflated.

FIG. 8 is a top view of an example of an inflation system for use with a delivery system similar to that shown in FIG. 4.

FIG. 9 is a side view of the inflation system of FIG. 8.

FIG. 10 is a perspective view of a connection between the inflation system of FIGS. 8-9 and the handle of the delivery system of FIG. 4.

FIG. 11 is a flowchart showing exemplary steps in a procedure to implant the prosthetic heart valve of FIG. 1 into a patient using the delivery system of FIG. 4.

FIG. 12A is a side view of a mechanical crimper according to an aspect of the disclosure.

FIG. 12B is a perspective view of the mechanical crimper of FIG. 12A with certain components being shown in phantom.

FIG. 12C is a partially-exploded view of a crimper having components in common with the crimper of FIG. 12A, but having a different housing configuration.

FIG. 12D is a top view of a crimper having components in common with the crimper of FIGS. 12A-B, but having another different housing configuration.

FIG. 13A is a side view of the crimper of FIG. 12A with certain components shown in phantom.

FIG. 13B is a side view of the crimper of FIG. 13A, with certain components removed from the view to better see interior components.

FIG. 13C provides the same view as FIG. 13B, with a first crimping guide shown in addition to the components of FIG. 13B, the first crimping guide shown in phantom.

FIG. 13D provides the same view as FIG. 13B, with a second crimping guide shown in addition to the components of FIG. 13B, the second crimping guide shown in phantom.

FIG. 14 is a top view of the crimper of FIG. 12A showing a representation of a zone of interference.

FIGS. 15A-B are front and perspective views, respectively, of a portion of the crimper of FIG. 12A implanting a positive stop mechanism.

FIGS. 16A-C are various views illustrating a hard stop mechanism for used with a large gear.

FIG. 17 is a perspective view of a mechanical crimper according to an aspect of the disclosure.

FIG. 18 is an enlarged perspective view of a sizing key positioned adjacent the housing assembly of the mechanical crimper of FIG. 17.

FIG. 19 shows the crimper of FIG. 17 with the handle assembly removed and a portion of the housing assembly omitted to better illustrate internal components of the crimper.

FIG. 20 shows an enlarged view of the sizing key coupled to the crimper of FIG. 17.

FIG. 21 shows an enlarged view of certain portions of the internal components shown in FIG. 19 in a state of engagement.

FIG. 22 shows an enlarged view of certain portions of the internal components shown in FIG. 19 in a state of disengagement

FIG. 23 is a flow chart showing example steps of an example method of using the crimper of FIG. 17.

FIG. 24 is a perspective view of a crimper in a closed condition according to another aspect of the disclosure.

FIG. 25 is a perspective view of the crimper of FIG. 24 in an open condition.

FIGS. 26-27 are side and perspective views, respectively, of the crimper of FIGS. 24-25 with portions of the housing and components thereof shown in phantom.

FIG. 28 is a perspective view of the crimper of FIGS. 24-25 with a portion of the housing thereof removed and components of the crimper shown in phantom.

FIGS. 29-30 are enlarged views of a holding mechanism within the housing assembly of the crimper of FIGS. 24-25, the housing assembly being shown in phantom.

FIG. 31 is a perspective view of a retainer according to an aspect of the disclosure.

FIG. 32 is a perspective view of the retainer of FIG. 31 assembled to the crimper of FIGS. 24-25.

FIG. 33 is a cross section of a retainer, according to another aspect of the disclosure, assembled to a catheter, with the retainer in a clamped condition.

FIG. 34 is a side view of the retainer of FIG. 33 in an unclamped condition after disassembly from the catheter.

FIG. 35 is a side view of the retainer of FIG. 33 in a clamped condition after being removed from the crimper.

FIG. 36 is a perspective view of the retainer of FIG. 31 in a clamped condition over the catheter and after assembly to the crimper of FIGS. 24-25.

FIG. 37 is a perspective view of the retainer of FIG. 31 in a clamped condition after disassembly from the catheter and after to being removed from the crimper of FIGS. 24-25.

FIG. 38 is a perspective view of a crimper according to another aspect of the disclosure.

FIG. 39 is a partially-exploded view of the crimper of FIG. 38.

### DETAILED DESCRIPTION OF THE DISCLOSURE

As used herein, the term “inflow end” when used in connection with a prosthetic heart valve refers to the end of the prosthetic valve into which blood first enters when the prosthetic valve is implanted in an intended position and orientation, while the term “outflow end” refers to the end of the prosthetic valve where blood exits when the prosthetic valve is implanted in the intended position and orientation. Thus, for a prosthetic aortic valve, the inflow end is the end nearer the left ventricle while the outflow end is the end nearer the aorta. The intended position and orientation are used for the convenience of describing valves disclosed herein. However, it should be noted that the use of the valve is not limited to the intended position and orientation but may be deployed in any type of lumen or passageway. For example, although prosthetic heart valves are described herein as prosthetic aortic valves, those same or similar structures and features can be employed in other heart valves, such as the pulmonary valve, the mitral valve, or the tricuspid valve. Further, the term “proximal,” when used in connection with a delivery device or system, refers to a position relatively close to the user of that device or system when it is being used as intended, while the term “distal” refers to a position relatively far from the user of the device. In other words, the leading end of a delivery device or system is positioned distal to the trailing end of the delivery device or system, when the delivery device is being used as intended. As used herein, the terms “substantially,” “generally,” “approximately,” and “about” are intended to mean that slight deviations from absolute are included within the scope of the term so modified. As used herein, the prosthetic heart valves may assume an “expanded state” and a “collapsed state,” which refer to the relative radial size of the stent.

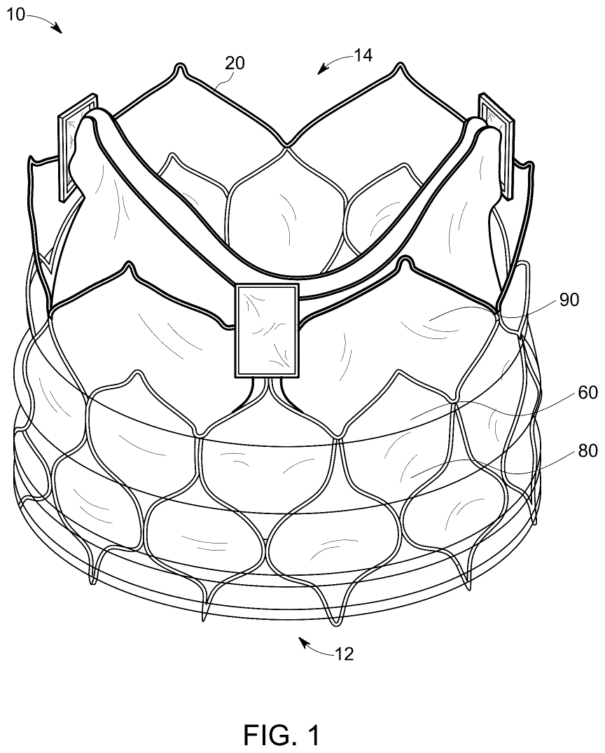

FIG. 1 is a perspective view of one example of a prosthetic heart valve 10. Prosthetic heart valve 10 may be a balloon-expandable prosthetic aortic valve, although in other examples it may be a self-expandable or mechanically-expandable prosthetic heart valve, intended for replacing a native aortic valve or another native heart valve. Prosthetic heart valve 10 is shown in an expanded condition in FIG. 1. Prosthetic heart valve 10 may extend between an inflow end 12 and an outflow end 14. Prosthetic heart valve 10 may include a collapsible and expandable frame 20, an inner cuff or skirt 60, an outer cuff or skirt 80, and a plurality of prosthetic leaflets 90. As should be clear below, prosthetic heart valve 10 is merely one example of a prosthetic heart valve, and other examples of prosthetic heart valves may be suitable for use with the concepts described below.

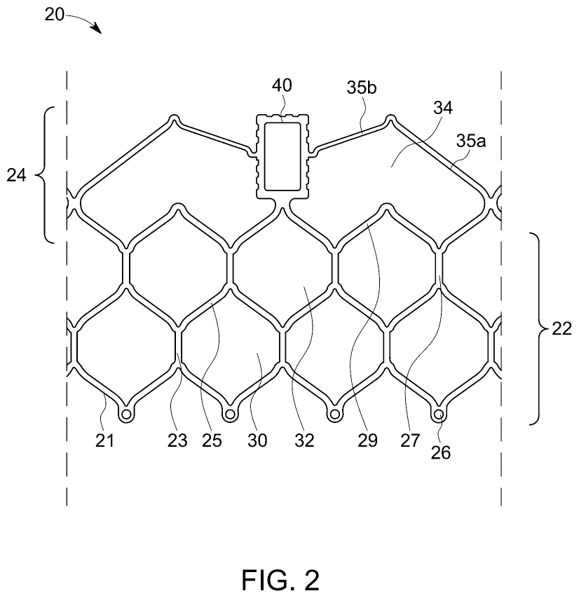

FIG. 2 is a front view of an example of a section of the frame 20 of prosthetic heart valve 10, as if cut longitudinally and laid flat on a table. The section of frame 20 in FIG. 2 may represent approximately one-third of a complete frame, particularly if frame 20 is used in conjunction with a three-leaflet prosthetic heart valve. In the illustrated example, frame 20 is a balloon-expandable stent and may be formed of stainless steel or cobalt-chromium, and which may include additional materials such as nickel and/or molybdenum. However, in some embodiments the stent may be formed of a shape memory material such as nitinol or the like. The frame 20, when provided as a balloon-expandable frame, is configured to collapse upon being crimped to a smaller diameter and/or expand upon being forced open, for example via a balloon within the frame expanding, and the frame will substantially maintain the shape to which it is modified when at rest.

Frame 20 may include an inflow section 22 and an outflow section 24. The inflow section 22 may also be referred to as the annulus section. In one example, the inflow section 22 includes a plurality of rows of generally hexagon-shaped cells. For example, the inflow section 22 may include an inflow-most row of hexagon-shaped cells 30 and an outflow-most row of hexagon-shaped cells 32. The inflow-most row of hexagonal cells 30 may be formed of a first circumferential row of angled or zig-zag struts 21, a second circumferential row of angled or zig-zag struts 25, and a plurality of axial struts 23 that connect the two rows. In other words, each inflow-most hexagonal cell 30 may be formed by two angled struts 21 that form an apex pointing in the inflow direction, two angled struts 25 that form an apex pointing in the outflow direction, and two axial struts that connect the two angled struts 21 to two corresponding angled struts 25. The outflow-most row of hexagonal cells 32 may be formed of the second circumferential row of angled or zig-zag struts 25, a third circumferential row of angled or zig-zag struts 29, and a plurality of axial struts 27 that connect the two rows. In other words, each outflow-most hexagonal cell 32 may be formed by two angled struts 25 that form an apex pointing in the inflow direction, two angled struts 29 that form an apex pointing in the outflow direction, and two axial struts that connect the two angled struts 27 to two corresponding angled struts 29. It should be understood that although the term “outflow-most” is used in connection with hexagonal cells 32, additional frame structure, described in more detail below, is still provided in the outflow direction relative to the outflow-most row of hexagonal cells 32.

In the illustrated embodiment, assuming that frame 20 is for use with a three-leaflet valve and thus the section shown in FIG. 2 represents about one-third of the frame 20, each row of cells 30, 32 includes twelve individual cells. However, it should be understood that more or fewer than twelve cells may be provided per row of cells. Further, the inflow or annulus section 22 may include more or fewer than two rows of cells. Still further, although cells 30, 32 are shown as being hexagonal, the some or all of the cells of the inflow section 22 may have other shapes, such as diamond-shaped, chevron-shaped, or other suitable shapes. In the illustrated embodiment, every cell 30 in the first row is structurally similar or identical to every other cell 30 in the first row, every cell 32 in the second row is structurally similar or identical to every other cell 32 in the second row, and every cell 30 in the first row is structurally similar or identical (excluding the aperture 26) to every cell 32 in the second row. However, in other examples, the cells in each row are not identical to every other cell in the same row or in other rows.

An inflow apex of each hexagonal cell 30 may include an aperture 26 formed therein, which may accept sutures or similar features which may help couple other elements, such as an inner cuff 60, outer cuff 80, and/or prosthetic leaflets 90, to the frame 20. However, in some examples, one or more or all of the apertures 26 may be omitted.

Still referring to FIG. 2, the outflow section 24 of the frame 20 may include larger cells 34 that have generally asymmetric shapes. For example, the lower or inflow part of the larger cells 34 may be defined by the two upper struts 29 of a cell 32, and one upper strut 29 of each of the two adjacent cells 32. In other words, the lower end of each larger cell 34 may be formed by a group of four consecutive upper struts 29 of three circumferentially adjacent cells 32. The tops of the larger cells 34 may each be defined by two linking struts 35a, 35b. The first linking strut 35a may couple to a top or outflow apex of a cell 32 and extend upwards at an angle toward a commissure attachment feature (“CAF”) 40. The second linking strut 35b may extend from an end of the first linking strut 35a back downwardly at an angle and connect directly to the CAF 40. To the extent that the larger cells 34 include sides, a first side is defined by a portion of the CAF 40, and a second side is defined by the connection between first linking strut 35a and the corresponding upper strut 29 of the cell 32 attached to the first linking strut 35a.

The CAF 40 may generally serve as an attachment site for leaflet commissures (e.g. where two prosthetic leaflets 90 join each other) to be coupled to the frame 20. In the illustrated example, the CAF 40 is generally rectangular and has a longer axial length than circumferential width. The CAF 40 may define an interior open rectangular space. The struts that form CAF 40 may be generally smooth on the surface defining the open rectangular space, but some or all of the struts may have one or more suture notches on the opposite surfaces. For example, in the illustrated example, CAF 40 includes two side struts (on the longer side of the rectangle) and one top (or outflow) strut that all include alternating projections and notches on their exterior facing surfaces. These projections and notches may help maintain the position of one or more sutures that wrap around these struts. These sutures may directly couple the prosthetic leaflets 90 to the frame 20, and/or may directly couple an intermediate sheet of material (e.g. fabric or tissue) to the CAF 40, with the prosthetic leaflets 90 being directly coupled to that intermediate sheet of material. In some embodiments, tabs or ends of the prosthetic leaflets 90 may be pulled through the opening of the CAF 40, but in other embodiments the prosthetic leaflets 90 may remain mostly or entirely within the inner diameter of the frame 20. It should be understood that balloon-expandable frames are typically formed of metal or metal alloys that are very stiff, particularly in comparison to self-expanding frames. At least in part because of this stiffness, although the prosthetic leaflets 90 may be sutured or otherwise directly coupled to the frame at the CAFs 40, it may be preferable that most or all of the remaining portions of the prosthetic leaflets 90 are not attached directly to the frame 20, but are rather attached directly to an inner skirt 60, which in turn is directly connected to the frame 20. Further, it should be understood that other shapes and configurations of CAFs 40 may be appropriate. For example, various other suitable configurations of frames and CAFs are described in greater detail in U.S. Provisional Patent Application No. 63/579,378, filed Aug. 29, 2023 and titled “TAVI Deployment Accuracy-Stent Frame Improvements,” the disclosure of which is hereby incorporated by reference herein.

With the example described above, frame 20 includes two rows of hexagon-shaped cells 30, 32, and a single row of larger cells 34. In a three-leaflet embodiment of a prosthetic heart valve that incorporates frame 20, each row of hexagon-shaped cells 30, 32 includes twelve cells, while the row of larger cells includes six larger cells 34. As should be understood, the area defined by each individual cell 30, 32 is significantly smaller than the area defined by each larger cell 34 when the frame 20 is expanded. There is also significantly more structure (e.g. struts) that create each row of individual cells 30, 32 than structure that creates the row of larger cells 34.

One consequence of the above-described configuration is that the inflow section 22 has a higher cell density than the outflow section 24. In other words, the total numbers of cells, as well as the number of cells per row of cells, is greater in the inflow section 22 compared to the outflow section 24. The configuration of frame 20 described above may also result in the inflow section 22 being generally stiffer than the outflow section 24 and/or more radial force being required to expand the inflow section 22 compared to the outflow section 24, despite the fact that the frame 20 may be formed of the same metal or metal alloy throughout. This increased rigidity or stiffness of the inflow section 22 may assist with anchoring the frame 20, for example after balloon expansion, into the native heart valve annulus. The larger cells 34 in the outflow section 24 may assist in providing clearance to the coronary arteries after implantation of the prosthetic heart valve 10. For example, after implantation, one or more coronary ostia may be positioned above the frame 20, for example above the valley where two adjacent larger cells 34 meet (about halfway between a pair of circumferentially adjacent CAFs 40). Otherwise, one or more coronary ostia may be positioned in alignment with part of the large interior area of a larger cell 34 after implantation. Either way, blood flow to the coronary arteries is not obstructed, and a further procedure that utilizes the coronary arteries (e.g. coronary artery stenting) will not be obstructed by material of the frame 20. Still further, the lower rigidity of the frame 20 in the outflow section 24 may cause the outflow section 24 to preferentially foreshorten during expansion, with the inflow section 22 undergoing a relatively smaller amount of axial foreshortening. This may be desirable because, as the prosthetic heart valve 10 expands, the position of the inflow end of the frame 20 may remain substantially constant relative to the native valve annulus, which may make the deployment of the prosthetic heart valve 10 more precise. This may be, for example, because the inflow end of the frame 20 is typically used to gauge proper alignment with the native valve annulus prior to deployment, so axial movement of the inflow end of the frame 20 relative to the native valve annulus during deployment may make precise placement more difficult.

Referring back to FIG. 1, the prosthetic heart valve 10 may include an inner skirt 60 mounted to the interior surface of frame 20. The inner skirt 60 may be formed of tissue, such as pericardium, although other types of tissue may be suitable. In the illustrated example, the inner skirt 60 is formed of a woven synthetic fabric, such as polyethylene terephthalate (“PET”) or polytetrafluoroethylene (“PTFE”), although other fabrics may be suitable, including fabrics other than woven fabrics. In some examples, the inner skirt 60 has straight or zig-zag shaped inflow and outflow ends that generally follow the contours of the cells 30, 32 of the inflow section 22 of frame 20. Preferably, inner skirt 60 is sutured to the frame 20 along the struts that form cells 30, 32. If apertures 26 are included, inner skirt 60 may also be coupled to frame 20 via sutures passing through apertures 26. Preferably, the inner skirt 60 does not cover (or does not cover significant portions of) the larger cells 34. The inner skirt 60 may be coupled to the frame 20 via mechanisms other than sutures, including for example ultrasonic welding or adhesives. Further, the inner skirt 60 may have shapes other than that shown, and need not have a zig-zag inflow or outflow end, and need not cover every cell in the inflow section 22. In fact, in some examples, the inner skirt 60 may be omitted entirely, with the outer skirt 80 (described in greater detail below) being the only skirt used with prosthetic heart valve 10. If the inner skirt 60 is provided, it may assist with sealing the prosthetic heart valve 10 within the heart, as well as serving as a mounting structure for the prosthetic leaflets 90 (described in greater detail below) within the frame 20.

Still referring to FIG. 1, the prosthetic heart valve 10 may include an outer skirt 60 mounted to the exterior surface of frame 20. The outer skirt 80 may be formed of tissue, such as pericardium, although other types of tissue may be suitable. In the illustrated example, the outer skirt 80 is formed of a woven synthetic fabric, such as PET or PTFE, although other fabrics may be suitable, including fabrics other than woven fabrics. In some examples, the outer skirt 80 has straight or zig-zag inflow end. Preferably, outer skirt 80 is sutured to the frame 20 and/or inner skirt 60 along the inflow edge of the outer skirt 80. If apertures 26 are included, outer skirt 80 may also be coupled to frame 20 via sutures passing through apertures 26. The outer skirt 80 may include a plurality of folds or pleats, such a circumferentially extending folds or pleats. The folds or pleats may be formed in the outer skirt 80 via heat setting, for example by placing the outer skirt 80 within a mold that forces the outer skirt 80 to form folds of pleats, and the outer skirt 80 may be treated with heat so that the outer skirt 80 tends to maintain folds or pleats in the absence of applied forces. The outflow edge of outer skirt 80 may be coupled to the frame 20 at selected, spaced apart locations around the circumference of the frame 20. In some embodiments, the outflow edge of outer skirt 80 may be connected to the inner skirt 60 along a substantially continuous suture line. Some or all of the outer skirt 80 between its inflow and outflow edges may remain not directly couples to the frame 20 or inner skirt 60. Preferably, the outer skirt 80 does not cover (or does not cover significant portions of) the larger cells 34. In use, the outer skirt 80 may directly contact the interior surface of the native heart valve annulus to assist with sealing, including sealing against PV leak. If folds or pleats are included with the outer skirt 80, the additional material of the folds or pleats may help further mitigate PV leak. However, it should be understood that the folds or pleats may be omitted from outer skirt 80, and the outer skirt 80 may have shapes other than that shown. In fact, in some examples, the outer skirt 80 may be omitted entirely, with the inner skirt 60 being the only skirt used with prosthetic heart valve 10. If the inner skirt 60 is omitted, the prosthetic leaflets 90 may be attached directly to the frame 20 and/or directly to the outer skirt 80.

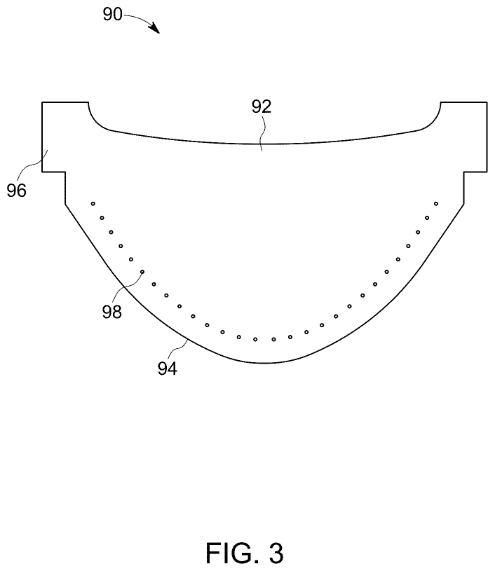

FIG. 3 is a front view of a prosthetic leaflet 90, as if laid flat on a table. In the illustrated example of prosthetic heart valve 10, a total of three prosthetic leaflets 90 are provided, although it should be understood that more or fewer than three prosthetic leaflets may be provided in other example of prosthetic heart valves. The prosthetic leaflet 90 may be formed of a synthetic material, such a polymer sheet or woven fabric, or a biological material, such a bovine or porcine pericardial tissue. However, other materials may be suitable. In on example, the prosthetic leaflet 90 is formed to have a concave free edge 92 configured to coapt with the free edges of the other leaflets to help provide the one-way valve functionality. The prosthetic leaflet 90 may include an attached edge 94 which is attached (e.g. via suturing) to other structures of the prosthetic heart valve 10. For example, the attached edge 94 may be coupled directly to the inner skirt 60, directly to the frame 20, and/or directly to the outer skirt 80. It may be preferable that the attached edge 94 is coupled directly only to the inner skirt 60, which may help reduce stresses on the prosthetic leaflet 90 compared to if the attached edge 94 were coupled directly to the frame 20. In some embodiments, a plurality of holes 98 may be formed along the attached edge 94 (or a spaced distance therefrom), for example via lasers. If included, the holes 98 may be used to receive sutures therethrough, which may make it easier to couple the prosthetic leaflet 90 to the inner skirt 60 during manufacturing. For example, the holes 98 may serve as guides if suturing is performed manually, and if the positions of the holes 98 are controlled via the use of layers, the holes 98 may be consistently placed among different prosthetic leaflets 90 to reduce variability between different prosthetic leaflets 90. Leaflet tabs 96 may be provided at the junctions between the free edge 92 and the attached edge 94. Each leaflet tab 96 may be joined to a leaflet tab of an adjacent prosthetic leaflet to form prosthetic leaflet commissures, which may be coupled to the frame 20 via CAFs 40.

The prosthetic heart valve 10 may be delivered via any suitable transvascular route, for example transapically or transfemorally. Generally, transapical delivery utilizes a relatively stiff catheter that pierces the apex of the left ventricle through the chest of the patient, inflicting a relatively higher degree of trauma compared to transfemoral delivery. In a transfemoral delivery, a delivery device housing or supporting the valve is inserted through the femoral artery and advanced against the flow of blood to the left ventricle. In either method of delivery, the valve may first be collapsed over an expandable balloon while the expandable balloon is deflated. The balloon may be coupled to or disposed within a delivery system, which may transport the valve through the body and heart to reach the aortic valve, with the valve being disposed over the balloon (and, in some circumstances, under an overlying sheath). Upon arrival at or adjacent to the aortic valve, a surgeon or operator of the delivery system may align the prosthetic valve as desired within the native valve annulus while the prosthetic valve is collapsed over the balloon. When the desired alignment is achieved, the overlying sheath, if included, may be withdrawn (or advanced) to uncover the prosthetic valve, and the balloon may then be expanded causing the prosthetic valve to expand in the radial direction, with at least a portion of the prosthetic valve foreshortening in the axial direction.

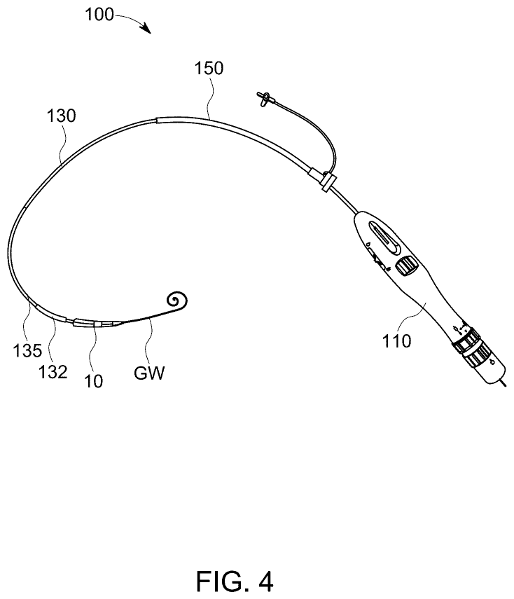

FIG. 4 illustrates one example of a delivery system 100, with the prosthetic heart valve 10 crimped over a balloon on a distal end of the delivery system 100. Although delivery system 100 and various components thereof are described below, it should be understood that delivery system 100 is merely one example of a balloon catheter that may be appropriate for use in delivering and deploying prosthetic heart valve 10.

In some examples, delivery system 100 includes a handle 110 and a delivery catheter 130 extending distally from the handle 110. An introducer 150 may be provided with the delivery system 100. Introducer 150 may be an integrated or captive introducer, although in other embodiments introducer 150 may be a non-integrated or non-captive introducer. In some examples, the introducer 150 may be an expandable introducer, including for example an introducer that expands locally as a large diameter components passes through the introducer, with the introducer returning to a smaller diameter once the large diameter components passes through the introducer. In other examples, the introducer 150 is a non-expandable introducer.

A guidewire GW may be provided that extends through the interior of all components of the delivery system 100, from the proximal end of the handle 110 through the atraumatic distal tip 138 of the delivery catheter 130. The guidewire GW may be introduced into the patient to the desired location, and the delivery system 100 may be introduced over the guidewire GW to help guide the delivery catheter 130 through the patient's vasculature over the guidewire GW.

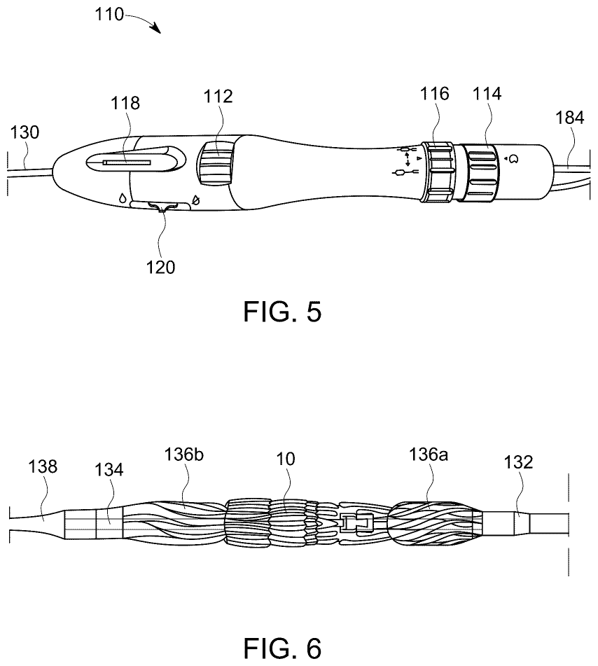

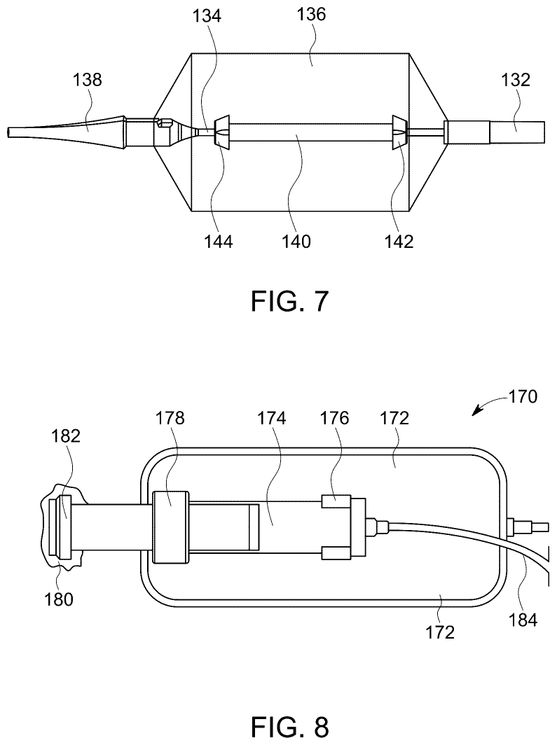

In some examples, the delivery catheter 130 is steerable. For example, one or more steering wires may extend through a wall of the delivery catheter 130, with one end of the steering wire coupled to a steering ring coupled to the delivery catheter 130, and another end of the steering wire operable coupled to a steering actuator on the handle 110. In such examples, as the steering actuator is actuated, the steering wire is tensioned or relaxed to cause deflection or straightening of the delivery catheter 130 to assist with steering the delivery catheter 130 to the desired position within the patient. For example, FIG. 5 is an enlarged view of the handle 110. Handle 110 may include a steering knob 112 that, upon rotation, tensions or relaxes the steering wires to deflect the distal end of the delivery catheter 130. However, it should be understood that the steering functionality may be omitted in some examples, and in other examples steering actuators other than knobs may be utilized. Further, in some examples, including those shown in FIGS. 6-7, the delivery catheter 130 includes an outer catheter 132, and an inner catheter 134. The inner catheter 134 may also be referred to as a guidewire catheter. The steering functionality may be provided in either the outer catheter 132, or the inner catheter 134, or in both catheters. However, in some examples, a separate steering catheter 135 may be provided. For example, as shown in FIG. 4, the steering catheter 135 may be positioned outside of the outer catheter 132 and may terminate just proximal to the balloon 136. With this configuration, deflection of the steering catheter 135 will also cause deflection of the outer catheter 132 and the inner catheter 134 which are both nested within the steering catheter 135. In some examples, the handle may include a window 118 that allows viewing of an indicator that corresponds to the amount of catheter deflection. For example, a carrier to which the indicator is attached may be attached to the steering wire. In some examples, when there is minimum (or zero) tension on the steering wire, the indicator is at the far distal position within window 118, but as deflection is actuated, for example by drawing a carrier proximally (and tensioning the steering wire as the carrier draws proximally), the indicator will move proximally along window 118, giving the user a readily-apparent indication of the amount of deflection applied to the catheter at any given moment.

Still referring to FIGS. 4-5, the delivery system 100 may include additional functionality to assist with positioning the prosthetic heart valve 10. For example, in the illustrated example, handle 110 includes a commissure alignment actuator 114, which may be positioned near a proximal end of the handle or at any other desired location. In the illustrated example, the commissure alignment actuator 114 is in the form of a rotatable knob, although other forms may be suitable. The commissure alignment knob 114 may be rotationally coupled to a portion of the delivery catheter 130 supporting the prosthetic heart valve 10. For example, the commissure alignment actuator 114 may be rotationally coupled to an inner catheter 134 which supports the prosthetic heart valve 10 in the crimped condition. With this configuration, rotating the commissure alignment knob 114 may cause the inner catheter 134 to rotate about its longitudinal axis, and thus cause the prosthetic heart valve 10 to rotate about its longitudinal axis. If a commissure alignment actuator 114 is included, it may be used to help ensure that, upon deployment of the prosthetic heart valve 10 into the native valve annulus, the commissures of the prosthetic heart valve are in rotational alignment with respective ones of the native valve commissures (e.g. within +/−2.5 degrees of rotational alignment, within +/−5 degrees of rotational alignment, within +/−10 degrees of rotational alignment, within +/−15 degrees of rotational alignment, etc.). Although commissure alignment actuator 114 is shown in this example as a knob positioned at or near a proximal end of the handle 110, it should be understood that the actuator 114 may take forms other than a knob, may be positioned at other suitable locations, and may be omitted entirely if desired.

Still referring to FIGS. 4-5, the delivery system 100 may include even further functionality to assist with positioning the prosthetic heart valve 10. For example, in the illustrated example, handle 110 includes an axial alignment actuator 116, which may be positioned near a proximal end of the handle, including distal to the commissure alignment actuator 114, or at any other desired location. In the illustrated example, the axial alignment actuator 116 is in the form of a rotatable knob, although other forms may be suitable. The axial alignment knob 116 may be operably coupled to a portion of the delivery catheter 130 supporting the prosthetic heart valve 10. For example, the axial alignment actuator 116 may include internal threads that engage external threads (or another component, such as individual extensions, which may be cylindrical extensions that fit between internal threads of the actuator) of a carriage that is coupled to an inner catheter 134 which supports the prosthetic heart valve 10 in the crimped condition. In such an example, the carriage may be rotatably fixed to the handle 110. With this configuration, rotating the axial alignment knob 116 may cause the carriage to advance distally or retract proximally as the inner threads of the axial alignment knob 116 mesh with the external threads of the carriage, but the carriage is prevented from rotating. As the carriage advances distally or retracts proximally, the inner catheter 134 may correspondingly advance distally or retract proximally, and thus cause the prosthetic heart valve 10 to advanced distally or retract proximally. It should be understood that, if axial alignment actuator 116 is included, it may have a small total range of motion, including for example between about 2 mm and about 15 mm of range of motion, including about 7.5 mm range of motion. In other words, the rough or coarse axial alignment between the prosthetic heart valve 10 and native valve annulus may be achieved by physically advancing the entire delivery catheter 130 by pushing it through the vasculature while holding the handle 110. However, for fine and more controlled adjustment of the axial position of the prosthetic heart valve 10 relative to the native valve annulus, which may be performed just prior to or during deployment of the prosthetic heart valve 10, the axial alignment knob 116 may be used. If an axial alignment actuator 116 is included, it may be used to help ensure that, upon deployment of the prosthetic heart valve 10 into the native valve annulus, the inflow end of the of the prosthetic heart valve is in axial alignment with the inflow aspect of the native valve annulus (e.g. within +/−0.5 mm of axial alignment, within +/−1.0 mm of axial alignment, within +/−1.5 mm of axial alignment, within +/−2.0 mm of axial alignment, etc.). Although axial alignment actuator 116 is shown in this example as a knob positioned at or near a proximal end of the handle 110, it should be understood that the actuator 116 may take forms other than a knob, may be positioned at other suitable locations, and may be omitted entirely if desired.

In addition to steering and positioning actuators, delivery system 100 may include a balloon actuator 120. In the illustrated example, balloon actuator 120 is positioned on the handle 110 near a distal end thereof, and is provided in the form of a switch. Balloon actuator 120 may be actuated to cause inflation or deflation of a balloon 136 that is part of the delivery system 100. For example, referring briefly to FIGS. 6-7, the delivery system 100 may include a balloon 136 that overlies a distal end of inner catheter 134 and which receives the prosthetic heart valve 10 in a crimped condition thereon. In the example illustrated in FIG. 6, the balloon 136 includes a proximal pillowed portion 136a, a distal pillowed portion 136b, and a central portion over which the prosthetic heart valve 10 is crimped. The proximal pillow 136a and the distal pillow 136b may form shoulders on each side of the prosthetic heart valve 10, which may help ensure the prosthetic heart valve 10 does not move axially relative to the balloon 136 and/or inner catheter 134 during delivery. The shoulder formed by the distal pillow 136 may also help protect the inflow edge of the prosthetic heart valve 10 from contact with the anatomy during delivery. For example, during a transfemoral delivery, as the distal end of the delivery catheter 130 traverse the sharp bends of the aortic arch (or during initial introduction into the patient), there is a relatively high likelihood the inflow end of the prosthetic heart valve 10 (which is the leading edge during transfemoral delivery) will contact a vessel wall (or a components of an introduction system) causing dislodgment of the prosthetic heart valve 10 relative to the balloon 136. The distal pillow 136 may tend to have an equal or larger outer diameter than the inflow end of the prosthetic heart valve 10 (when the prosthetic heart valve 10 is crimped and the balloon 136 is deflated), which may help ensure the inflow edge of the prosthetic heart valve 10 does not inadvertently contact another structure during delivery. In some examples, the pillowed portions 136a, 136b may be formed via heat setting. Additional related features for use in similar balloon catheter delivery systems are described in greater detail in U.S. Provisional Patent Application No. 63/382,812, filed Nov. 8, 2022 and titled “Prosthetic Heart Valve Delivery and Trackability,” the disclosure of which is hereby incorporated by reference herein.

In order to deploy the prosthetic heart valve 10, the balloon 136 is inflated, for example by actuating the balloon actuator 120 to force fluid (such as saline, although other fluids, including liquids or gases, could be used) into the balloon 136 to cause it to expand, causing the prosthetic heart valve 10 to expand in the process. For example, the balloon actuator 120 may be pressed forward or distally to cause fluid to travel through an inflation lumen within delivery catheter 130 to inflate the balloon 136. In some embodiments, the balloon actuator 120 may take the form of a “momentary switch” in which pushing the balloon actuator 120 forward engages inflation, pulling the balloon actuator 120 proximally engages deflation, and releasing the balloon actuator 120 pauses inflation. This particular example of functionality may allow the physician to precisely control the amount of fluid dispensed while reducing the occurrence of over- or under-inflation, for example because the system automatically pauses inflation when the switch is released. The physical form factor of the balloon actuator 120 may be any suitable desired form factor, including for example a rocker switch, a push button, etc. In some embodiments a second balloon actuator or button may be provided, cither on the balloon actuator 120 or elsewhere on the handle 110, with the second balloon actuator allowing for a change (e.g. increase or decrease) in the rate of inflation, for example to a pre-programmed faster or slower rate of inflation. FIG. 7 illustrates an example of the balloon 136 after being inflated, with the prosthetic heart valve 10 omitted from the figure for clarity. In the illustrated example, the balloon 136 may be formed to have a distal end that is fixed to a portion of an atraumatic distal tip 138. The distal tip 138 may be tapered to help the delivery catheter 130 move through the patient's vasculature more smoothly. A proximal end of the balloon 136 may be fixed to a distal end of outer catheter 132. The inflation lumen may be the space between the outer catheter 132 and the inner catheter 134, or in other embodiments may be provided in a wall of the inner catheter 134, or in any other location that fluidly connects the interior of the balloon 136 to a fluid source outside of the patient that is operable coupled to the delivery system 100.

Referring to FIG. 7, in some examples, a mounting shaft 140 may be provided on the inner catheter 134. A proximal stop 142 and/or a distal stop 144 may be provided, for example at opposite ends of the mounting shaft 140. If the mounting shaft 140 is included, it may provide a location on which the prosthetic heart valve 10 may be crimped. If the proximal stop 142 and/or distal stop 144 is provided, they may provide physical barriers to the prosthetic heart valve 10 moving axially relative to the balloon 136. In one example, the proximal stop 142 may taper from a larger distal diameter to a smaller proximal diameter, and the distal stop may taper from a larger proximal diameter to a smaller distal diameter. The spacing between the proximal stop 142 and the distal stop 144, if both are included, may be slightly larger than the length of the prosthetic heart valve 10 when it is crimped over mounting shaft 140. However, it should be understood that one or both of the stops 142, 144 may be omitted, and the mounting shaft 140 may also be omitted. If the mounting shaft 140 is included, it is preferably axially and rotationally fixed to the inner catheter 134 so that movement of the inner catheter 134 causes corresponding movement of the mounting member 140, and thus the prosthetic heart valve 10 when mounted thereon.

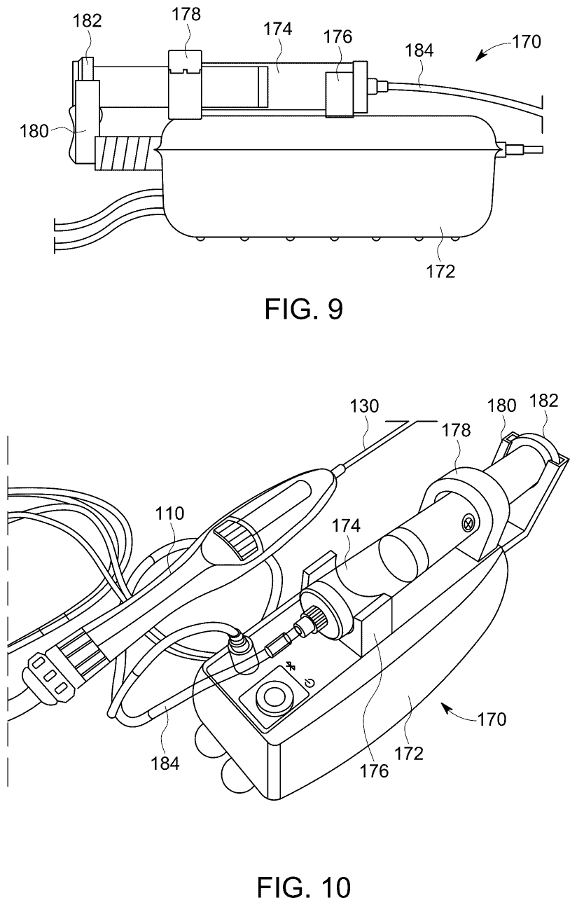

Before describing the use of balloon actuator 120 in more detail, it should be understood that in some embodiments, the balloon actuator 120 may be omitted and instead a manual device, such as a manual syringe, may be provided along with delivery system 100 in order to manually push fluid into balloon 136 during deployment of the prosthetic heart valve 10. However, in the illustrated example of delivery system 100, the balloon actuator 120 provides for a motorized and/or automated (or semi-automated) balloon inflation functionality. For example, FIG. 8 and FIG. 9 illustrate an example of a balloon inflation system 170. Balloon inflation system 170 may include a housing 172 that houses one or more components, which may include a motor, one or more batteries, electronics for control and/or communication with other components, etc. Housing 172 may include one or more fixed cradles to receive a syringe 174. In the illustrated embodiment, a distal cradle 176 is provide with an open “C”- or “U”-shaped configuration so that the distal end of the syringe 174 may be snapped into or out of the distal cradle 176. A proximal cradle 178 may also be provided, which may have a “C”- or “U”-shaped bottom portion hingedly connected to a “C”- or “U”-shaped top portion. This configuration may allow for the proximal end of the outer body of the syringe 174 to be snapped into the bottom portion of proximal cradle 178, and the top portion of proximal cradle 178 may be closed and connected to the bottom portion to fully circumscribe the outer body of the syringe 174 to lock the syringe 174 to the housing 172. It should be understood that more or fewer cradles, of similar or different designs, may be included with housing 172 to help secure the syringe 174 to the housing 172 in any suitable fashion.

The balloon inflation system 170 may include a moving member 180. In the illustrated embodiment, moving member 180 includes a “C”- or “U”-shaped cradle to receive a plunger handle 182 of the syringe 174 therein, the cradle being attached to a carriage that extends at least partially into the housing 172. The carriage of the moving member 180 may be generally cylindrical, and may include internal threading that mates with external threading of a screw mechanism (not shown) within the housing 172 that is operably coupled to a motor. In some embodiments, the carriage may have the general shape of a “U”-beam with the flat face oriented toward the top. The moving member 180 may be rotationally fixed to the housing 172 via any desirable mechanism, so that upon rotation of the screw mechanism by the motor, the moving member 180 advances farther into the housing 172, or retracts farther away from the housing 172, depending on the direction of rotation of the screw mechanism. While the plunger handle 182 is coupled to the moving member 180, advancement of the moving member 180 forces fluid from the syringe 174 toward the balloon 136, while retraction of the moving member 180 withdraws fluid from the balloon 136 toward the syringe 174. It should be understood that the motor, or other driving mechanism, may be located in or outside the housing 172, and any other suitable mechanism may be used to operably couple the motor or other driving mechanism to the moving member 180 to allow for axial driving of the plunger handle 182.

As shown in each of FIG. 8, FIG. 9, and FIG. 10, the distal end of syringe 174 may be coupled to tubing 184 that is in fluid communication with an inflation lumen of delivery catheter 130 that leads to the balloon 136 at or near the distal end of the delivery system 100. Tubing 184 may allow for the passage of the fluid (e.g., saline) from the syringe 174 toward the balloon 136, or for withdrawal of fluid from the balloon 136 toward the syringe 174, for example based on whether the balloon actuator 120 is pressed forward or backward.

Although not separately numbered in FIG. 8, FIG. 9, and FIG. 10, the housing 172 may include one or more cables extending from the housing, for example to allow for transmission of power (e.g. from AC mains or another component with which the cable is coupled) and/or transmission of data, information, control commands, etc. For example, one cable may couple the housing 172 to handle 110 so that controls on the handle 110 (e.g. balloon actuator 120) may be used to activate the balloon inflation system 170 in the desired fashion. Another cable may couple to a computer display or similar device to provide information regarding the inflation of the balloon 136. However, it should be understood that any transmission of data or information may be provided wirelessly instead of via a wired connection, for example via a Bluetooth or other suitable connection. Additional and related features of balloon inflation system 170, related systems, and the uses thereof are described in U.S. patent application Ser. No. 18/311,458, the disclosure of which is hereby incorporated by reference herein.

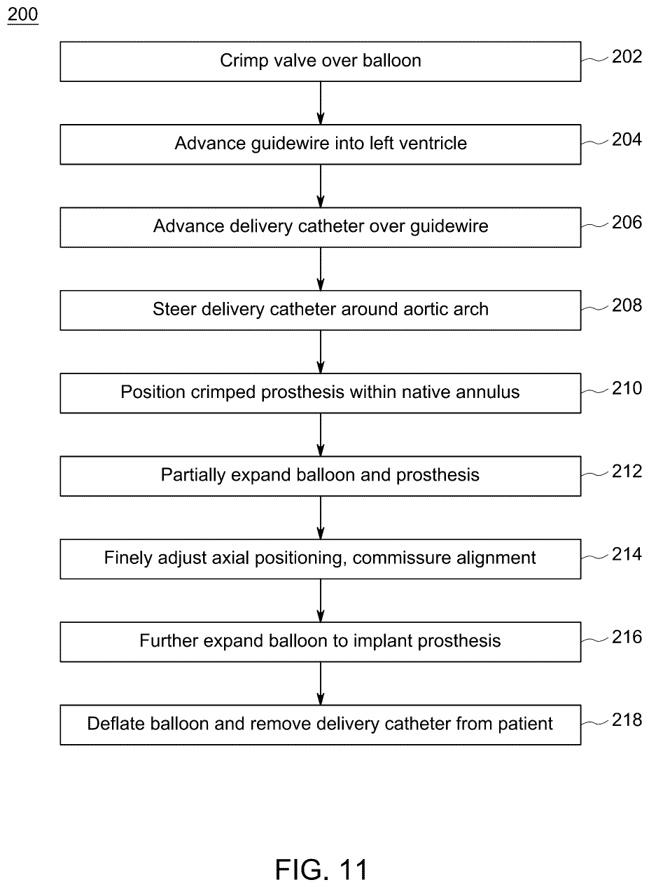

FIG. 11 is a flowchart showing exemplary steps in an implantation procedure 200 to implant the prosthetic heart valve 10 of FIG. 1 into a patient using the delivery system 100 of FIG. 4. However, it should be understood that not all of the steps shown in connection with implantation procedure 200 need to be performed, and various steps not explicitly shown and described in connection with procedure 200 may be performed as part of the implantation procedure. At the beginning of the procedure 200 in step 202, the prosthetic heart valve 10 may be collapsed over or crimped onto balloon 136, with the balloon 136 being mostly or entirely deflated after the crimping procedure. It should be understood that crimping step 202 may be performed at any time prior to the procedure, including at the beginning of the procedure, or at an earlier stage before the delivery system 100 is provided to the end user. In other words, the crimping step 202 may be performed during a manufacturing stage of the delivery system 100 and/or prosthetic heart valve 10. During an early stage of the implantation procedure 200, a guidewire GW may be advanced into the patient in step 204, for example via the femoral artery, around the aortic arch, through the native aortic valve, and into the left ventricle. The guidewire GW may be used as a rail for other devices that need to access this pathway. For example, in step 206, the atraumatic distal tip 138 may be advanced over the proximal end of the guidewire GW, and the delivery catheter 130 may be advanced over guidewire GW toward the native aortic valve. During this initial advancement of the delivery catheter 130 into the patient, the introducer 150 (if included) may be positioned distally, for example so that it covers the prosthetic heart valve 10 or so that it is positioned just proximal to the prosthetic heart valve 10. Advancement of the delivery catheter 130 and introducer 150 may continue until a proximal hub of the introducer is in contact with the patient's skin (or in contact with another device that enters the patient's femoral artery. At this point, the introducer 150 may stop moving axially relative to the patient, with the delivery catheter 130 continuing to advance relative to the introducer 150. If steering capability is provided, the delivery catheter 130 may be steered or deflected at any point to assist with achieving the desired pathway of the delivery catheter 130. As on example, in step 208, the steering knob 112 may be actuated to deflect the distal end of the delivery catheter 130 as it traverses the sharp bends of the aortic arch. Advancement of the delivery catheter 130 may continue in step 210 until the prosthetic heart valve 10, while still crimped or collapsed, is positioned within the native aortic valve annulus. With the desired position achieved, the balloon 136 may be partially inflated, for example by pressing balloon actuator 120 forward, to partially expand the prosthetic heart valve 10 in step 212. In some examples, it is desirable to expand the prosthetic heart valve 10 only partially in step 212, because the position of the prosthetic heart valve 10 (including rotational and/or axial positioning) relative to the native aortic valve annulus may shift during this partial expansion. After the partial expansion of step 212, the user may examine the positioning of the prosthetic heart valve 10 relative to the native aortic valve annulus. If desired, in step 214, the axial positioning of the partially-expanded prosthetic heart valve 10 relative to the native aortic valve annulus may be finely adjusted (e.g. by actuating axial alignment actuator 116) and/or the rotational orientation of the prosthetic heart valve 10 relative to the native aortic valve may be finely adjust (e.g. by actuating commissure alignment actuator 114). When the desired axial alignment is achieved and the desired rotational alignment (e.g. rotational alignment between the prosthetic commissure and the native commissures) is achieved, the balloon 136 may be fully expanded in step 216 to fully expand the prosthetic heart valve 10 and to anchor the prosthetic heart valve 10 in the native aortic valve annulus in the desired position and orientation. After deployment is complete, the balloon 136 may be deflated in step 218, for example by pressing actuating balloon 120 backward, and the delivery catheter 130 and guidewire GW may be removed from the patient to complete the procedure. It should be understood that the nine steps shown in FIG. 11 as part of procedure 200 are merely exemplary of a single example of an implantation procedure, and steps shown may be omitted, steps not shown may be included, and steps may be provided in any order deemed appropriate by the physician and/or medical personnel.

Although various components of a prosthetic heart valve 10 and delivery system 100 are described above, it should be understood that these components are merely intended to provide better context to the systems, features, and/or methods described below. Thus, various components of the systems described above may be modified or omitted as appropriate without affecting the systems, features, and/or methods described below. For example, prosthetic heart valves other than the specific configuration shown and described in connection with FIGS. 1-3 may be used with delivery systems other than the specific configuration shown and described in connection with FIGS. 4-10 as part of an implantation procedure that uses steps other than the specific configuration shown and described in connection with FIG. 11, without affecting the inventive systems, features, and/or methods described below.

As noted above, balloon expandable prosthetic heart valves, including prosthetic heart valve 10, are typically forcibly collapsed or crimped onto a delivery device just prior to being implanted into a patient, for example onto balloon 136 of delivery system 100, or onto another component of the delivery system. And while this disclosure focuses on crimping devices that are useful for forcibly collapsing balloon expandable prosthetic heart valves, it should be understood that the crimping devices described herein may be suitable for crimping or forcibly collapsing other types of devices that incorporate plastically expandable frames, including stents and related stent-like devices.

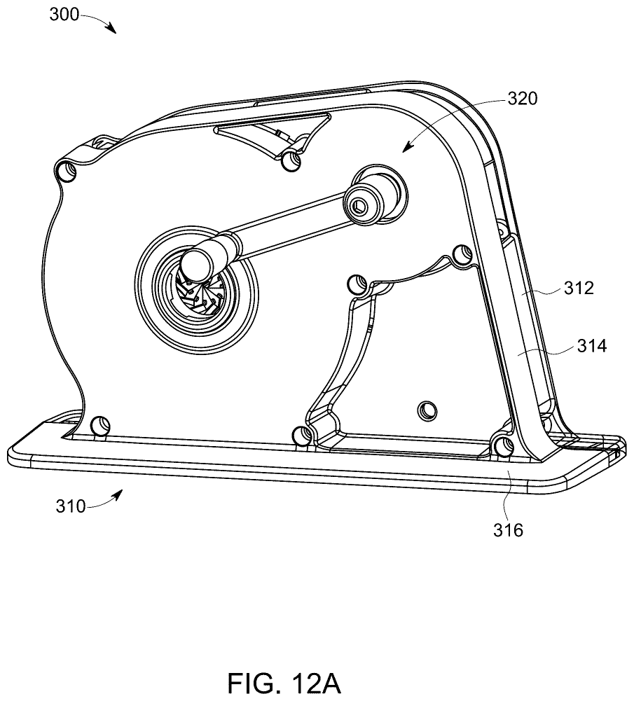

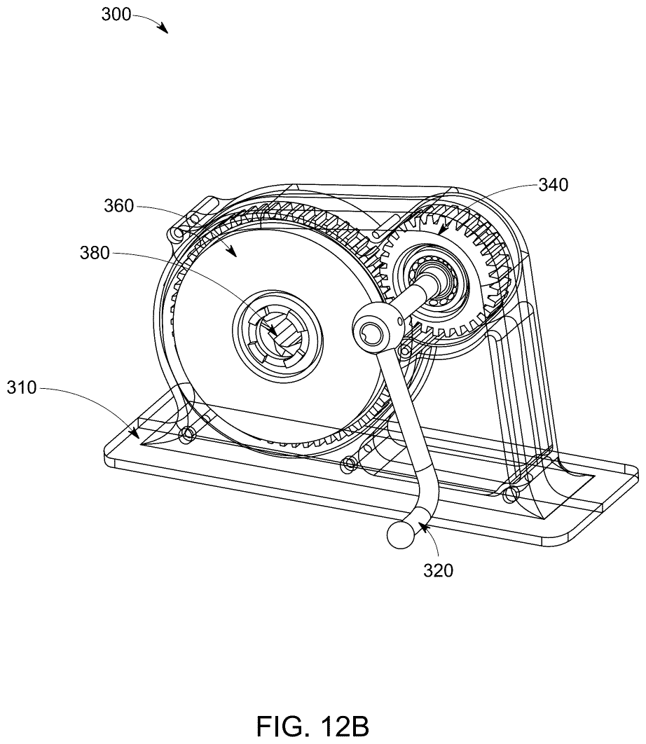

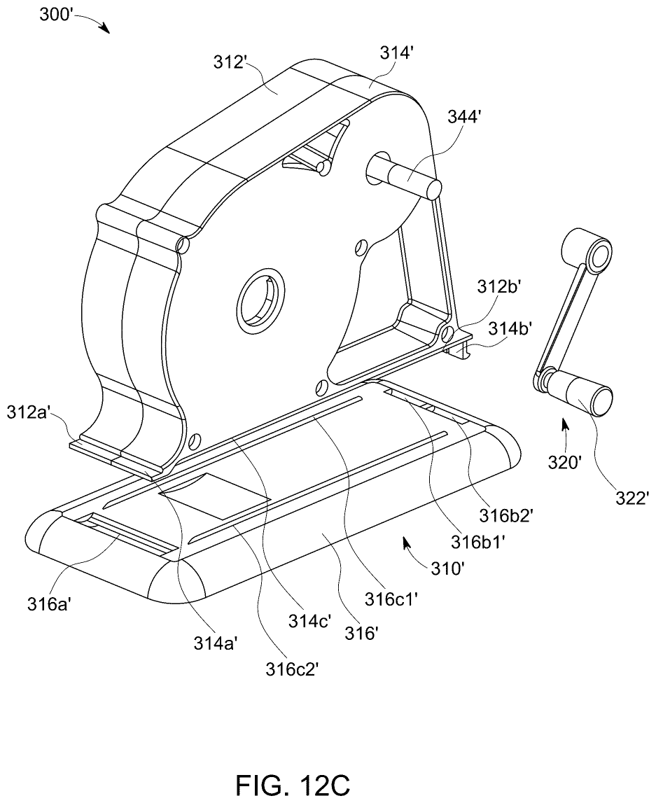

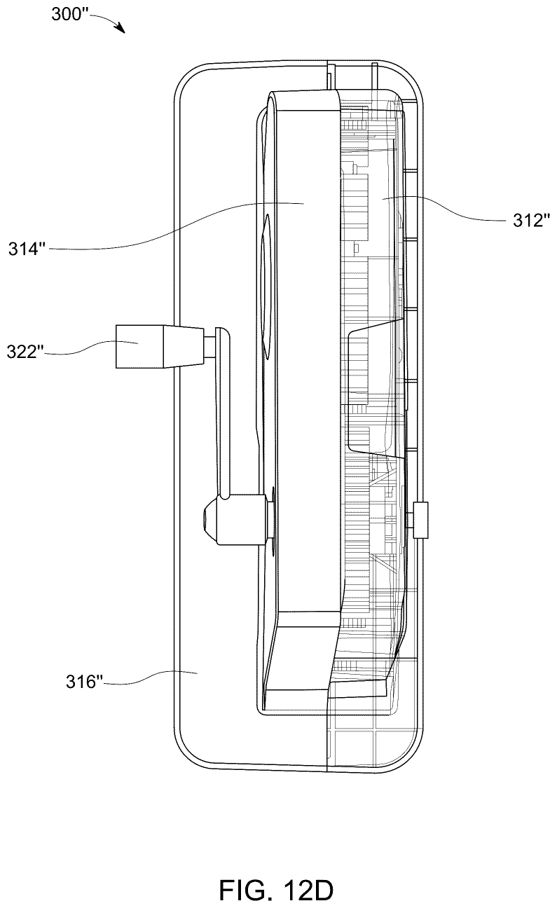

One example of a crimping device 300 (also referred to as a “crimper”) is shown in FIG. 12A. FIG. 12B shows the crimping device 300 with a casement or housing assembly 310 thereof in partial phantom. As shown in FIGS. 12A-12B, the crimping device 300 may generally include a housing assembly 310, an actuator or handle assembly 320, a pinion or first gear assembly 340, a large or second gear assembly 360, and an iris assembly 380. It should be understood that, although the word “assembly” is used above, an “assembly” in some embodiments may include either a single component or multiple components that are structurally or functionally related. On a general level, the crimper 300 may be used by placing a device (such as a prosthetic heart valve, which may be positioned on a balloon or another component of a delivery device, or such as a stent) into the iris assembly 380 while the device is in a relatively expanded condition and while the iris assembly 380 is in a relatively opened condition. Then, the user may rotate the handle assembly 320 to cause the pinion assembly 340 to rotate, which in turn causes the large gear assembly 360 to rotate, which in turn forces the iris assembly 380 to transition to a relatively closed state, forcibly collapsing the device therein into a relatively collapsed condition. While crimpers are generally known in the art, crimper 300 (and variations thereof) may provide one or more benefits over previously known crimpers, including for example an improved layout which may provide one or more advantages to the user and/or mechanical advantage to make the actual crimping process easier for the user.

Referring generally to FIGS. 12A-12B, the housing assembly 310 may include a rear shell 312 and a front shell 314 which may, when assembled, encapsulate or cover most of the components of the crimper 300, with certain exceptions, including for example portions of the handle assembly 320 and the iris assembly 380. The rear shell 312 and front shell 314, when assembled, may form a relatively small compartment to house the pinion assembly 340 and a relatively large compartment to house the large gear assembly 360, with the two compartments connected to each other to allow for contact between the pinion assembly 340 and the large gear assembly 360. The housing assembly 310 may also include a base 316, which may include a generally flat inferior surface to allow for the crimper 300 to stably sit on a table during use. In some examples, base 316 may be provided to the end user already assembled to the rear shell 312 and front shell 314. In these examples, the base 316 may be coupled to the rear shell 312 and front shell 314 in any suitable fashion, including for example snap fit connections, adhesives, welding, or any other suitable connection, including permanent connections. Further, in these examples, the handle assembly 320 may be (but need not be) fully assembled when the crimper 300 is provided to the user.

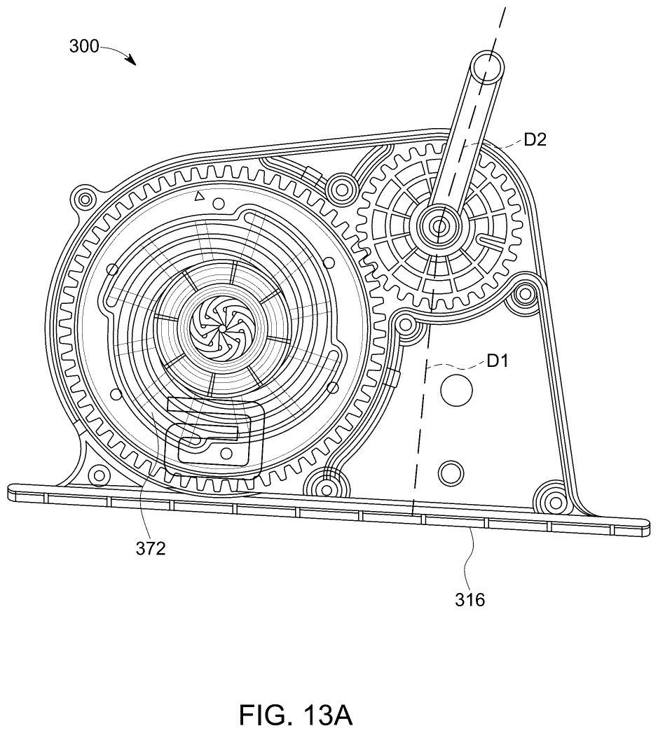

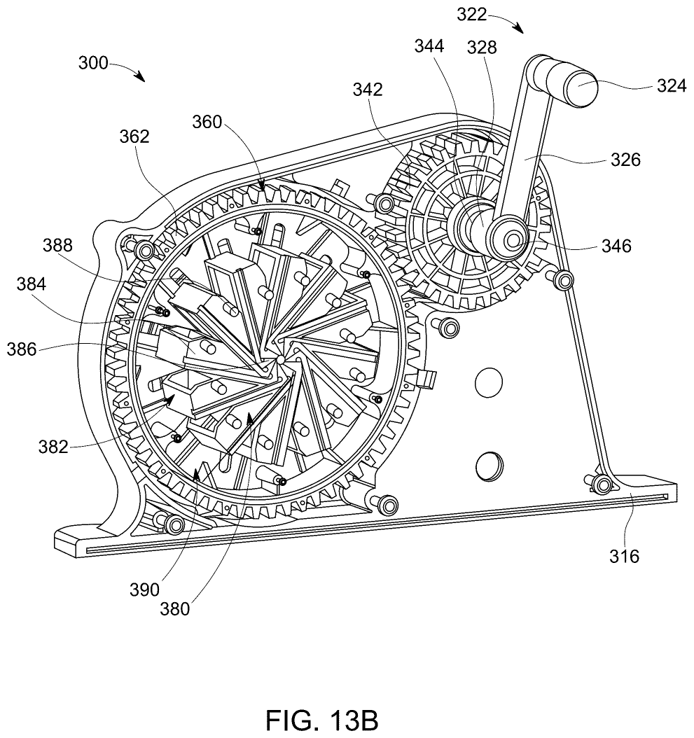

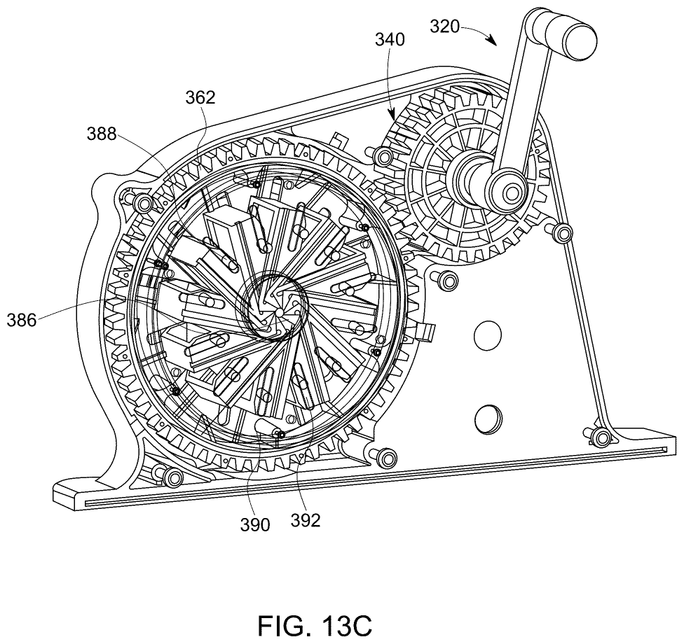

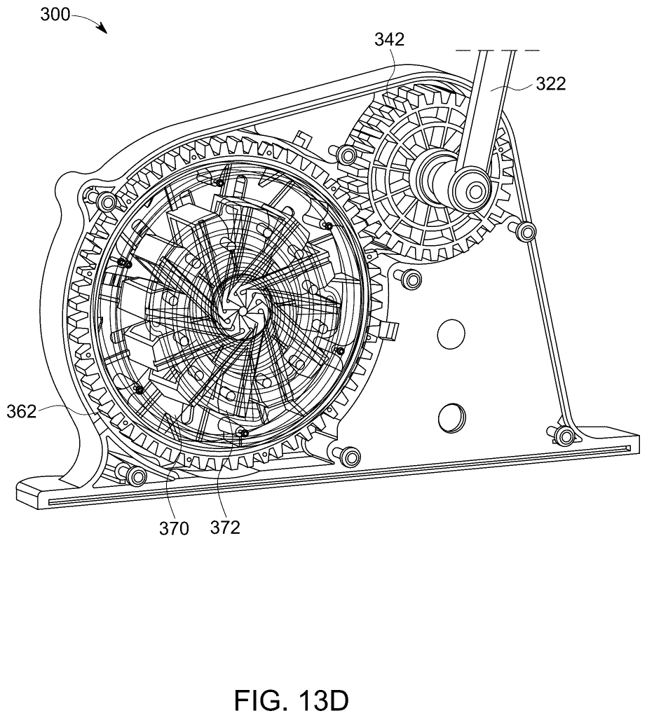

Referring briefly to FIG. 12C, in some embodiments, the crimper 300′ may include a housing assembly 310′ that has a modular design, in which at least some components of the crimper 300′ are intended for assembly by a user (or someone associated with the user) of the crimper 300′. For example, in the example of FIG. 12C, the crimper 300′ includes a housing assembly 310′ that includes a rear shell 312′, front shell 314′ and base 316′ which are each substantially similar in function and design as their counterparts of housing assembly 310, with certain exemplary differences described below. For example, the general design and purpose of rear shell 312′ and front shell 314′ may be nearly identical to the counterpart components 312, 314, including for example that the crimper 300′ may be provided to the end user with the rear shell 312′ and front shell 314′ already assembled. However, the rear shell 312′ and front shell 314′ may each include features configured to snap or otherwise lock into engagement with base 316′. In one example, the rear shell 312′ and front shell 314′ may each include a first tab 312a′, 314a′ respectively that extend generally parallel to the top surface of the base 316′. A horizontal recess 316a′ may be positioned in the top surface of base 316′. During assembly, the first tabs 312a′, 314a′ may first be positioned into the horizontal recess 316a′ while the other end of the base 316′ is angled downwardly away from the remainder of housing assembly 310′. The rear shell 312′ and front shell 314′ may each include a second tab 312b′, 314b′ respectively that extend generally orthogonal (e.g., downward) from the shells 312′, 314′, with a ridge extending rearwardly from each tab 312b′, 314b′. The base 316′ may include two corresponding horizontal recesses 316b1′, 316b2′ in the top surface of the base 316′ on the opposite end of the horizontal recess 316a′. However, it should be understood that the two recesses 316b1′, 316b2′ could be combined into a single continuous recess. Either way, after the tabs 312a′, 314a′ have been inserted into horizontal recess 316a,′ the base 316′ may be angled upwardly until the second tabs 312b′, 314b′ begin to be received within the corresponding recesses 316b1′, 316b2′. The components may be configured such that the second tabs 312b′, 314b′ momentarily flex toward the center of the base 316′, until the ridges are positioned within the recesses 316b1′, 316b2′, at which point the second tabs 312b′, 314b′ may “snap” back with the ridges preventing separation of the base 316′ from the rear shell 312′ and front shell 314′. In some examples, the side edges of each shell may include an axial rail, with only the axial rail 314c′ of front shell 314′ being visible in the view of FIG. 12C. The top surface of base 316′ may include two corresponding axial recesses 316cl′, 316c2′ to accept the complementary rail of the rear shell 312′ and rail 314c′ of the front shell 314′ to help further stabilize the components of the housing assembly 310′ once assembled. In some examples, depending on clearances available, the top surface of the base 316′ may include a divot or recess (best shown in FIG. 12C) that receives a portion of the large gear 362 to ensure the large gear 362 does not contact the top surface of the base 316′ (an example of this is shown in FIGS. 13C-13D).

In some examples, whether the housing assembly is provided as a singular pre-assembled housing assembly 310 or a modular housing assembly 310′, the handle assembly 320′ may be provided with a detachable handle 322′ that may be coupled and removed from a pinion drive shaft 344′, which is described in greater detail below.

It should be understood that, for any of the embodiments and/or features of the crimpers described herein, the crimper may be used with any style of housing assembly and/or handle assembly described herein, unless explicitly stated otherwise.

The housing assemblies 310 and 310′ described above are generally shown with a front shell and a rear shell that are coupled to a base such that the assembled shells are mostly centered between the long side edges of the base. However, either housing assembly 310 or 310′ may be modified to offset the rear and front shells closer to one long side edge of the base and farther from the other long side edge of the base. For example, FIG. 12D shows a top view of a crimper 300″ that may be similar or identical to crimper 300 and/or crimper 300′ in all ways other than the positioning of the rear shell 312″ and front shell 314″ relative to base 316″. As shown in FIG. 12D, the rear shell 312″ and front shell 314″ may be positioned relative to the base 316″ such that both shells are offset toward one of the long side edges of the base 316″ so that the handle 322″, particularly where it connects to the pinion assembly, is generally centered between the long side edges of the base 316″. As should become clear from the description below, when crimping a device using any of the crimpers described herein, a user manually rotates a handle (e.g., handle 322″) to rotate the pinion and large gears to close the iris assembly. This rotation may require significant force that could tend to destabilize the base which sits on a table or another flat surface. One way to prevent such destabilization would be to provide a very wide base, but this may increase the total size and bulk of the crimper, including the size of required packaging, which may be undesirable. Rather, additional stability may be obtained, without changing the size of the base, by positioning the handle 322″ nearer the center of the base 315″ in the lateral or long side-to-long side direction. In other words, when force is applied to the handle 322″, particularly on the downward stroke in the direction toward the base 316″, the force will tend to be centered or nearly centered on the base 316″ (in the lateral direction) which will help prevent the crimper 300″ from tipping over, compared to embodiments in which the force from the downward stroke is positioned close to one of the long sides of the base 316″.

It should be understood that, while multiple particular embodiments of housing assemblies are described above, the housing assemblies could be provided in other configurations while maintaining the some or all of the crimping functionality described below. Therefore, no particular design of the housing assembly is required to achieve at least some of the benefits of the crimper embodiments described herein.

FIG. 13A shows a side view of crimper 300 with the front shell 314 shown in phantom to help illustrate certain internal components of the crimper 300. The description of crimper 300, including the below description in connection with FIGS. 13A-D, may apply to any of the housing embodiments (e.g., singular, modular, and/or with a stabilizing offset) described above, as well as particular housings or casings not explicitly described herein.

FIG. 13B shows a similar side view as FIG. 13A, but in FIG. 13B, the front casing 314 has been removed, and the base 316 has been sectioned. As shown in FIG. 13B, the pinion gear assembly 340 may include a pinion 342 which may take the form of a spur gear. The pinion 342 may have a plurality of pinion teeth extending radially outwardly from an outer circumference thereof, and may define an interior opening along a central axis of the pinion 342. A pinion drive shaft 344 may pass into the interior opening, and the pinion drive shaft 344 and interior opening of the pinion 342 may be geometrically (or otherwise) keyed so that rotation of the pinion drive shaft 344 causes rotation of the pinion 342. In other words, the pinion drive shaft 344 is rotatably fixed to the pinion 342, while the pinion 342 is suspended within the housing assembly 310 so that, when the pinion 342 rotates, it does not contact any surfaces of the housing assembly 310 that would hinder rotation of the pinion 342.

Although not shown in FIG. 13B (see instead, e.g., FIG. 12A), the pinion drive shaft 344 may extend outwardly form the front casing 314 when the housing assembly 310 is fully assembled. The handle assembly 320 may include a handle 322, which may include a first handle end 324 configured to be gripped by a user to actuate pinion gear 342. The handle 322 may include a main handle body 326 extending from the first handle end 324 to a second handle end 328. The second handle end 328 may be hollow so that the exposed portion of the pinion drive shaft 344 is received within the second handle end 328. In some example, a fastener 346 may be provide to fasten the handle 322 to the pinion drive shaft 344, for example due to the fastener 346 having a head with a larger dimension than the interior dimension of the second handle end 328. The second handle end 328 is rotationally fixed to the pinion drive shaft 344, for example via geometric keying or any other suitable fashion, such that rotation of the handle 322 transmits torque to the pinion drive shaft 344, which in turn transmits torque to the pinion gear 342, such that one rotation of the handle 322 creates one rotation of the pinion 342. Although one particular example of handle assembly 320 is described herein, it should be understood that other specific configurations of handles may be implemented in order to allow transmission of torque between handle 322 and pinion 342. In at least some examples, the center axis of the pinion 342 is positioned above (i.e. upward when the base 316 of the crimper 300 sits on a table) the central axis of the iris assembly 380. The purpose of this offset, which may be generally to move most or all of the components of the handle 322 outside the viewing window of the interior of the iris assembly 380, is described in greater detail below.

Still referring to FIG. 13B, the large gear assembly 360 may include a large gear 362 which may take the form of a spur gear. The large gear 362 may have a plurality of gear teeth extending radially outwardly from an outer circumference thereof, and may have a largely hollow interior that houses other components of the large gear assembly 360 and/or the iris assembly 380. Not shown in FIG. 13B, although shown in other figures and described in greater detail below, the large gear assembly 360 may include a cam track 370 which is rotationally fixed to the large gear 362. The cam track 370 may be engaged with components of the iris assembly 380 (described in greater detail below) which are fixed to the housing assembly 310, such that the large gear assembly 360 is effectively suspended within the housing assembly 310, allowing the large gear 362 to rotate without being hindered by contact with components of the housing assembly 310.

Still referring to FIG. 13B, the teeth of pinion 342 intermesh with the teeth of large gear 362 such that rotation of the handle 322 in one rotational direction causes rotation of the large gear 362 in the opposite rotational direction. In some examples, by providing two gears, a mechanical advantage may be obtained. For example, the size of the pinion gear 342 and the size of the large gear 362 may be selected to obtain a desired gear ratio. In one example, the gear ratio may be 2:1. In another example, the gear ratio may be 3:1. For example, the outer diameter of the pinion 342 may be about half of the outer diameter of the large gear 362, or about one-third the diameter of the large gear 362. This mechanical advantage may make it easier for the user to manually rotate the handle 322 to cause crimping of the device placed within the iris assembly 380. For example, with a 2:1 gear ratio, each turn of the large gear 362 will correspond to about two turns of the pinion gear 342, or with a 3:1 gear ration, each turn of the large gear 362 will correspond to about three turns of the pinion gear 342. Although 2:1 and 3:1 are provided as examples of possible gear ratios, it should be understood that sizes of the pinion gear 342 and large gear 362 may be selected to obtain any useful mechanical advantage (e.g. in which more than one revolution of the pinion gear 342 is required to achieve a full revolution of the large gear 362). This mechanical advantage is absent from prior art crimping devices, and may provide an easier experience for the user while crimping a device.

Although the mechanical advantage described above may be desirable in crimper 300, it may result in other items that need consideration. One consideration is the amount of absolute revolutions of the pinion gear 342 that will be required to crimp a device. For example, while a mechanical advantage achieved with a gear ratio of greater than 1:1 may be desirable, if the gear ratio is too large, it may take so many turns of the handle 322 to crimp a device such that the process takes longer than desired and/or becomes tedious. Thus, although mechanical advantage is desirable, using a gear ratio of greater than 3:1 or greater than 5:1 or greater than 15:1 may be undesirable. Thus, in some examples, the mechanical advantage should be no more than about 15:1.

Another consideration that comes into play by using two (or more) gears to achieve mechanical advantage is the placement of the gears, and in particular any gear that does not directly drive the iris assembly 380, as well as the associated handle assembly 320. For example, prior art crimpers include a single lever that drives in iris mechanism without any mechanical advantage. In such prior art crimpers, the single lever may ride along a path that rotates about the longitudinal center of the iris mechanism, and the total extent of rotation of that single lever may be about half a full revolution (such as between about 40% and about 60% of a full revolution). In these prior art designs, the single lever may extend directly radially outwardly from the iris mechanism (or from components that drive the iris mechanism open and closed). This may result in the single lever always being positioned outside of the view of the iris mechanism (and any device positioned within the iris mechanism). This prior art design may also make it simple for the single lever to have a path of travel that does not risk that the single lever contacts the table on which the crimper sits (or otherwise contacts a base of the crimper) over the entire range of motion of that single lever.

By introducing the additional complexity of one or more additional gears to achieve mechanical advantage, new problems arise that are neither seen nor appreciated in the prior art crimper devices described directly above. One such problem is that the pinion gear 342, and thus handle 322, may need to undergo more total rotation than typical of the single lever of prior art crimpers. For example, because the handle 322 may need to rotate one full revolution or more, it may need to be positioned on one side of the housing assembly 310. One issue that may result is the force on the handle tending to tip the crimper 300 since the handle 322 is on one side of the housing assembly 310. This problem may not occur in prior art crimpers because the lever in those crimpers extends directly radially outwardly from the iris mechanism, avoiding unbalanced forces being placed on the crimper 300 during actuation. As described above in connection with FIG. 12D, this may be mitigated by having the main components of the crimper positioned closer to one side of the base so that the handle is positioned closer to a center of the base.

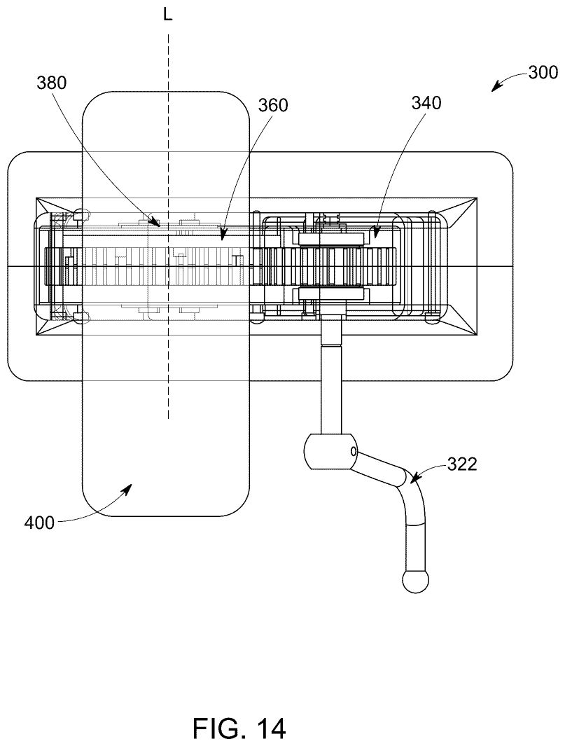

Another problem that may result from the introduction of components that provide mechanical advantage is visual obstruction of the device within the iris assembly 380. For example, if the device being used with crimper 300 is a prosthetic heart valve that is being crimped onto a balloon of a delivery device, it may be desirable to visually observe the prosthetic heart valve (or other device) as it is being crimped by the iris assembly 380. At least in part because of the placement of the handle assembly 320 to one side of the crimper 300, the handle 322 (and/or the user's hand that is grasping the handle), at least during some portion of the path of rotation of the handle during crimping, may tend to obstruct a good view of device positioned within the iris assembly 380, which would be undesirable. For example, referring briefly to FIG. 14, a top view of crimper 300 is shown, including the position of a central longitudinal axis L of the iris assembly 380, which also represents where a device (e.g. a prosthetic heart valve) would be positioned within the iris assembly 380 during crimping. It would be preferably that handle 322, during most or all of its range of rotation, avoids a visual interference zone 400 in which the handle 322 (or the user's hand gripping the handle 322) might hinder observation of the device while being crimped. The position of the pinion gear assembly 340 both above and to the side of the central longitudinal axis L of the iris assembly 380, as well as the geometry of the handle assembly 320 (and/or handle 322), may allow the handle 322 to undergo 360 degrees of rotation without entering the visual interference zone 400. In some examples, the visual interference zone 400 take the shape of a cylinder that is coaxial with the central longitudinal axis L. It should be understood that the visual interference zone 400 shown in FIG. 14 may not be to scale, but is rather intended to illustrate the concept. In some examples, the visual interference zone 400 may be defined as a cylinder having a central longitudinal axis that is coaxial with the central longitudinal axis L of the iris assembly 380, with a radius of that cylinder being between about 10 mm and about 40 mm, including between about 20 mm and about 30 mm, including about 25 mm.

Another potential issue that can arise as a result of the handle 322 undergoing a larger amount of revolution during the crimping process compared to the prior art devices is the potential for a portion of the handle 322 (such as the first handle end 324) or for a user's hand gripping the handle 322 to strike either the base 316 or the surface (e.g. table) on which the crimper 300 is resting. In order to avoid this result, the center of the pinion 342, and also position of the pinion drive shaft 344, is positioned high up on the crimper 300 (e.g. above the level of the longitudinal axis L of the iris assembly 380), and the handle 322 has a radial extent (e.g. total length between the first handle end 324 and the second handle end 328) that is smaller than the distance between the pinion drive shaft 344 and the top surface of the base 316. For example, referring to FIG. 13A, there may be a first distance D1 between the pinion drive shaft 344 (and/or the longitudinal center of the pinion 342) and the top surface of the base 316, and the handle 322 may have a second distance D2 between a center of the second handle end 328 (and/or the center of the pinion drive shaft 344) and the outer radial extent of the first handle end 324. The second distance D2 is preferably smaller than the first distance D1 in order to ensure that the handle 322 does not strike the base 316 during rotation. Preferably, the first distance D1 is larger than the second distance D2 by at least about 2 inches (or at least about 5 cm) so that a user's hand gripping the handle 322 is not likely to strike either the base 316 or the surface (e.g. table) on which the crimper 300 sits when rotating the handle 322 through the bottom of the arc along which the handle 322 revolves. It should be understood that, although a crank-style handle 322 is shown and described herein, other types of handles, including for example knob-style handles, may be used in place of any of the crank-style handles described herein.

Another item that may need to be considered as a result of the handle 322 undergoing a larger amount of revolution during the crimping process compared to the prior art devices is where the handle 322 is positioned within its path of revolution at different time points during the crimping process, such when the iris assembly 380 is completely open (e.g. prior to beginning the crimping process) and particularly when the iris assembly 380 is at a minimum size (e.g. at the point in the crimping process in which the device inside the iris assembly 380 is crimped to the minimum diameter). In prior art devices that do not have mechanical advantage, a lever may have two endpoints separated by between about 90 and about 180 degrees of rotation, resulting in little to no ability to modify where the start and end points of the lever are for the crimping process. For example, during the crimping process, the user may rotate the handle 322 through one or more down strokes (in which the second handle end 324 is getting closer to the base 316) and through one or more up strokes (in which the second handle end 324 is moving farther away from the beast 316). The crimping process, particularly when the device within the iris assembly 380 is nearing its minimum diameter (e.g. near the end of the crimping process), may require non-negligible forces supplied by the user manually on the handle 322, even with mechanical advantage. As a result, a user may find it easier to supply these larger forces as the handle 322 is on a downstroke (e.g. because the user can use his or her bodyweight to assist the downward movement of the handle 322) compared to when the handle 322 is on an upstroke. Thus, in some embodiments, the pinion 342 and large gear 362 may be keyed to each other so that, when the gears 342, 362 have rotated to place the iris assembly 380 in or near its minimum diameter (more details of this process are provided below), the handle 322 is on a downstroke.

Now that a number of issues unique to the provision of mechanical advantage in crimper 300 have been addressed, the way in which rotation of the handle 322 causes crimping of the device within the iris assembly 380 is described below. As noted above, rotating the handle 322 in one rotational direction causes the pinion 342 to rotate in the same rotational direction, and meshing of the pinion 342 and the large gear 362 results in the large gear 362 rotating in the opposite rotational direction as the handle 322.

Referring to FIG. 13B, a portion of the iris assembly 380 is shown with the iris assembly 380 in a closed or substantially closed condition in which an opening defined by a plurality of crimping members 382 (which may also be referred to as contact member or crimping jaws) is at a minimum. In the particular illustrated example, the iris assembly 380 includes a total of twelve contact members 382, although it should be understood that more or fewer may be provided. Each contact member 382 may include a main body 384 which terminates in a contact arm 386 which may extend at an angle relative to the main body 384. The plurality of contact arms 386, in the aggregate, define the opening of the iris assembly 380, with the opening of the iris assembly 380 being relatively small when the contact arms 386 are positioned relatively close to the longitudinal axis L of the iris assembly 380, and being relatively large when the contact arms 386 are positioned relatively far from the longitudinal axis L of the iris assembly 380. It should be understood that it is the exposed surface of the contact arms 386, which are all oriented generally facing the longitudinal axis L of the iris assembly 380, which contact the device within the iris assembly 380 during crimping.

Still referring to FIG. 13B, it should be understood that the contact members 382 of the iris assembly 380 may all be positioned radially within the portion of the large gear 362 that from the gear teeth that mesh with corresponding gear teeth of pinion 342. Each contact member 382 may include at least one protrusion 388 extending therefrom, in a direction generally parallel to the longitudinal axis L of the iris assembly 380. In the illustrated example, each contact member 382 includes two protrusions 388 that are aligned with each other along the same axis, although in FIG. 13B only one protrusion 388 is visible per contact member 382. Preferably, the protrusions are each generally cylindrical or pin shaped. Each protrusion 388 is preferably fixed to the correspond main body 384 of the contact member 382, for example by being formed integrally with the contact member 382, by being adhered to the contact member 382, by being welded to the contact member 382, or by any other suitable mechanism to achieved a fixed relationship.

Although each of the contact members 382 may be substantially identical to each other, groups of the contact members 382 may be provided with their protrusions 388 at slightly different positions. For example, in the specific embodiment of FIG. 13B, a total of twelve contact members 382 are provided in four groups, represented by the “1,” “2,” or “3” printed on the contact member 382 in FIG. 13B. The contact members 382 labeled “1” may include a protrusion 388 relatively far from the contact arm 386, and the contact members 382 labeled “3” may include a protrusion 388 relatively close to the contact arm 386, with the contact members 382 having a protrusion 388 at an intermediate location compared to the other two groups of contact members 382. As should be understood below with the description of cam track 370, this configuration may allow for all protrusions 388 to be received within different positions on a spiral groove 372 of the cam track 370 while maintaining a generally circular opening defined by the contact arms 386.

FIGS. 13A-B also show a first crimping guide 390, which may be part of the iris assembly 380, positioned behind the plurality of contact members 382. The first crimping guide 390 may be positioned generally radially inward of the outer portions of the large gear 362, and the first crimping guide 390 may be fixed to a portion of the housing assembly 310 (e.g. rear shell 312 and/or front shell 314). In some embodiments, a pair of first crimping guides 390 may be provided, such that one first crimping guide 390 is positioned on each side of the plurality of contact members 382. For example, referring now to FIG. 13C, another first crimping guide 390 is shown positioned in front of the plurality of contact members 382. The first crimping guide(s) 390 may be generally circular or cylindrical, and may each define a center opening that has a center axis that is substantially coaxial with the longitudinal axis L of the iris assembly 380. With this configuration, the interior opening of the iris assembly 380 is accessible through the corresponding central opening in the first crimping guide(s) 390. As shown in FIG. 13C, the first crimping guide(s) 390 may include a plurality of radial slots 392 formed therein. Preferably, the first crimping guide(s) 390 have the same number of radial slots 392 as the total number of contact members 382, which is twelve in this particular embodiment. Each radial slot 392 may have a width that is about equal to or slightly larger than the width or diameter of the protrusion 388, and a length that is greater than the width. Preferably, each slot 392 includes a fully enclosed boundary defined by the first crimping guide 390, with the slot 392 extending through an entire thickness of the first crimping guide 390. The longer dimension of each slot 392 (e.g. the length dimension) may extend generally radially toward and away from the radial center of the first crimping guide 390. With this configuration, as described in greater detail below, the protrusion(s) 388 of each contact member 382 may extend through a corresponding slot 392 of the first crimping guide(s) 390. If a pair of first crimping guides 390 is provided, each contact member 382 may include a pair of oppositely extending protrusions 388 extending through corresponding slots 392 of the first crimping guides 390. Whether one or two first crimping guide(s) 390 are provided, the slots 392 may prevent any side-to-side motion of the contact members 382 via the close fit of each protrusion 388 within the width of the corresponding slot 392. As is described in greater detail below, when the second crimping guide or cam track 370 forces the contact members 382 to move radially inwardly or outwardly toward the longitudinal axis L of the iris assembly 380, the slots 392 may both guide and limit the extent of the movement of the contact members 382, via the interaction between each protrusion 388 and its corresponding slot 392. As noted above, whether one or two first crimping guides 390 are provided, each first crimping guide 390 is fixed to the housing assembly 310 so that each first crimping guide 390 remains stationary relative to the housing assembly 310, including when the handle 322 forces the pinion 342 and large gear 362 to rotate.

Referring now to FIG. 13D, crimper 300 may include a second crimping guide 370, which may also be referred to as a cam track 370. In some embodiments, a pair of cam tracks 370 are provided, one on each side of the plurality of contact members 382. The cam track 370 may be thought of as part of the large gear assembly 360. In some embodiments, the cam track(s) 370 is provided outward of the first crimping guide 390. In other words, if two first crimping guides 390 are provided, they may both be positioned between a pair of cam tracks 370. Generally, the cam track(s) 370 may be substantially circular or cylindrical, with an inner opening that generally aligns with the opening of the iris assembly 380 when the crimper 300 is fully assembled so that the device to be crimped may be placed into the interior of the iris assembly 380 through corresponding openings in the housing assembly 310, the cam track(s) 370, and the first crimping guide(s) 390. As best shown in FIGS. 13A and 13D, each cam track 370 may include a plurality of spiral-shaped grooves 372 formed therein. Each spiral-shaped groove 372 may have a first terminal end positioned relatively close to the outer radial edge of the cam track 370, and may extend in a spiraling fashion around the central opening of the cam track 370 to a second terminal end that is positioned relatively close to the central opening of the cam track 370. In other words, the radial position of each groove 372 gradually moves toward the central opening of the cam track 370 from the first terminal end of the groove 372 toward the second terminal end of the groove 372.

The number of grooves 372 provided in the cam track 370 may relate to the number of contact members 382. For example, as noted above, the illustrated example includes a total of twelve contact members 382, arranged as four groups of three contact members 382 per group. Thus, with this configuration, a total of four spiral-shaped grooves 372 may be provided corresponding to the four groups of three contact members 382. The protrusion(s) 388 of each contact member 382 in a single group may extend into the same groove 372, with the different heights or positions of the protrusion(s) 388 in each contact member 382 of the group allowing for the protrusion(s) 388 to fit within different portions of the spiral-shaped groove 372 while the contact members 382 are otherwise in an identical radial position. As should be understood, this positioning may be repeated for each group of contact members 382 for the remaining spiral-shaped grooves 372. Further, it should be understood that each spiral-shaped groove may have a width that is about equal, or slightly larger than, the width or diameter of the protrusion(s) 388, similar to the width of the radial slot(s) 392. The protrusion(s) 388 may have a length that allows them to first pass through the corresponding radial slot(s) 392, and then into the corresponding spiral-shaped groove 372.

Still referring to FIG. 13D, the cam track(s) 370 may be fixed to the large gear 362. For example, fasteners may pass through an outer peripheral portion of the cam track(s) 370 and into a location at an outer periphery of the large gear 362, such that the large gear 362 and the cam track(s) 370 rotate in unison. In some examples, a single cam track 370 is provided, and in other examples, as noted above, a pair of cam tracks 370 may be positioned on each side of the plurality of contact members 382.

In an exemplary use of crimper 300, the iris assembly 380 may have an initial configuration in which the opening of the iris assembly 380 is maximally open, which may correspond to each of the contact members 382 (and particularly contact arms 386) being positioned their maximum radial distance away from the longitudinal axis L of the iris assembly 380. This position also corresponds to the protrusion(s) 388 of each contact member 382 being at their farthest position along radial slots 392 away from the longitudinal axis L of the iris assembly 380, and the protrusion(s) 388 being at their position along spiral-shaped grooves 372 as close to the first terminal end of the spiral-shaped groove 372 as the configuration allows (e.g. it should be understood that the protrusion 388 of each contact member 382 will be at a different position relative to the first terminal end of the corresponding spiral-shaped groove 372 in this initial position). With the crimper 300 in this initial state with the iris assembly 380 fully (or nearly fully) open, a user may place the device to be crimped within the interior of the opening of the iris assembly 380. In some examples, the device is a balloon-expandable prosthetic heart valve that is in an expanded condition and which is positioned over an inflated balloon of a delivery device.

When the user is ready to start crimping the device within the open iris assembly 380, the user need only rotate the handle 322 in the correct direction. Rotating the handle 322 causes the pinion 342 to rotate in the same direction, which forces the large gear 362 to rotate in the opposite direction. Rotation of the large gear 362 forces the cam track 370 to rotate in unison. As the cam track 370 rotates, the contact members 382 remain rotationally stationary at least due to the protrusions 388 being confined within respective radial slots 392 of the first crimping guide 390, the first crimping guide 390 being fixed to the housing assembly 310. Thus, as the cam track 370 rotates, the spiral grooves 372 rotate, forcing the protrusions 388 within the spiral grooves 372 to move radially inwardly, with that radial inward movement being guided and limited by the corresponding radial slot 392 which remains stationary. The rotation of the cam track 370 thus causes all of the contact members 382 to move radially inwardly in unison to reduce size of the opening of the iris assembly 380, with the assembly of contact arms 386 maintaining a generally circular or cylindrical shape that reduces in diameter as the cam track 370 rotates. The contact arms 386 press on the device within the opening of the iris assembly 380 as the contact arms 386 move radially inwardly, applying force on the device that causes the device to collapse or crimp. In the case of a balloon-expandable prosthetic heart valve, as the contact arms 386 are forced radially inwardly, the frame of the prosthetic heart valve will begin to collapse, as will the balloon positioned within the prosthetic heart valve as inflation media (e.g. saline) is forced out of the balloon due to the crimping forces. As noted above, preferably, as the user manually turns the handle 322, neither the handle nor the user's hand are in danger of striking either the base 316 or the surface on which the base 316 sits. The user may continue rotating the handle 322 to further crimp the device until the device reaches a desired minimum diameter, which preferably occurs (based on the starting configuration of the crimper 300) when the handle 322 is on a downstroke (e.g. moving at least partially in the direction of gravity toward the base 316). Further, as noted above, during the crimping, the handle 322 may be entirely or substantially entirely out of the visual interference zone 400 (refer to FIG. 14) to maximize the ability of the user (or someone associated with the user) in viewing the device (e.g. the prosthetic heart valve) as it collapses.

In some examples, the user may rotate the handle 322 until, based on visual or other observation, the device within the iris assembly 380 has reached the desired diameter. However, in other examples, one or more mechanical stops may be provided in order to stop the crimping process at a certain point, for example at a set desired minimum diameter of the opening of the iris assembly 380, which may help prevent over-crimping which could damage the device (e.g. a prosthetic heart valve) within the crimper 300, or even damage components of the crimper 300 itself.

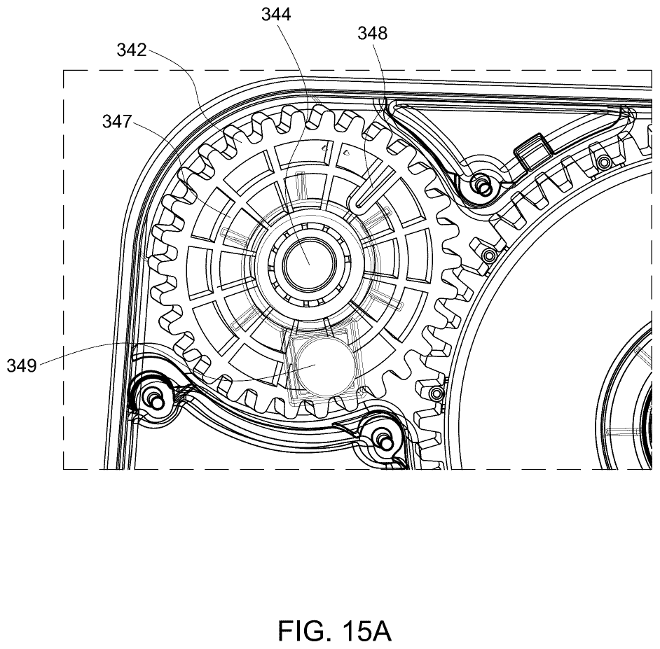

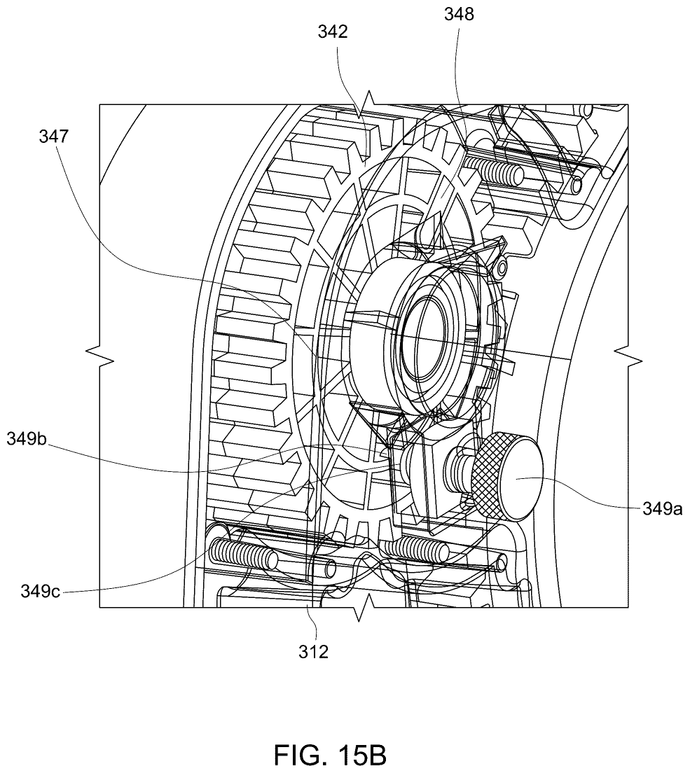

FIGS. 15A-B illustrate one example of a positive stop mechanism that may be implemented to prevent over-crimping of a device. In particular, FIGS. 15A-B are front and perspective views, respectively, of the section of the crimper 300 that includes the pinion assembly 340, with the rear shell 312 being shown in partial phantom for clarity of illustration. As shown in FIGS. 15A-B, pinion 342 may include a substantially circular rim 347 positioned between the gear teeth at the outer periphery of the pinion 342 and internal opening which receives the pinion drive shaft 344. The rim 347 may provide a surface against or along which a portion of a spring pin 349 may slide as the pinion 342 rotates (and while the spring pin 349 remains stationary). The rim 347 may be substantially circular but for an interruption in the rim 347 in the form of a groove, recess, or detent 348 sized and shaped to receive a portion of the spring pin 349.

Referring in particular to FIG. 15B, spring pin 349 may include a head portion 349a positioned outside the rear shell 312, which may be enlarged relative to a pin portion 349b that is coupled to the head portion 349a and which extends through the rear shell 312 into contact with the rim 347. The head portion 349a may be knurled or have other features to enhance a user's ability to grip the spring pin 349. A biasing member, such as a spring 349c, may be operatively coupled to the spring pin 349 and in a compressed condition when the pin portion 349b contacts the rim 347. For example, a flange or other surface may be provided on the pin portion 349b, with the spring 349c having one surface in contact with the interior of the rear shell 312 and another surface in contact with the flange, so that the spring 349c tends to push the pin portion 349b into contact with the rim 347. As the user operates the crimper 300 to crimp a device within the iris assembly 380, the pin portion 349b remains in forcible contact with the rim 347 as the pinion 342 rotates, until the pin portion 349b is in alignment with the pin detent 348. Once the pinion 342 has been rotated enough so that the spring pin 349 is in alignment with the pin detent 348 of the pinion 342, the spring 349c is able to decompress, forcing the pin portion 349b to drive into the space of the pin detent 348. With the pin portion 349b within the detent 348, the pinion 342 is unable to rotate any further as contact between the spring pin 349 and the pinion 342 prevents such rotation. If a user prefers to override the spring pin 349 to either further crimp the device within the iris assembly 380, or to rotate the pinion 342 in the opposite direction to open the iris assembly 380, the user may simply pull on the head 349a to compress the spring 349c and remove the pin portion 349b from within the pin detent 348. Once rotation of the pinion 342 starts, the user may release the grip on the head 349a which will tend to push the pin portion 349b back into contact with the rim 347 as rotation of the pinion 342 continues. Because the rotational orientation of the pinion 342 has a defined relationship to the radial position of the contact members 382 (and thus the size of the opening of the iris assembly 380), the pin detent 348 may be positioned at a rotational position along the rim 347 that corresponds to the desired minimum crimping diameter of the device being crimped with crimper 300 (e.g. by corresponding to the desired minimum diameter of the opening of the iris assembly 380 defined by the radial position of the contact arms 386).

In the embodiments above, the handle assembly 320 is described as having a second handle end 328 that is rotationally fixed (e.g. via keyed geometry) to a pinion drive shaft 344 so that rotation of the handle 322 rotates the pinion drive shaft 344 which rotates the pinion 342. In other embodiments, instead of a separate pinion drive shaft 344, the handle assembly 320 may include a pinion drive shaft that is formed integrally with the handle 322, for example extending from the second handle end 328. In still other examples, instead of the second handle end 328 being rigidly keyed to the pinion drive shaft 344, the handle 322 may have a ratcheting engagement with the pinion drive shaft 344. For example, the pinion drive shaft 344 and the second handle end 328 may each be generally cylindrical, with the pinion drive shaft 344 and/or the interior of the second handle end 328 having directional teeth. If this ratcheting mechanism is included, rotation of the handle 322 in the rotational direction to close the iris assembly 380 may be fully transmitted to the pinion drive shaft 344, the pinion 342, and the large gear 362 to close the iris assembly 380. However, rotation of the handle 322 in the opposite rotational direction may not produce any rotation of the pinion drive shaft 344. This ratcheting mechanism may have at least one advantage in that it may allow a user to fine tune the specific angle of the handle 322 at any given point during the crimping process, which may for example be useful to position the handle 322 to maximize the ability of the user to apply body weight to a down stroke of the handle 322.

Although the embodiments described above generally achieve mechanical advantage using a smaller pinion gear 342 that directly meshes with a larger gear 362 to drive the iris assembly 380 to open or close, it should be understood that mechanical advantage may be similarly obtained with other mechanical features besides two differently sized spur gears that intermesh. For example, in another example, the intermeshing large and pinion gears may be replaced with pulleys having teeth that engage a timing belt (e.g. a toothed belt) to transmit toque from the handle to ultimately open and close the iris assembly 380. Other types of gears that directly interface with each other besides or in addition to spur gears may also be used in other examples.

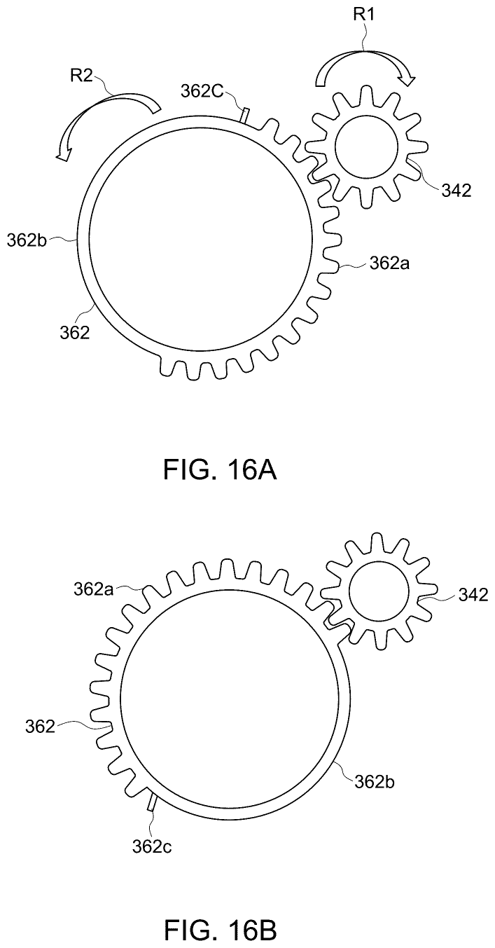

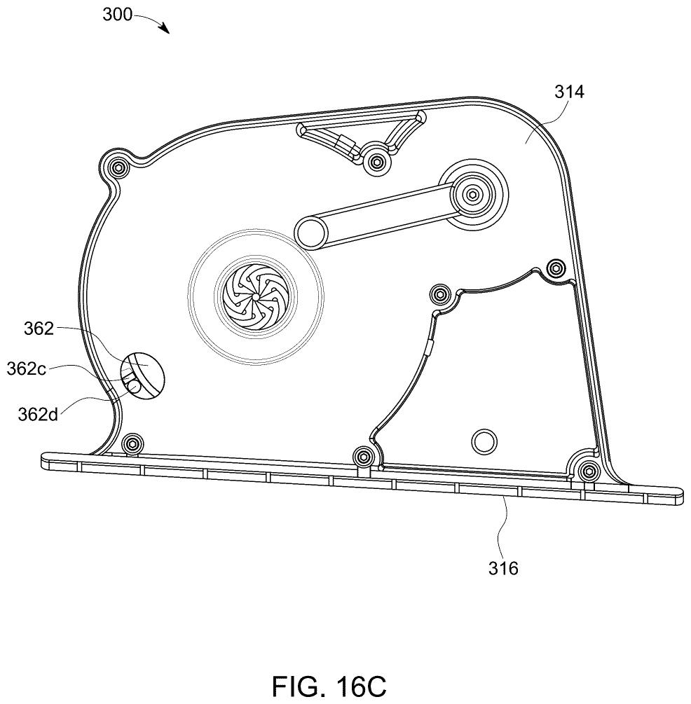

Although a hard stop mechanism on the pinion assembly 340 is shown and described in connection with FIGS. 15A-B, in some embodiments, a hard stop mechanism may instead (or additionally) be provided on the large gear assembly 360. FIG. 16A illustrates an example of the pinion gear 342 meshing with the large gear 362 at an early stage of crimping when the iris assembly 380 is mostly or entirely open. In the example of FIG. 16A, the pinion 342 is about half the diameter of the large gear 362. As with other embodiments described herein, as the pinion 342 is rotated in a first rotational direction R1, the large gear 362 is forced to rotate in a second rotational direction R2 opposite the first rotational direction to close the iris assembly 380. In some examples, the iris assembly 380 may be configured so that the entire range of motion between the iris assembly 380 being maximally open and the iris assembly 380 being at the minimum desired diameter is achievable with about a half revolution of the large gear 362, which in this example corresponds to about a full revolution of the pinion 342. However, it should be understood that other gear ratios, as well as other configurations of the iris assembly 380, may result in the large gear 362 needing to undergo a smaller or larger amount of revolutions than about a half a revolution to transition the iris assembly 380 from being at the initial desired maximum size to being at the final desired minimum size.

In examples in which the large gear 362 only need to undergo less than a full revolution, as shown in FIG. 16A, the large gear 362 may be provided with a toothed section 362a that does not extend around the entire circumference of the large gear 362, allowing a portion of the large gear 362 to remain smooth or without teeth, shown in FIG. 16A as the smooth section 362b. As shown in FIG. 16A, a hard stop 362c may be provided protruding radially outwardly from the large gear 362c ahead of the first gear tooth (e.g. in the leading direction when the large gear 362 is turning in the second rotational direction R2 to close the iris assembly 380). In some examples, the hard stop 362c may be any structure that is fixed to the large gear 362 which does not interfere with other components of the crimper 300 (other than the intended interference described below). In some examples, the hard stop 362c may be another gear tooth, which may be similar or identical to the other gear teeth on large gear 362.

FIG. 16B illustrates the gears of the crimper 300 after the iris assembly 380 has reached its minimal desired size, which in this example has been achieved via about one full revolution of the pinion 342 in the first rotational direction R1 and about a half revolution of the large gear 362 in the second rotational direction R2. It should be understood that, even though large gear 362 has gear teeth on only about half the circumference of the large gear 362, the smooth section 362b does not hinder the ability of the gears to move along their entire intended use range. However, having a smooth section 362b on the large gear 362 may create an option for using the hard stop 362c. For example, FIG. 16C illustrates a stop member 362d, which may be a pin or any other rigid member, inserted through an opening in the front shell 314. The rear shell 312 may also include a similar or identical opening. In some examples, the stop member 362d may reversibly secure to the housing assembly 310 (e.g. to the rear shell 312 and/or front shell 314) so that the stop member 362d may be removed. In some examples, the stop member 362d may be generally cylindrical and formed of metal or hard plastic. As shown in FIG. 16C, the stop member 362d may extend through at least a portion of the housing assembly 310 so that it is positioned just radially outwardly of the large gear 362 along the smooth section 362b. The stop member 362d may be positioned to correspond to the position that the hard stop 362c will be in (or adjacent to) when the iris assembly 380 has been closed to the minimum desired diameter. As a result, as the user closes the iris assembly 380 by rotating the gears, the large gear 362 will continue to rotate in the second rotational direction R2 to close the iris assembly 380 until the hard stop 362c contacts the stop member 362d. This contact prevents further rotation of the large gear 362 in the second rotation direction R2, which may help avoid damaging the device within the iris assembly 380 by accidental over-crimping, while still allowing the iris assembly 380 to be opened by reversing the direction of rotation of the gears. Further, in some situations it may be desirable to continue to reduce the iris assembly 380 to a size smaller than the pre-determined minimal size. In those cases, the user may simply remove the stop member 382d and continue rotating the large gear 362 in the second rotational direction R2, as long as enough distance remains in the toothed section 362a to mesh with the pinion 342. Although one specific example of a hard stop on the large gear 362 is shown and described in connection with FIGS. 16A-C, it should be understood that different gear ratios, different desired minimum sizes of the iris assembly 380, etc. could result in slightly different specific configurations of positions of the features that allow for the gear stop (e.g. the toothed section 362a, the smooth section 362b, the position of the hard stop 362c, and/or the position of the stop member 362d.

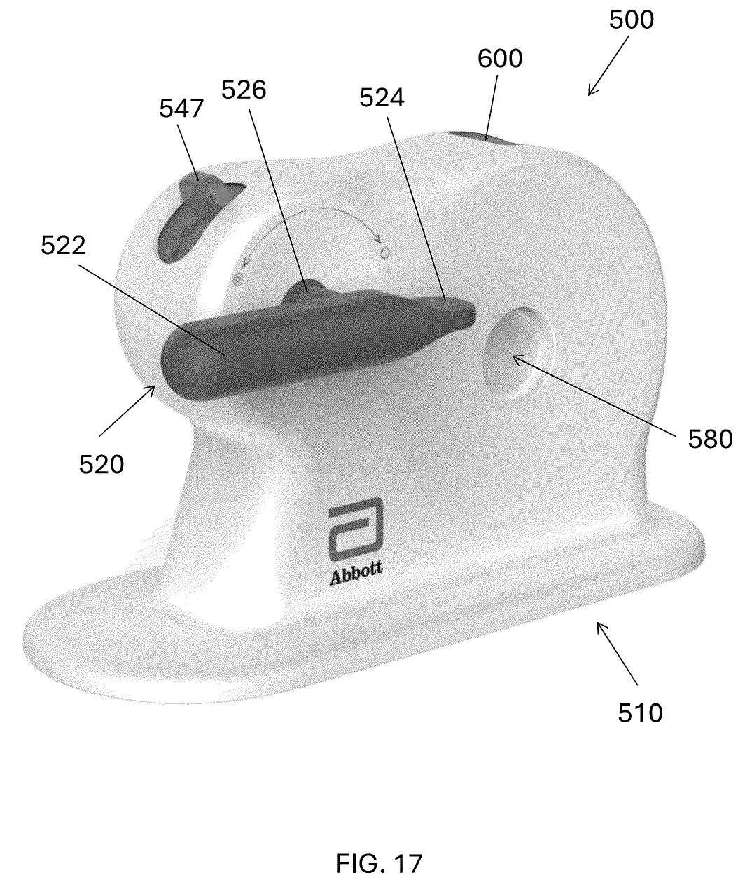

FIG. 17 illustrates another example of a crimper 500 that has many features in common with crimper 300, and it should be understood that not all features in common between crimpers 300 and 500 are described explicitly again for crimper 500. FIG. 17 shows the crimper 500 in an assembled condition and thus the internal components are not visible in FIG. 17. Generally, crimper 500 may include a housing assembly 510, an actuator or handle assembly 520, a pinion or first gear assembly 540 (not visible in FIG. 17), a large or second gear assembly 560 (not visible in FIG. 170, and an iris assembly 580. As with crimper 300, although the word “assembly” is used in connection with these components, an “assembly” in some embodiments may include either a single component or multiple components that are structurally or functionally related. On a general level, the crimper 500 may be used for the same purpose as crimper 300.

As with housing assembly 310, housing assembly 510 may include a rear shell, a front shell, and a base, which may all be similar or identical to the corresponding components of housing assembly 310 (either including an offset position, or otherwise a centered position, of the front and rear shell relative to the base).

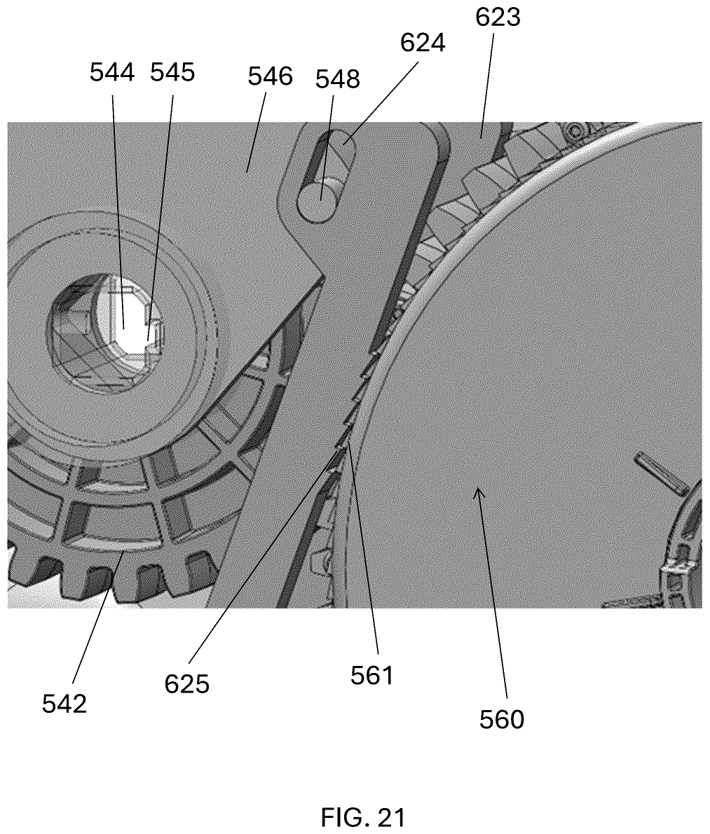

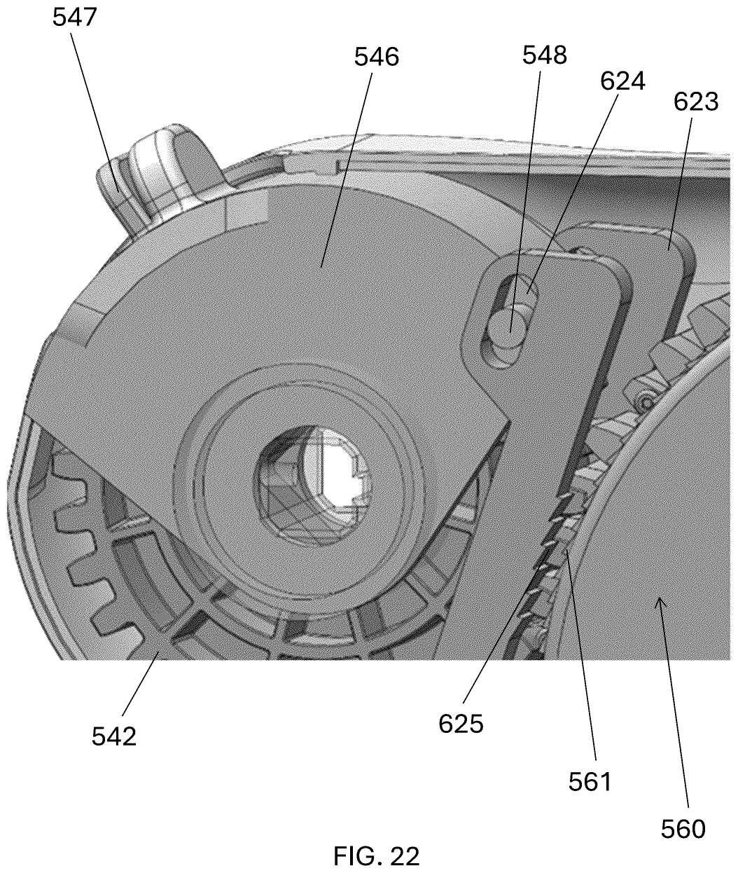

One difference between crimper 300 and crimper 500 is the configuration of the handle assemblies. For example, the handle assembly 520 of crimper 500 may be provided as an accessory that is capable of coupling to the housing assembly 510 on either side to allow for right-handed or left-handed actuation of the handle assembly. In the illustrated example, the handle assembly 520 includes a generally cylindrical ergonomic body portion 522 and a flattened end portion 524. With this configuration, the user may grip the body portion 522 comfortably with the palm, and grip the flattened end portion 524 with a thumb and index finger for rotating the handle assembly 520 during crimping. The handle assembly 520 may also include a rod 526 (only a portion of which is visible in FIG. 17) extending generally orthogonally to the body portion 522, originating at a point near the transition between the body portion 522 and the end portion 524. The rod 526 is sized and shaped to be inserted into and engage with the pinion gear assembly 540. The rod 526 may have a generally rectangular or square cross-section that is configured for receipt in a correspondingly-shaped recess in the pinion gear assembly 540 (as shown in FIGS. 21-22). With this configuration, rotation of the body portion 522 and the end portion 524 will cause corresponding rotation of the rod 526, and the keyed shapes (which may be other shapes besides rectangular or square cross-sections) will transfer torque from the rod 526 to the pinion gear assembly 540, causing corresponding rotation of the pinion gear assembly 540. The rod 526 may include additional keyed shapes, such as a protrusion that can be received in only a single orientation within a corresponding detent of the pinion gear assembly 540 (as shown in FIGS. 21-22). With this configuration, not only may the user insert the handle assembly 520 into either side of the pinion gear assembly 540 for either right- or left-handed crimping, but the protrusion on the rod 526 will ensure that the main body 522 and end portion 524 are facing in the desired direction when coupling the components. In one example, when the iris assembly 580 is in the fully open condition, the end portion 524 points forward (generally toward the iris assembly 580) with the handle assembly 520 generally parallel to the base of the housing assembly 510. During crimping, the handle assembly 520 may rotate about 360 degrees (in the anticlockwise direction in the view of FIG. 17) until the handle assembly 520 is again about in the position shown in FIG. 17.

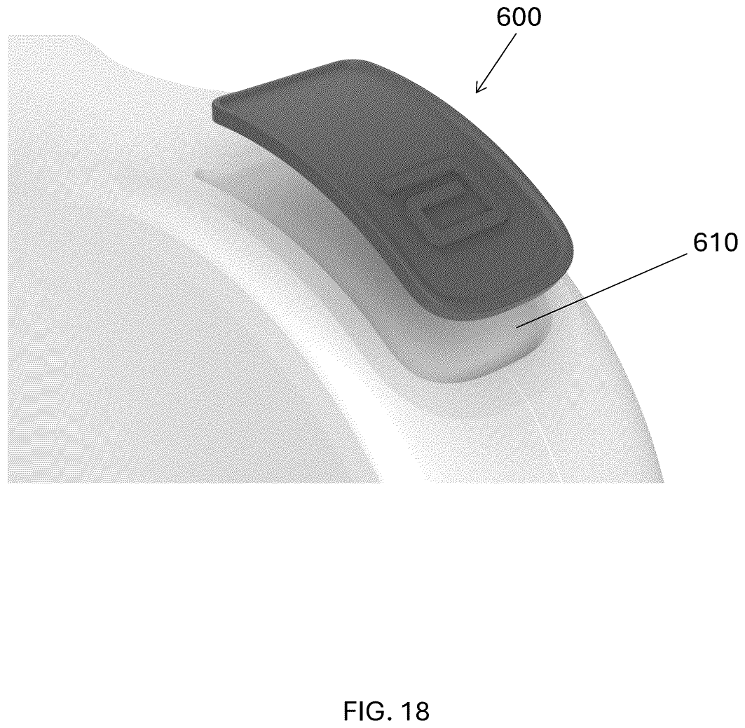

Another difference between crimper 300 and crimper 500 is the inclusion of a sizing key 600. As shown FIG. 18, the sizing key 600 may have an outer shape that generally matches the contour of the housing assembly 510, and may be configured to be received in a correspondingly shaped opening 610 in the housing assembly 510, which may be positioned for example on a top of the housing assembly 510 near the location of the large gear assembly 560. Although not shown in FIG. 18, the opening 610 in the housing assembly 510 may provide access to the large gear assembly 560 so that the sizing key 600 may limit the size to which the medical device can be crimped during crimping by providing a physical barrier against further rotation of the large gear assembly 560. It should be understood that the sizing key 600 may be provided with the medical device (e.g. the prosthetic heart valve and the delivery system), with the sizing key 600 coming in different configurations that provide different crimping limits. Thus, the user may use the sizing key 600 that comes with the particular medical device, ensuring that the sizing key 600 will help ensure the correct amount of crimping for the medical device. For example, if a prosthetic heart valve comes in three different sizes, each being intended to be crimped to a different specific size using crimper 500, the sizing key 600 may come in three different variations corresponding to the three different prosthetic heart valve sizes and three different intended crimping sizes. In such scenarios, the sizing key 600 may also be provided with indicia (e.g. numbers, symbols, colors, etc. to indicate the intended crimping size or the nominal size of the medical device). Such indicia may provide the user additional comfort that the sizing key 600 is the correct sizing key for the particular medical device being crimped.

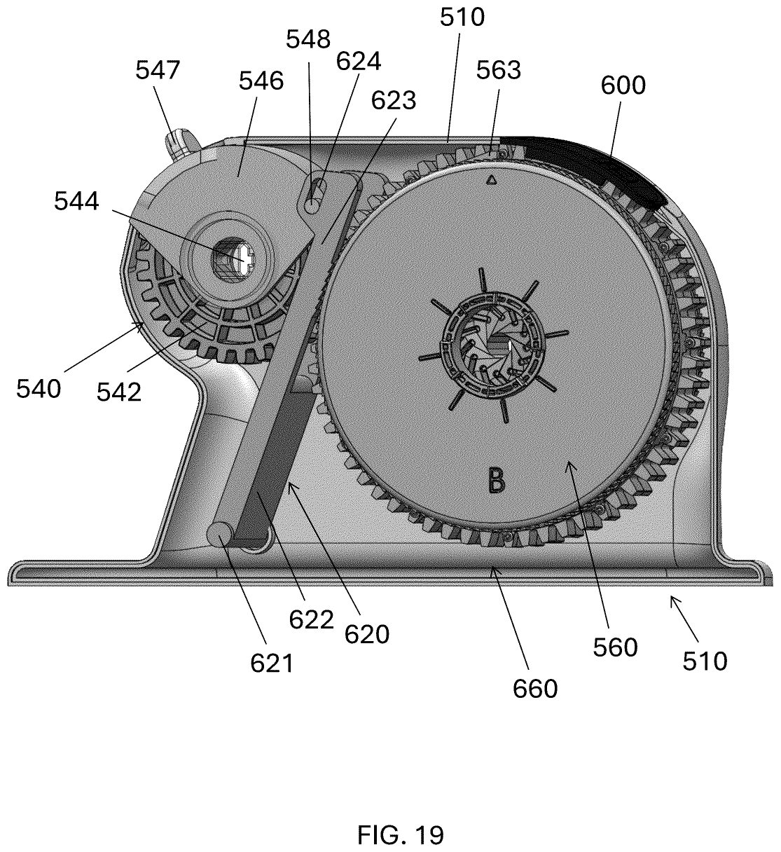

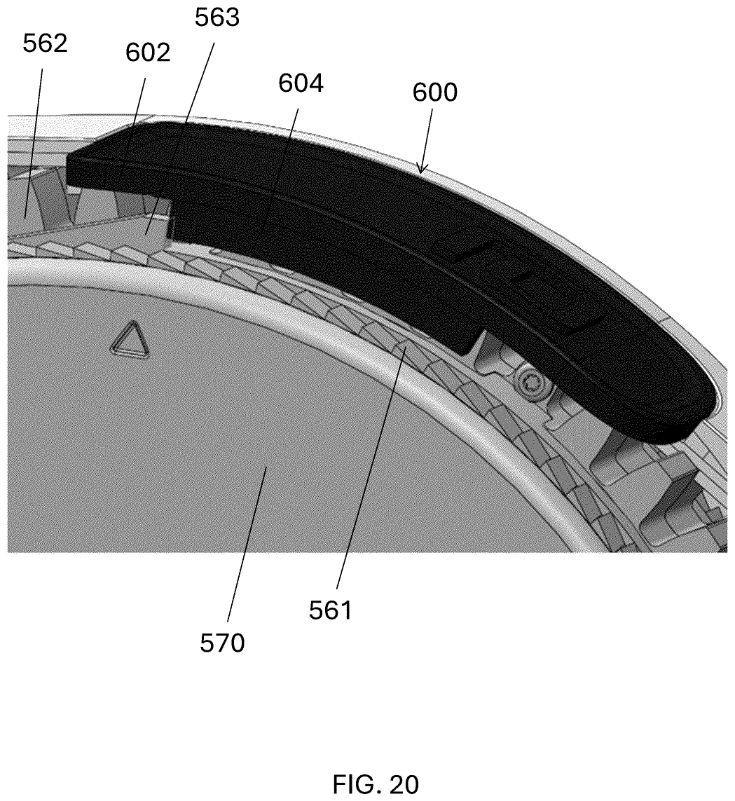

FIG. 19 illustrates crimper 500 with a portion of the housing assembly 510 removed, and the handle assembly 520 having been removed, to better illustrate a number of components internal to the crimper 500. Generally, FIG. 19 shows the pinion gear assembly 540, the large gear assembly 560, a crimping direction limiter (which may include an actuator 546 an interference mechanism 620 described in greater detail below), and the sizing key 600 assembled to the housing assembly 510. FIG. 20 is an enlarged view of the portion of FIG. 19 showing the sizing key 600. The sizing key 600 may include an outer or top portion 602 which generally matches the size and the shape of the opening 610, and the outer or top portion 602 may be substantially identical among the different sizing keys 600 corresponding to the different intended crimp sizes. The sizing key 600 may also include an inner or bottom portion 604 extending into the interior of the crimper 300. The inner or bottom portion 604 may include two extensions (only one visible in FIG. 20) that have a space between them to allow the teeth of the large gear 562 to pass through without interruption. In other words, a track or channel may be formed between the two extensions that form the bottom portion 604 along which the teeth of the large gear 562 may pass without interference. The large gear assembly 560 may include a pair of stops 563 (only one visible in FIG. 21, and in some embodiments only a single stop 563 may be included) that is positioned radially outward of the teeth of the large gear 562. The stop 563 may be formed as part of the large gear 562 itself, or on any of the rotating components of the large gear assembly 560, including the cam tracks (which may be substantially identical to cam track 370, including spiral grooves 372). As the large gear 562 and the cam tracks rotate during crimping, the stop 563 also rotates and is brought closer to the sizing key 600, until a flat surface of the stop 563 contact the inner or bottom portion 604 of the key 600, as shown in FIG. 20. This contact between the stop 563 and the sizing key 600 prevents further rotation of the large gear 562, and thus may be used to set a maximum amount of rotation that the large gear 562 may go through before hitting the sizing key 600. As noted elsewhere, different sizing keys 600 may be created to correspond to different minimum crimp sizes, and the part of each sizing key 600 that may be different to achieve the different sizes is the extent of the inner or bottom portions 604 of the key 600. For example, in the view of FIG. 20, the position of the left end of the inner or bottom portion 604 may be changed to change the point at which the stop 563 contacts the sizing key 600 to stop further rotation. If the left end of the inner or bottom portion 604 is adjusted to the right in FIG. 20, the large gear 562 would be able to go through more rotation before the stop 563 contacts the sizing key 600, allowing for a tighter crimp. These dimensions of the inner or bottom portion 604 can be tailored to the amount of rotation of the large gear 562 needed to achieve the minimum crimp profile of the medical device that is desired. It should also be understood that, if a user determines that a tighter crimp is needed than the available sizing key 600 allows, the sizing key 600 could be removed from the crimper 500 to allow the large gear 562 (and associated stop 563) to rotate further for additional crimping without interference.

A further difference between crimper 300 and crimper 500 is the inclusion of a crimping direction limiter in crimper 500 which may help prevent actuation of the handle assembly 520 in the wrong direction. Referring back to FIG. 19, the pinion gear assembly 540 may include a pinion 542 which may be similar or identical to pinion 342, including being formed as a spur gear with a plurality of pinion teeth extending radially outwardly from an outer circumference thereof. The pinion 542 may define a central opening 544 that has a shape that is geometrically (or otherwise) keyed to a portion of the handle 522 that is inserted into the opening 544. Referring briefly to FIGS. 21-22, which show enlarged views of portions of these internal components, the generally square opening 544 is visible along with the detent 545 that receives the corresponding protrusion in the rod 526 of the handle assembly 520 to ensure that the handle assembly 520 can only be coupled in one orientation (for each of the right-handed and left-handed connection options) to the pinion gear assembly 540. This opening 544 may be formed directly in the pinion gear 542 (e.g. the central opening of the pinion gear 542 itself may have this shape), or in other examples, an insert may be provided with this shape, the insert having an outer shape that is keyed to an internal shape of the pinion gear 542 to allow for transmission of torque.

Referring to FIGS. 19 and 21-22, the crimping direction limiter may include an actuator 546. The actuator 546 in the illustrated example is a cover that covers a limited portion of the pinion 542 and which does not directly affect the movement of the pinion 542. The actuator 546 may include a main body which forms a portion of a hollow circular member that overlies a portion of the pinion 542, although the portions of the pinion 542 that interface with the large gear assembly 560 are not covered by the actuator 546. The actuator 546 may also include a button, knob, lever, or tab 547 (although any other structure for gripping can be used instead). As best shown in FIG. 17, the tab 457 protrudes through an opening in the housing assembly 510 near the pinion assembly 540 and the handle assembly 520. The actuator 546 also includes a pair of pins 548 projecting toward the two sides of the housing assembly 510 (although in some instances a single pin may be used, and only one pin is visible in the figures). As is explained in greater detail below, pulling or pressing the tab 547 downward from the top of the recess in the housing assembly 510 (the position shown in FIG. 17) causes the actuator 546 to rotate slightly in the anti-clockwise direction in the views of FIG. 21-22, pulling the pins 548 through an arc-shaped movement.

Referring back to FIG. 19, the crimping direction limiter may also include a pawl or interference mechanism 620. The interference mechanism 620 may include a pair of pins 621 at a first end, with each pin 621 being received in a corresponding opening formed in the interior of the sides of the housing assembly 510 (although only one of these two corresponding openings is visible in the view of FIG. 19). With this configuration, the interference mechanism 620 is rotatable or articulable about a longitudinal axis passing through the pins 621. The interference mechanism may include a solid body 622 closer to the pins 621, transitioning into two side walls 623 with an opening therebetween toward the opposite end of the interference mechanism 620. It should be understood that the interference mechanism 620 may omit the solid body 622 or otherwise have a smaller extent for the solid body 622 than shown. As shown in FIG. 19, the space between the two side walls 623 is sufficient to allow the teeth of the large gear 562 to mesh with the teeth of the pinion gear 542 through that space. As best shown in FIGS. 21-22, each of the side walls 623 may include an oval or stadium-shaped recess 624 near the end of the side walls 623 opposite the pins 621. The recesses 624 have a width that generally corresponds to the diameter of the pins 548, which are received in the recesses 624. The recesses 624 also have a length that is greater than the diameter of the pins 548, so that the pins 548 are capable of moving up and down along the recesses 624. With this configuration, as the tab 527 is pulled downward from the lock position shown in FIG. 17 to an unlocked position, the actuator 546 (including the pins 548) rotate in an anti-clockwise direction in the views of FIGS. 21-22, with the pins 548 moving up the recess 624 and causing the interference mechanism 620 to also rotate in the anti-clockwise direction about pins 621. The position of the interference mechanism 620 while the tab is in the locked condition is shown in FIG. 21, and the position of the interference mechanisms 620 after the tab has been moved down away from the locked condition is shown in FIG. 22 (note that only the tab 547 with the label represents the unlocked condition of the tab 547, while the unlabeled tab in FIG. 22 represents the prior locked position). The side walls 623 of the interference mechanism 620 may each include a plurality of ratchet teeth 625 (best shown in FIGS. 21-22) along the surfaces of the side walls that confront the large gear assembly 560. The ratchet teeth 625 may have an orientation in which the ramped surface of the ratchet teeth 625 generally extend from the side walls 623 toward the top (opposite the pins 621) of the interference mechanism 620 and toward the large gear assembly 560, and then sharply back away from the large gear assembly 560. As is described immediately below, the movement of tab 547 (and thus interference mechanism 620) may engage or disengage the ratchet teeth 625 that control the direction(s) in which the large gear 562 may rotate. In some examples, a biasing member (e.g. a spring) may be provided to push the tab 547 toward the locked or up condition shown in FIG. 17, although in other examples such a biasing member may be omitted.

Referring to FIG. 20, one of the rotating components of the large gear assembly 560 may include a plurality of ratchet teeth 561 around the circumference of the rotating component, with the ratchet teeth positioned radially outward of the teeth of the large gear 562. In the illustrated example, there are two sets of ratchet teeth 561, with the teeth of the large gear 562 positioned between the ratchet teeth 561. The ratchet teeth 561 may be part of two cam tracks 570 (labeled in FIG. 20) which may be positioned outside of two corresponding crimping guides with radial slots (which may be similar or identical to the crimping guides with radial slots of crimper 300), with the crimping guides being fixed to the housing assembly 510 and positioned on either side of the large gear 562 and the crimping members (which may be identical to the crimping members described in connection with crimper 300). It should be understood that cam tracks 570 may include spiral grooves similar or identical to spiral grooves 372, although those spiral grooves are not illustrated on cam tracks 570 in the figures. The ratchet teeth 561 may include a ramped surface that extends in the anti-clockwise direction (in the view of FIG. 20) such that the height of the ratchet teeth 561 increases in the anti-clockwise direction. At the maximum height of the ramped surface of the ratchet teeth 561, the ratchet teeth 561 may project downward back toward the large gear assembly 560 so as to present a generally flat surface in the anti-clockwise direction in the view of FIG. 20. Referring now to FIG. 21, when the tab 547 is in the up or locked condition shown in FIG. 17, the interference mechanism 620 is positioned relatively close to the large gear assembly 560 so that the ratchet teeth 561 engage with the ratchet teeth 625. In this configuration, the large gear 562 is generally free to be rotated in the clockwise direction in the view of FIG. 21 to cause the iris assembly 580 to close, but the large gear 562 is unable to be rotated in the anti-clockwise direction in the view of FIG. 21 because that flat surfaces of the ratchet teeth 561 will engage the flat surfaces of the ratchet teeth 625, preventing the anti-clockwise direction. However, if the tab 547 is pulled downward (from the position shown in FIG. 17) or otherwise moved to the unlocked condition, the actuator 546 will rotate in the anti-clockwise direction of FIG. 22, pulling with it the interference mechanism 620 away from the large gear assembly 560. As shown in FIG. 22, after the interference mechanism 620 has been rotated about the pins 621 in the anti-clockwise direction, the ratchet teeth 625 are spaced away from the ratchet teeth 561, allowing the large gear 562 to rotate in either the clockwise or anti-clockwise direction to close or open the iris assembly 580.

Although not all separately illustrated in FIGS. 19-22, the large gear assembly 560 may include the same components as described in connection with the large gear assembly 360 of crimper 300. In other words, the large gear assembly 560 may include cam tracks 570 with spiral grooves (not shown in the drawings) and crimping guides with radial slots. Similarly, the iris assembly 580 may be similar or identical to the iris assembly 380 of crimper 300, including the use of a plurality of crimping members with pins that ride along the radial slots of the crimping guide and the spiral grooves of the cam tracks 570. Thus, these components are not described again for crimper 500.

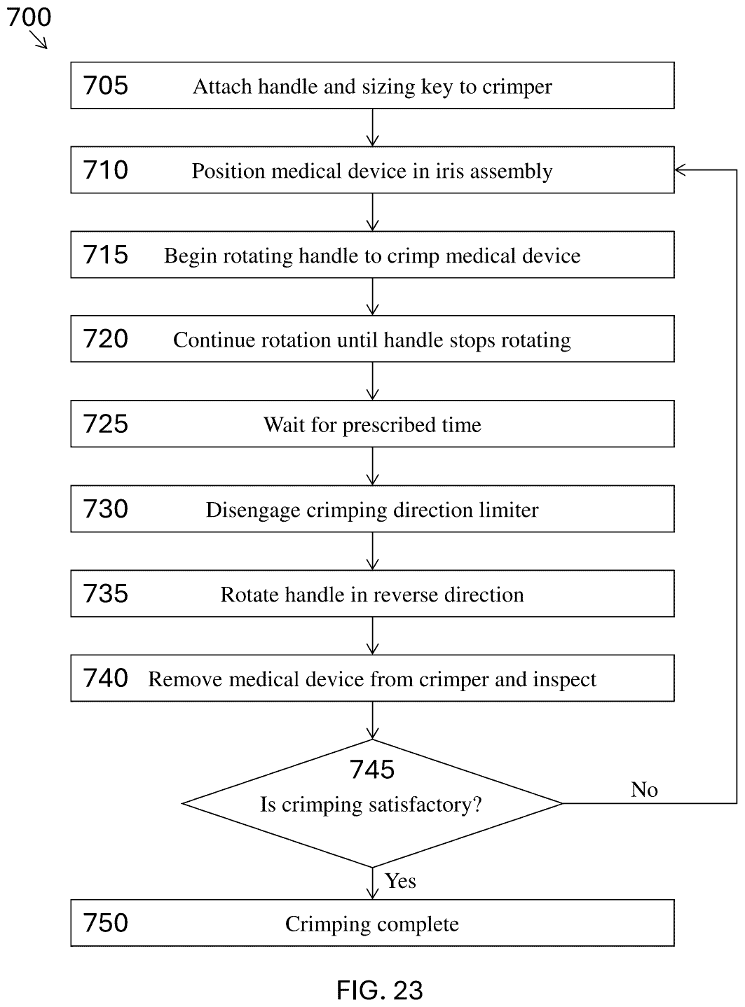

FIG. 23 is a flowchart of example steps of an exemplary method 700 of crimping a medical device according to one aspect of the disclosure. It should be understood that not all steps shown in the flow chart of method 700 are strictly required to be performed, and other steps not listed may also be performed. In a first exemplary step 700, after removing any necessary components from packaging, the handle assembly 520 may be inserted into the central opening 544 of the pinion 542, either on the left size for left-handed crimping or on the right side for right-handed crimping per the user's preference. As explained above, the handle assembly 520 may be limited to connecting to the pinion 542 in a single orientation (in each of the left-handed and right-handed crimping configurations), which may help ensure that the handle assembly 520 is connected to the crimper 500 in the correct orientation. During this initial step 705, the sizing key 600 may also be connected to the crimper 500 by inserting it into the correspondingly shaped opening 610. As explained above, the sizing key 600 may be provided as a component of the medical device and/or delivery system packaging so that the sizing key 600 will limit crimping to the prescribed amount for the particular medical device being used in the crimper 500. The user may also confirm that the sizing key 600 matches the correct sizing for the medical device, for example by reviewing any sizing indicia provided on the sizing key 600 compared to the medical device. It should be understood that the sizing key 600 may be coupled to the crimper 500 either before or after the handle assembly 520 is coupled to the crimper 500. Further, the use of the sizing key 600 may be optional, but may be recommended for all cases.

After the sizing key 600 and the handle assembly 520 are coupled to the crimper 500, the use may position the medical device within the iris assembly 580 while the iris assembly 580 is in the open condition in step 710. The crimper 500 may be supplied to the user with the iris assembly 580 already in the open condition, but if the iris assembly 580 is for any reason not already in the open condition, the user may open the iris assembly 580 in a manner similar to that described in connection with step 730 and step 735 below. If the medical device is a balloon expandable prosthetic heart valve (e.g. prosthetic heart valve 10), it may be positioned over a balloon (e.g. balloon 136) of a delivery system (e.g. delivery system 100), and the prosthetic heart valve may be positioned within the iris assembly 580 manually while it is positioned over the balloon on the catheter of the delivery system.

While the medical device is suitably positioned within the iris assembly 580, the user may grip the handle assembly 520 and begin to rotate the handle assembly 520 in step 715. In step 715, the tab 547 is preferably in the up or locked condition shown in FIG. 17. As the user begins to rotate the handle assembly 520, the handle assembly 520 forces the pinion 542 to rotate, which in turn forces the large gear 562 to rotate, which in turn drives the iris assembly 580 to begin to close in substantially the same fashion described in connection with crimper 300 (e.g. crimping elements are forced radially inwards by rotation of a spiral grooves of a cam track while guided by radial slots of a crimping guide). Because tab 547 is in the up or locked condition, the crimping direction limiter is in an engaged condition so that the ratchet teeth 625 of the interference mechanism 620 engage with the ratchet teeth 561 on the large gear assembly 560. With this configuration, during the initial crimping of step 715, the user may feel and/or hear a “clicking” as the ratchet teeth successively engage, allowing for the iris assembly 580 to reduce in size for crimping, but disallowing the handle assembly 520 from rotating in the wrong direction to open the iris assembly 580.

In step 720, the user may continue to rotate the handle assembly 520 to close the further iris assembly 580 and to further crimp the medical device, until the handle assembly 520 stops completely. The stopping of the handle assembly 520 may be caused by engagement of the stops 563 of the large gear assembly 560 with the sizing key 600 (e.g. the inner or bottom portion 604 of the sizing key 600). This engagement will prevent further rotation of the large gear 562, and thus will prevent further rotation of the pinion 542, causing the handle assembly 520 to encounter high resistance so that further rotation of the handle assembly 520 is prevented. At this point, the medical device may be fully crimped according to the prescribed crimping size, which may also correspond to the specific sizing key 600 used in the procedure. For some medical devices, including balloon-expandable prosthetic heart valves, the medical device may have a tendency to recoil or to otherwise increase in size after a crimping force is removed. To help avoid and/or minimize this recoil, in step 725, the user may take no action (e.g. wait) for a prescribed time, such as 30 seconds, although other times (e.g. as little as 15 seconds or as much as one or two minutes) may be suitable. Because the tab 547 remains in the up or locked condition shown in FIG. 17 during this waiting step 725, the crimping direction limiter remains engaged ensuring that, even if the user removes his grip from the handle assembly 520, the iris assembly 580 will not increase in size due to relaxation or recoil of the medical device, since the ratchet engagement prevents such action.

After waiting the prescribed time in step 725, the user may move tab 547 to the down or unlocked condition in step 730 which, as described in greater detail below, disengages ratchet teeth 625 from ratchet teeth 561 via rotation or movement of the actuator 546 and corresponding rotation or movement of the interference mechanism 620. After the crimping direction limiter is disengaged in step 730, the user may rotate the handle assembly 520 in the reverse direction in step 735 to rotate the pinion gear 542 and the large gear 562 in the reverse direction (which is no longer prevented due to the disengagement of the ratchet teeth) to open the iris assembly 580. During step 735, the user may maintain force on the tab 547 to help ensure that the crimping direction limiter remains disengaged, although in some instances it may not be required to maintain force on the tab 547 during step 735.

After the iris assembly 580 has been re-opened, the user may remove the medical device from the crimper 500 to inspect it in step 740, and optionally may re-engage the crimping direction limiter by moving the tab 547 back to the initial up or engaged position, for example as shown in FIG. 17. If the medical device is a balloon-expandable prosthetic heart valve, the user may determine whether the prosthetic heart valve appears to be appropriately crimped and positioned relative to the balloon of the balloon catheter. Regardless of the specific medical device being used, if the user determines that the medical device needs to be crimped again in step 745, the user may repeat the process beginning at step 710. If the user determines the medical device has been satisfactorily crimped, the user may consider the crimping process complete at step 750, and move forward with any remaining aspects of the medical procedure, such as implantation of a prosthetic heart valve into a patient.

It should be understood that various components of the crimper 500 may be removed or modified while still being able to achieve crimping functionality. For example, one or both of the crimping direction limiter and the sizing key may be omitted, and the crimper 500 may still function appropriately for crimping a medical device, although certain functionality may be lost if one or both of those components were omitted.

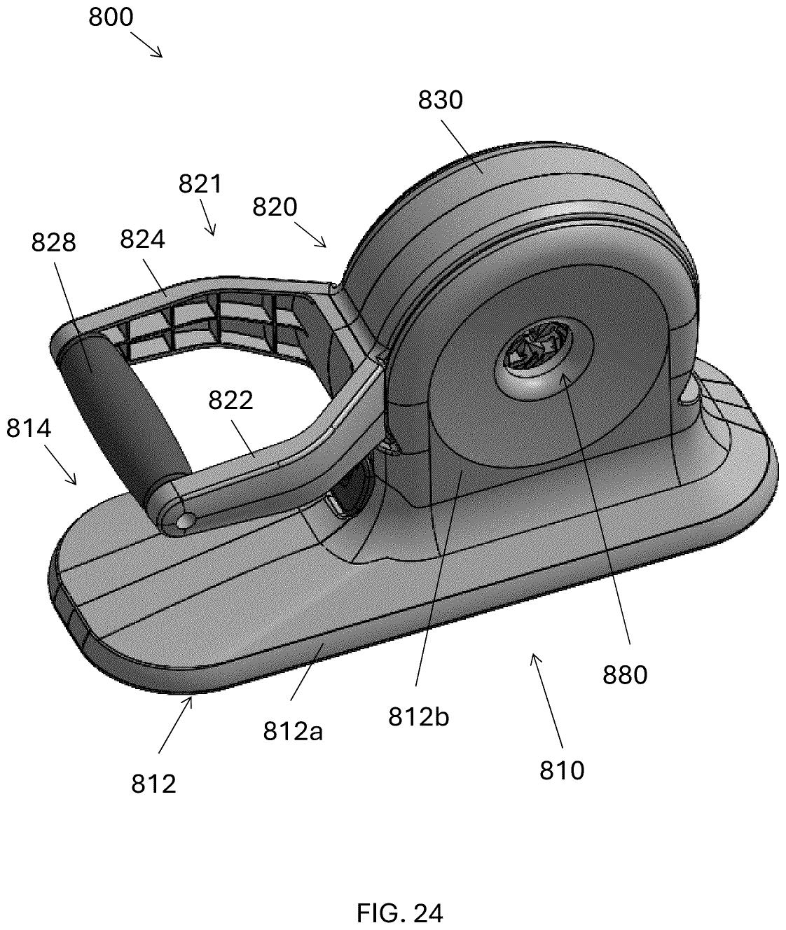

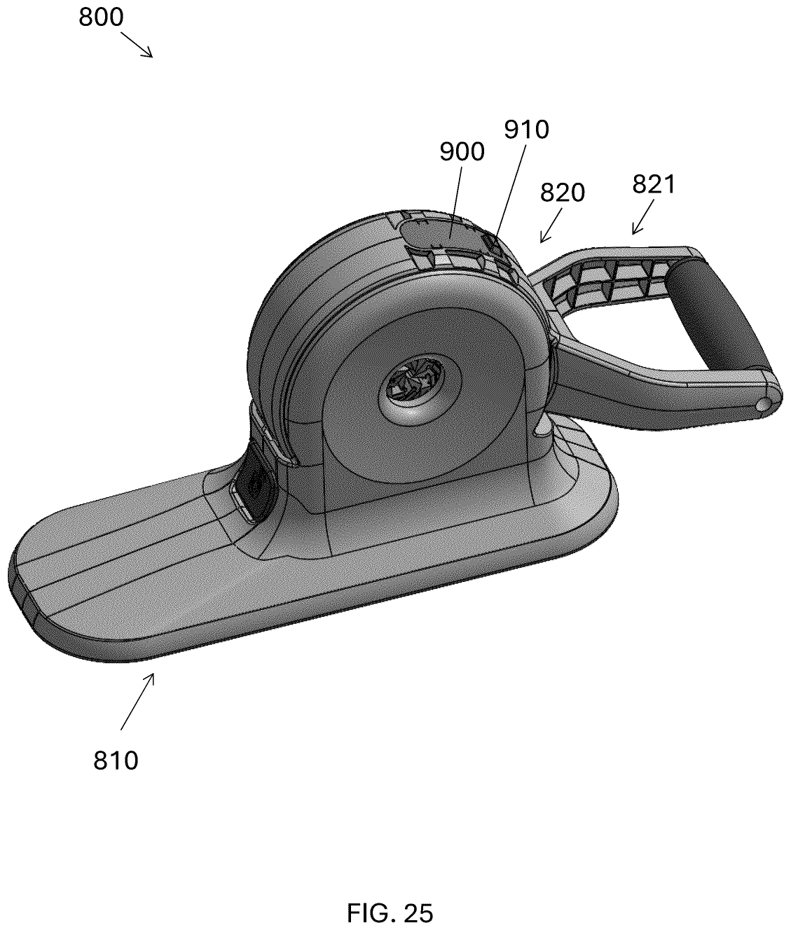

FIGS. 24-25 show perspective views of a crimper 800 according to a further aspect of the disclosure. One of the primary differences between crimper 800 and other crimpers disclosed herein is that crimper 800 does not include a geared drive system in which a handle rotates a pinion gear which in turn rotates a larger gear to drive crimping. Rather, crimper 800 may be thought of as a direct drive crimper in which actuation of a handle directly drives the crimping without the need for intermeshing gears. However, crimper 800 has additional differences compared to other crimpers described herein, which are described in greater detail below.

Referring to FIG. 24, which shows the crimper 800 in a closed condition, the crimper 800 may include a housing assembly 810. In some examples, the housing assembly 810 may be provided as a shell having two side portions that are coupled together. For example, the housing assembly 810 may include a first side member 812 and a second side member 814 that are secured together to form the housing assembly 810. The first side member 812 may include a base portion 812a and a generally circular housing portion 812b, with the opening for receiving the medical device (and through which the crimping members will come together during crimping) being formed at a center area of the circular housing portion 812b. The second side member 814 may be substantially a mirror image of the first side member 812, including a base portion 814a and a generally circular housing portion 814b.

Still referring to FIGS. 24-25, the crimper 800 may also include a crimping assembly 820. The crimping assembly 820 may include a handle assembly 821, a rotating body 830, and other components described in greater detail below. The handle assembly 821 may include two arms 822, 824 each having a first end coupled to the rotating body 830, and opposite second ends coupled to a grip 828. In some examples, the arms 822, 824 may be integrally formed with the rotating body 830, although in other examples the arms 822, 824 may be separately coupled (e.g. via fasteners) to the rotating body 830. In the illustrated example, the rotating body 830 may be provided in two halves 832, 834, and the arms 822, 824 may each be integrally formed with respective ones of the halves 832, 834 of the rotating body 830. The grip 828 is preferably in the general shape of a cylinder, although not necessarily in a perfectly cylindrical shape, and is preferably formed of (or coated or otherwise covered with) a soft material. As is explained in greater detail below, the user may perform crimping by gripping the grip 828 while the crimper is in the open condition (the handle assembly 821 is in the position shown in FIG. 25), and pulling the handle assembly 821 toward the user, thereby rotating the rotating body 830 and closing the crimper 800. Unlike other crimpers disclosed herein, the handle assembly 821 is generally centered along the sides of the crimping assembly 820, such that the force applied by the user to the handle assembly 821 is generally centered along the base of the crimper 800 (e.g. the base portion 812a of the first side member 812 and the base portion 814a of the second side member 814). Thus, there may be little or no risk of the crimper 800 tipping over to the side upon application of a crimping force to the handle assembly 821. However, the base of the housing assembly 810 may extend a distance toward the user so that the user may prefer to hold the base with one hand while activating the crimping assembly 820 using the handle assembly 821 with the other hand. It should be understood that, in FIG. 25, the crimping members are shown in a closed/crimped condition, despite the fact that the crimper 800 in FIG. 25 is actually in the open condition.

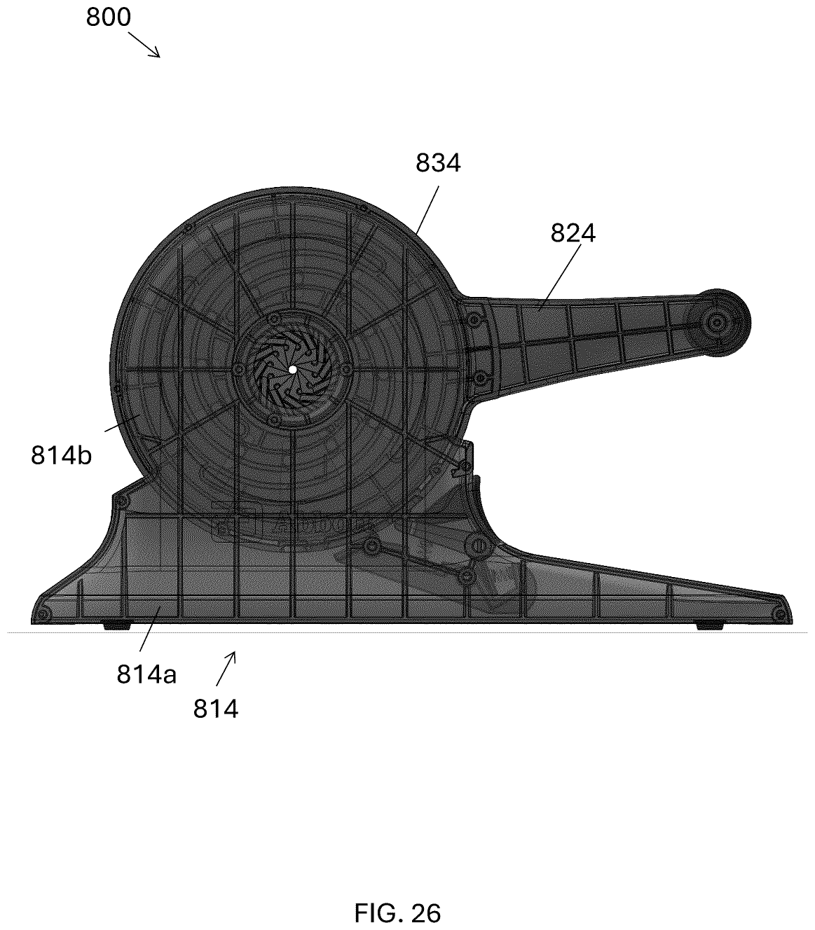

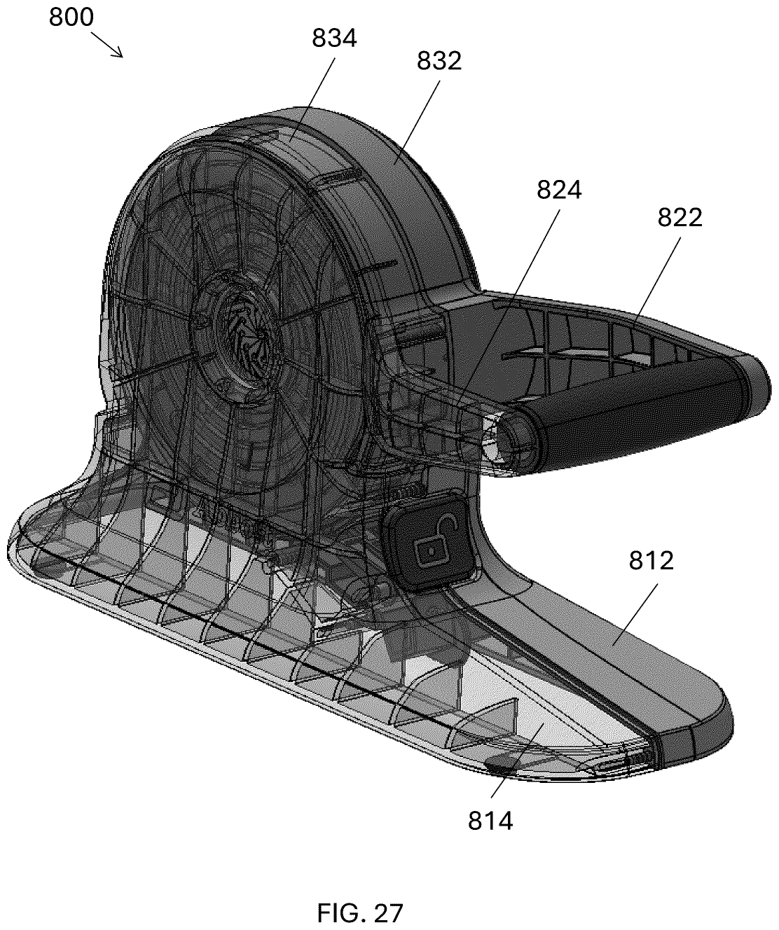

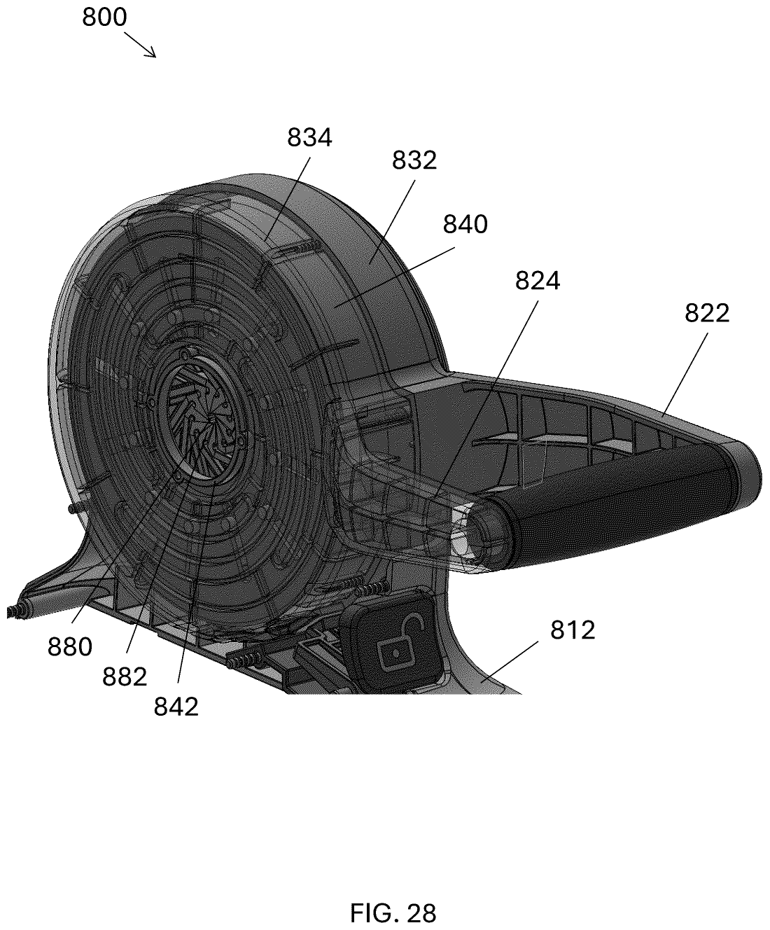

FIGS. 26-27 show side and perspective views, respectively, of the crimper 800 with the second side member 814 of the housing assembly 810 shown in phantom. Similarly, one half 834 of the rotating body 830 is shown in phantom in FIGS. 26-27. FIG. 28 shows a perspective view of the crimper 800 with the second side member 814 of the housing completely omitted from the drawing, with the second half 834 of the rotating body 830 being shown in phantom. Referring generally to FIGS. 26-28, the second half 834 of rotating body 830 may form a cam track with spiral grooves, which may be substantially identical to cam track 370 and spiral grooves 372. Although not visible in the views of FIGS. 26-28, the first half of the rotating body 832 may also form a cam track and spiral grooves that are a mirror image of those formed by the second half 834 of the rotating body 830. The cam tracks are not visible in FIGS. 24-25 because they are positioned just interior to the housing portions 812b, 814b of the side members 812, 814 that form the housing assembly 810. The cam tracks may include a central opening that, in part, defines the iris assembly 880 which receives the medical device. With this configuration, rotation of the handle assembly 821 directly causes corresponding rotation of the cam tracks on the two halves 832, 834 of the rotating body 830. This is why the configuration may be referred to as a “direct drive” compared to the gear-driven crimpers disclosed elsewhere herein.

In addition to the two halves 832, 834 of the rotating body 830 including cam tracks with spiral grooves, as best seen in FIG. 28, crimping guides 840 may be placed interior to the rotating body 830. For example, a pair of crimping guides 840 may be positioned directly adjacent to (and interior of) the cam track of each of the two halves 832, 834 of the rotating body 830. It should be understood that, in FIG. 28, only one of the crimping guides 840 is visible through the phantom half 834 of the rotating body 830. The crimping guides 840 may be substantially similar or identical to the first crimping guides 390 described above, and may for example include a plurality of radial slots. The crimping guides 840 may include a circular rim 842 that is generally coaxial with the circular opening of the cam tracks and which, in part, defines the iris assembly 880. In the illustrated embodiment, the rim 842 may have a smaller diameter than the openings of the cam tracks and may include one or more apertures to receive fasteners. The fasteners may extend through the apertures in the rim 842 (or in apertures adjacent to the rim) and into the interior side of the housing portion 812b (or 814b) to secure the crimping guide 840 to the side member 812 (or 814) of the housing assembly 810. With this configuration, one crimping guide 840 will be coupled to the side member 812, and the other crimping guide 840 will be coupled to the side member 814, with the radial slots of the crimping guides 840 generally aligning with portions of the spiral grooves of the adjacent cam tracks, and with the crimping guides 840 being fixed stationary relative to the housing assembly 810.

Still referring to FIG. 28, the crimper 800 may also include a plurality of crimping members 882, which may be similar or identical to crimping members 382. For example, the crimping members 882 may each include a pair of pins extending outwardly from a first end (or near a first end) of the crimping members 882, with a second end serving as part of a crimping surface. As with crimping members 382, the pins of the crimping members 882 may pass through both the radial slots of the crimping guides 840 and the spiral grooves of the cam tracks of the two halves 832, 834 of the rotating body 830. With this configuration, as in the other crimpers described herein, as the cam tracks rotate (by rotation of the handle assembly 821), the spiral grooves of the cam tracks also rotate, forcing the pins of the crimping members 882 to ride along the spiral grooves and also up or down the radial slots of the crimping guides 840 to either collectively draw the crimping members 882 closer together to close the iris assembly 880 or to draw the crimping members 882 apart from each other to open the iris assembly 880, in substantially the same fashion as described for other crimpers herein. Thus, for the extent of the crimper 800 that has been described to this point, the major difference is the fact that the cam tracks are direct driven by a centrally-positioned handle assembly 821, instead of being driven by a gear assembly via a handle located on only one side of the crimper. However, crimper 800 also includes additional features described in greater detail below.

Referring briefly back to FIG. 25, the crimper 800 may also include a sizing key 900. The sizing key 900 may have a generally similar purpose as sizing key 600, although not identical. As shown in FIG. 25, the sizing key 900 may have a shape that generally follows the contours of the circumferentially outer surface of the rotating body 830, and be configured to snap or otherwise fit into the circumferentially outer surface of the rotating body 830. As with sizing key 600, sizing key 900 may be provided with the medical device and/or the delivery system thereof and be keyed to the specific minimum crimping size desired for the particular medical device being used in the procedure. Unlike sizing key 600, sizing key 900 may have a protrusion 910 extending radially outward from the sizing key 900 when the sizing key 900 is coupled to the rotating body 830. In the view of FIG. 25, the farther to the right that the protrusion 910 is positioned on sizing key 900, the smaller the minimum crimp will be, while the farther to the left that the protrusion is positioned on sizing key 900, the larger the minimum crimp will be. Also unlike sizing key 600, sizing key 900 may rotate and travel along with the rotating body 830, whereas sizing key 600 is fixed to the housing assembly 510 of crimper 500. Still further, unlike sizing key 600, sizing key 900 may provide for not only a device-specific crimping size target, but it may also serve as a holding stop to maintain the crimper 800 at the minimum target size.

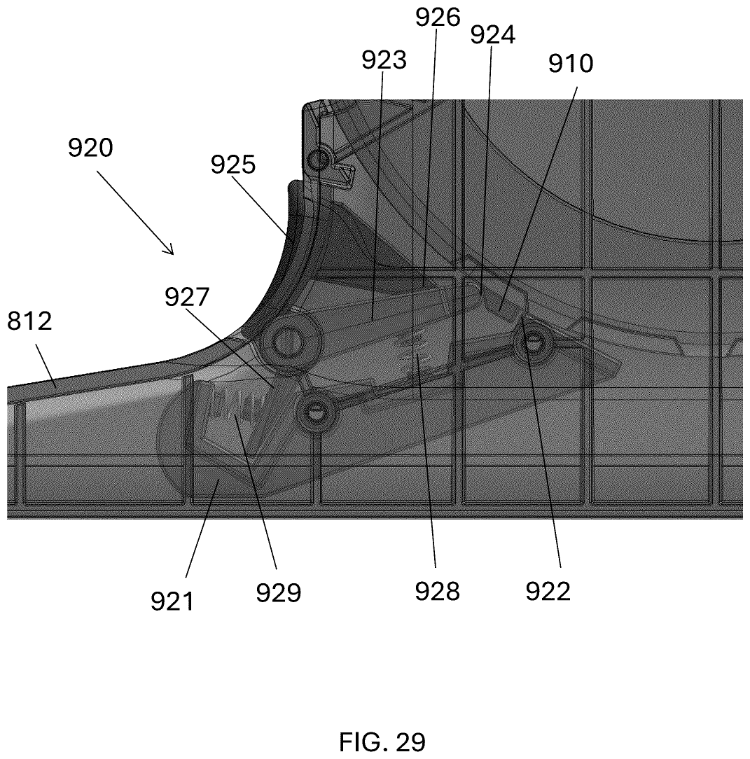

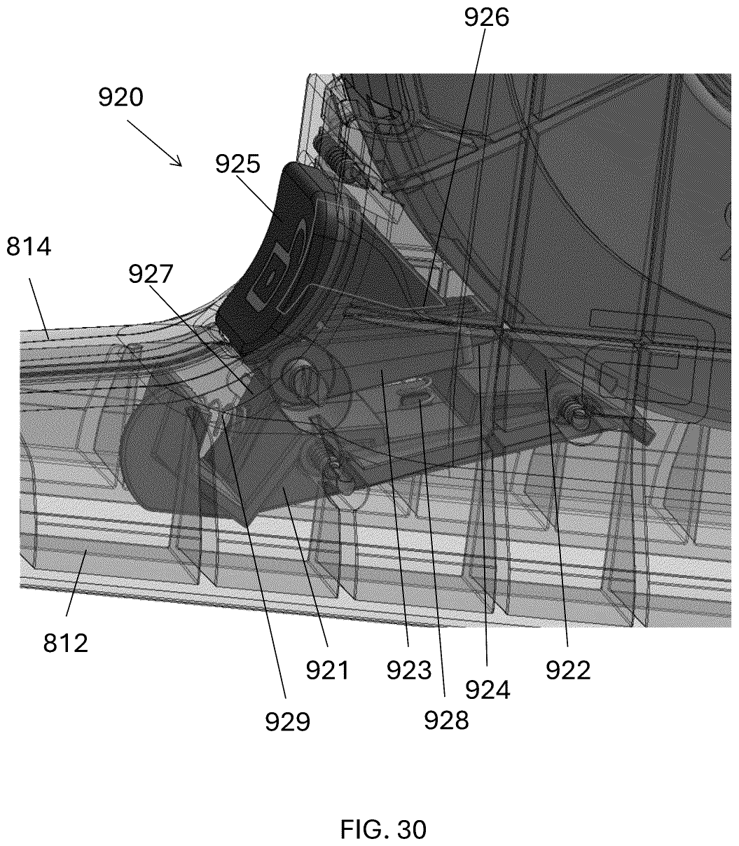

FIGS. 29-30 show enlarged side and perspective views of the crimper with the side members 812, 814 in phantom to better illustrate components of the holding mechanism 920. The holding mechanism 920 may function to interact with the protrusion 910 of the sizing key 900 in order to (i) ensure the iris assembly 880 does not go beyond a minimum size for crimping prescribed for the medical device based on the sizing key 900 and (ii) hold the iris assembly 880 at the prescribed size in the absence of applied forces until the user is ready to actively open the iris assembly 880 to remove the medical device. While FIG. 25 shows the crimper 800 in the open condition (in which the iris assembly 880 is mostly or fully open) with the sizing key 900 near the top of the crimper 800, FIG. 29 shows the crimper 800 after the iris assembly 880 has been reduced to the minimum size permitted by the sizing key 900, with the sizing key 900 having rotated with the rotating body 830 so that the sizing key 900 is near the bottom of the crimper 800.

As shown in FIGS. 29-30, the holding mechanism 920 may include a fixed member 921 that is secured to the housing assembly 810 so that the fixed member 921 is unable to move with respect to the housing assembly 810 (for example via two or more fasteners). One end of the fixed member 921 may present a stop surface 922 which interfaces with the protrusion 910 of the sizing key 900 to stop the rotating body 830 from rotating beyond the prescribed limit for the medical device. In other words, as the medical device is crimped by rotating the handle assembly 821, the handle assembly 821 will be forced to stop rotating once the protrusion 910 contacts the stop surface 922 of the fixed member 921. As the crimper 800 nears the end of its allowable rotation, prior to contacting the stop surface 922, the protrusion 910 will engage a holding surface 924 of a pivoting member 923. A spring 928 that is positioned between the fixed member 921 and the pivoting member 923 may compress upon this contact to allow the protrusion 910 to pass by the pivoting member 923 as the pivoting member 923 pivots. However, after the protrusion 910 clears the holding surface 924 of the pivoting member 923, the spring 928 will decompress to allow the pivoting member 923 to pivot back to its original condition.

The holding mechanism 920 may also include a release button 925 which is positioned within an opening of the housing assembly 810 so that the release button 925 is accessible to be pressed by the user. The release button 925 may include a first extension 926 with a contact surface that contacts a surface of the pivoting member 923 opposite the surface that engages the spring 928. The release button 925 may also include a second extension 927 that engages a surface of the fixed member 921, with another spring 929 being positioned in contact with both the second extension 927 and the fixed member 921. The second extension 927 may be pivotably coupled to the housing assembly 810. In some examples, the pivoting member 923 and the second extension 927 share a pivot axis. After the protrusion 910 clears the holding surface 924 and engages the stop surface 922 so that the pivoting member 923 pivots back to its original position, the holding surface 924 prevents the protrusion 910 from further rotation in a direction away from the stop surface 922. In other words, in the condition shown in FIG. 29, if the user tried rotate the handle assembly 910 to open the iris, the protrusion 910 would apply a generally upward force to the pivoting member 923 at the holding surface 924, which would be transferred to the release button 925 via the first extension 926, causing the release button to attempt to pivot about the pivot axis on the second extension 927. However, because the second extension 927 already is pressing against the fixed member 921, and the fixed member 921 cannot move, the protrusion 910 is not able to rotate upon trying to open the iris assembly 880 by pushing the handle assembly 910. Thus, while crimper 600 included a crimping direction limiter which provided a ratcheting function to create only unidirectional rotation of the crimper 600 as the iris assembly 680 closes, the crimper 800 prevents opening of the iris assembly 880 only when the iris assembly 880 has been reduced to its minimum prescribed size based on the particular sizing key 900 assembled to the crimper 800. If the user desires to open the iris assembly 880, the user may press the release button 925 inwards, which compresses spring 928 and forces the pivot member 923 so that the holding surface 924 clears away from the protrusion 910, allowing the rotating body 830 to again rotate in the direction to open the iris assembly 880. It should be understood that the optional second spring 929 is provided to help ensure that the release button 925 does not move or “wiggle” when the protrusion 910 engages the pivoting member 923 and compresses the spring 928 as the protrusion 910 advances toward the stop surface 922 of the fixed member 921. With this second optional spring 928, the release button 925 will remain stable and in the same position at any time that it is not being intentionally depressed by a user.

Crimper 800 may be used according to a method that is substantially similar or identical to method 700 shown and described in connection with FIG. 23. However, step 730 of method 700 may instead be replaced by a step in which the release button 925 is depressed to allow the user to move to step 735 and rotate the handle assembly 821 in a reverse direction to open the iris assembly 880. Similarly, step 705 may be omitted since the handle assembly 821 is already assembled to the crimper 800.

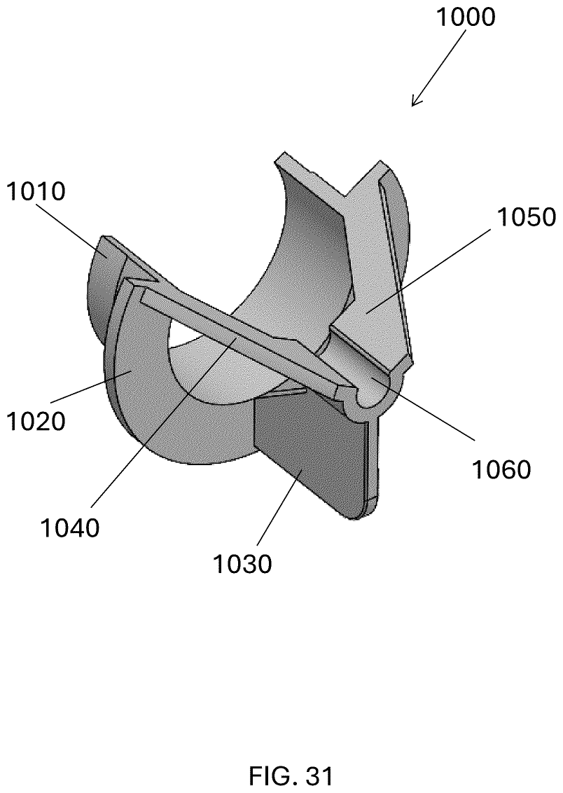

FIG. 31 is a perspective view of a retainer 1000 that may be used with any of the crimpers described herein. Retainer 1000 may be an accessory that can be assembled to the crimper (e.g. the iris assembly) and which may secure either a portion of the medical device, or an accessory such as a sheath of a delivery catheter, to assist with both positioning and maintain the medical device within the iris assembly of the crimper during the crimping process. In the illustrated embodiment, the retainer 1000 includes an insert 1010 which may form a portion of a cylinder and be sized to be inserted into the crimper adjacent the iris assembly (e.g. into the opening of the generally circular housing portion 812b). The insert 1010 is shown as being open on one end, which may assist in coupling a portion of the medical device (or an accessory thereof) to the retainer, although in other embodiments the insert 1010 may by a complete circle or cylinder. A rim 1020 may extend radially outward from a proximal end of the insert 1010, with the rim 1020 having a similar partial circular shape (or a full circular shape if the insert 1010 is fully circular/cylindrical). When the retainer 1000 is assembled to the crimper, the rim 1020 may help ensure the retainer 1010 is inserted to the desired depth in which the rim 1020 contacts or abuts the housing assembly of the crimper. A grip 1030 may extend proximally from the rim, and may have a flat shape generally intended to be gripped by a user (e.g. using the thumb and index finger). Two support arms 1040, 1050 may extend from the proximal end of the rim 1020 and converge to an area that is generally coaxial with the center longitudinal axis of the insert 1010. The two support arms 1040, 1050 may couple to a tip of the grip 1030, and collectively form a groove 1060, which may be generally semi-circular and be sized to accept a particular portion of a medical device or an accessory thereof. It should be understood that the specific design of the retainer 1000 may be different than described, although preferably the retainer 1000 has the minimum capability to attach to the crimper and to support the medical device (via a portion of the medical device or via an accessory) so that the medical device can be generally centered within the iris assembly and maintained in that position by the retainer 1000.

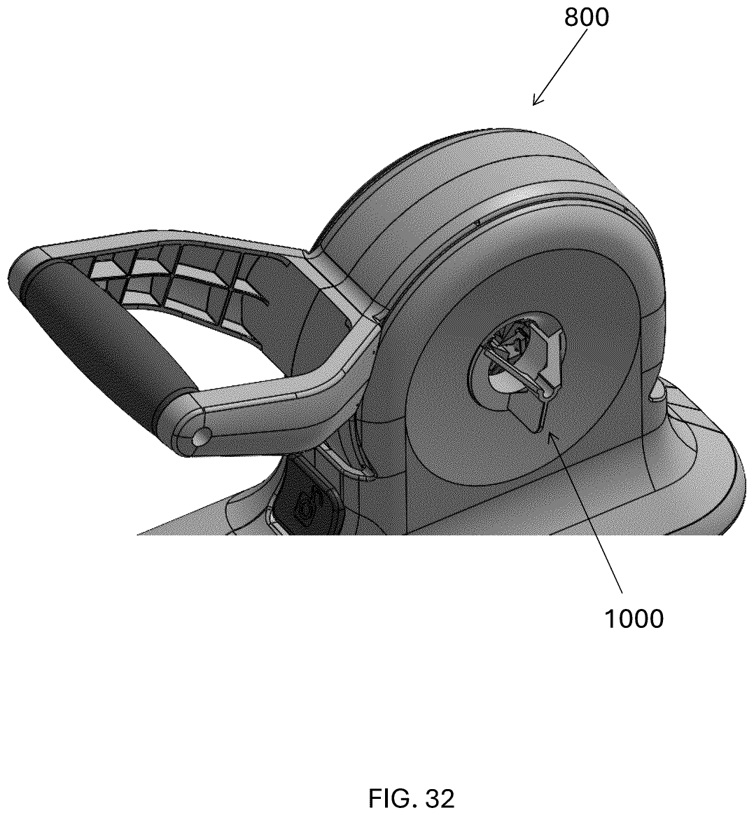

FIG. 32 is a perspective view of the retainer 1000 having been assembled to crimper 800. If the crimper 800 and retainer 1000 are intended for use with prosthetic heart valve 10 (or a similar device), the groove 1060 may be sized and shaped to snugly receive a distal portion of outer catheter 132 or steering catheter 135 (e.g. via a snap fit), so that when the catheter is snapped into the groove 1060 of the retainer 1000, the prosthetic heart valve 10 (which may be positioned between the proximal pillow 136a and the distal pillow 136b) is centered within the iris assembly 880, including radial and/or axial centering. With this configuration, crimping may generally be performed according to method 700 (or other similar methods), except that step 710 may include attaching the retainer 1000 to the crimper and the catheter to the retainer 1000 to position the medical device in the iris assembly. The retainer 1000 may be retained in the assembled condition to the crimper in with any suitable mechanism, including for example a snap fit, a friction fit, a taper fit, etc. which may or may not include one or more protrusions (or holes) on the insert 1010 with mate with one or more holes (or protrusions) within or adjacent to the iris assembly of the crimper.

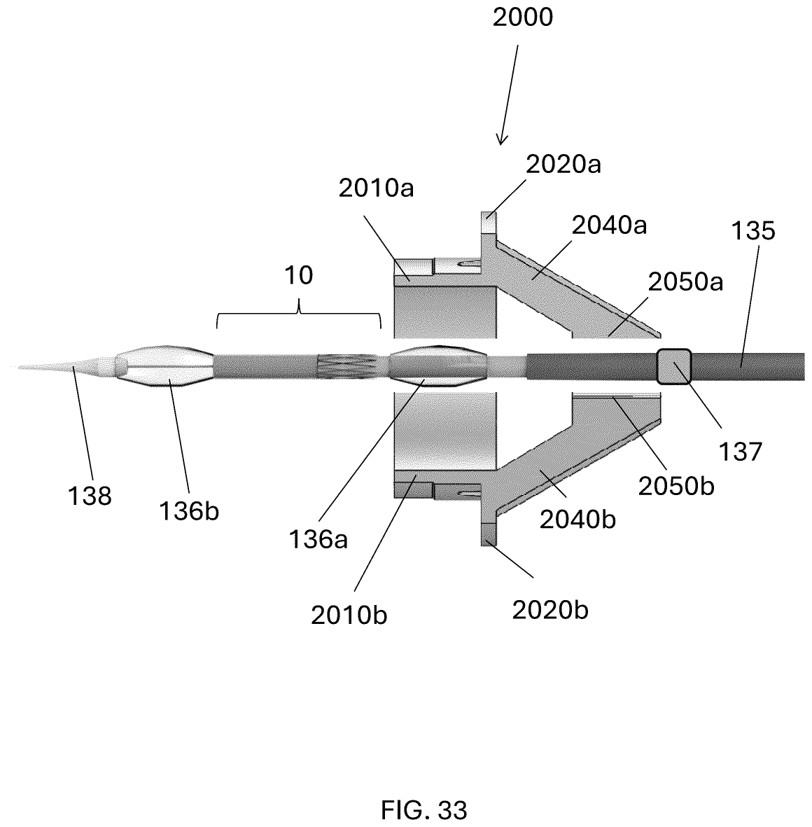

As noted above, the retainer 1000 may have various other specific configurations to achieve the same objective of securing the medical device in the desired position within the iris assembly for crimping. One such alternate example is shown in connection with FIGS. 33-37, which illustrates a retainer 2000 that is formed of two members that clamp over a portion of the medical device (or over an accessory thereof).

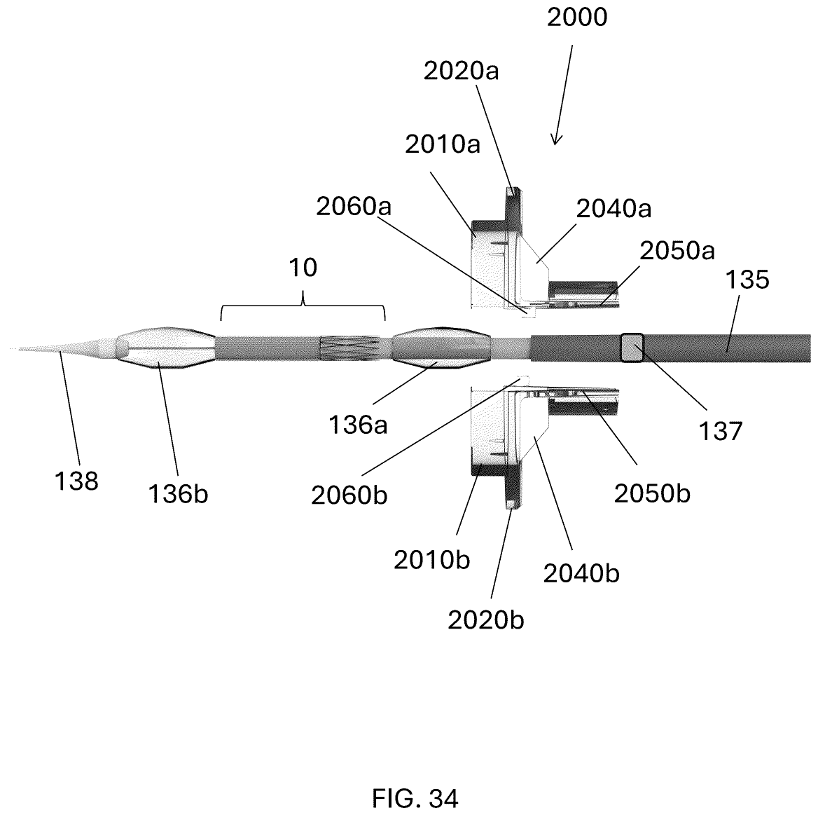

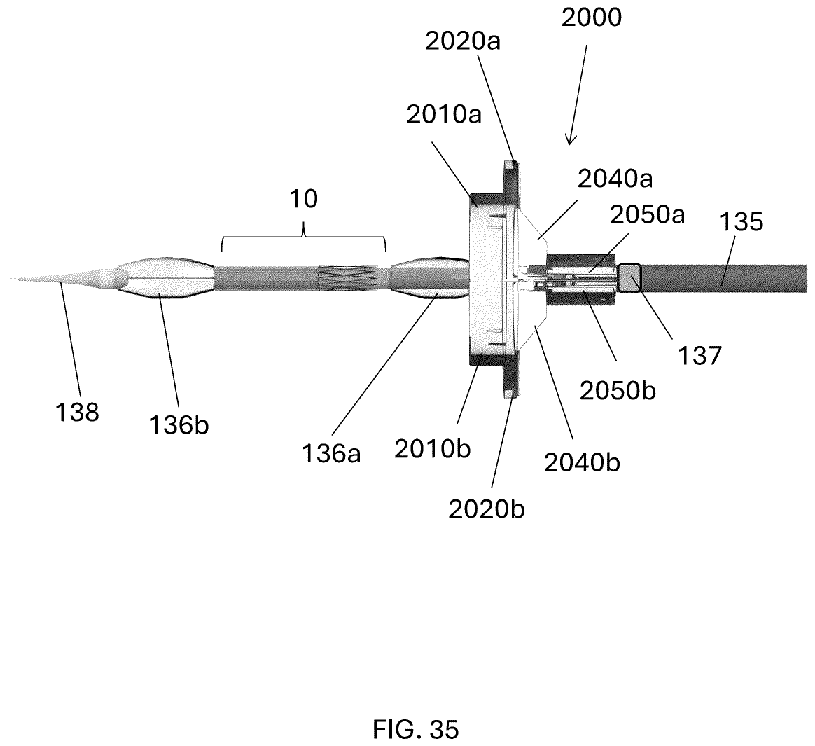

If the medical device is a prosthetic heart valve (such as prosthetic heart valve 10) that is carried on a balloon catheter, the retainer 2000 may secure a portion of the catheter during crimping. FIG. 33 shows a cross-section of the retainer 2000 assembled (e.g. clamped onto or over) steering catheter 135, with the prosthetic heart valve 10 maintained between the proximal pillow 136a and the distal pillow 136b of the balloon 136 (e.g. after having been crimped). FIG. 34 is a side view of the retainer 2000 in position after having been disassembled or unclamped from the steering catheter 135, and FIG. 35 is a side view of the retainer after being disassembled or unclamped from the steering catheter 135. The retainer 2000 may include a first half with a partial insert 2010a and a partial rim 2020a, and a second half with a partial insert 2010b and a partial rim 2020b, which may serve similar purposes to their corresponding components in retainer 1000, when the two halves are assembled or clamped together. The first half may include a support arm or support surface 2040a extending radially inward to a grooved extension 2050a, and the second half may similarly include a support arm or support surface 2040b extending radially inward to a grooved extension 2050b. As best shown in FIG. 34, the first half may include a tab 2060a and the second half may include a tab 2060b, with each tab 2060a, 2060, configured to be received within an opening in the opposite half to secure the two halves together. In the illustrated example, each half of the retainer 2000 is identical to the other half. When the two halves are assembled or clamped together, as shown in FIGS. 33 and 35, the two grooved extensions 2050a, 2050b may form a generally cylindrical recess sized and shaped to securely and snugly fit over an end portion of the steering catheter 135 to secure the steering catheter 135 along the longitudinal center of the assembled retainer 2000. In some examples, the steering catheter 135 may be provided with a marker 137 or any other indicia that identifies a location on the steering catheter 135 to position just outside of the retainer 137. The marker 137 may be a printed line or arrow or any other symbol.

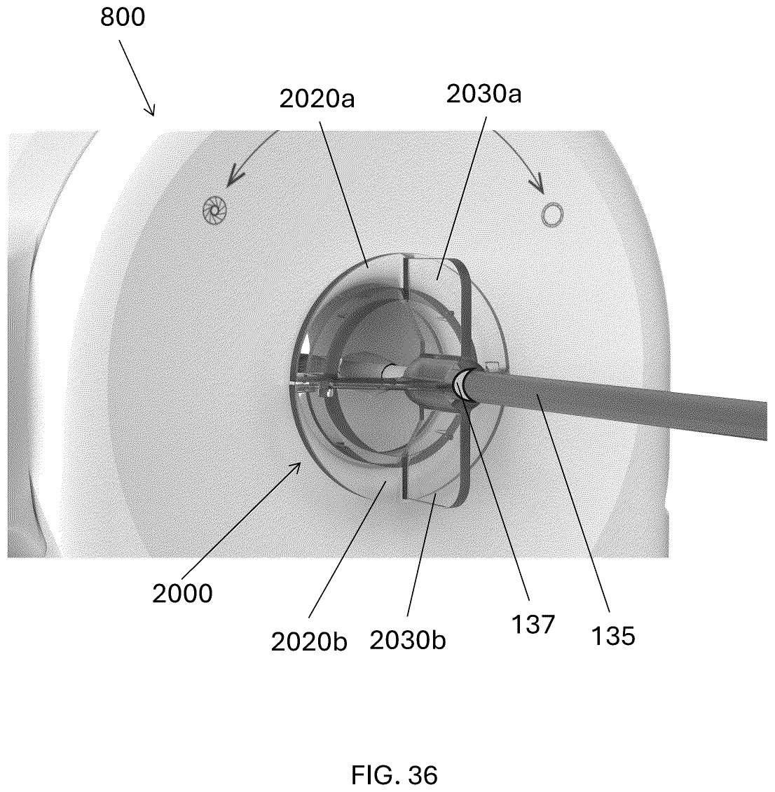

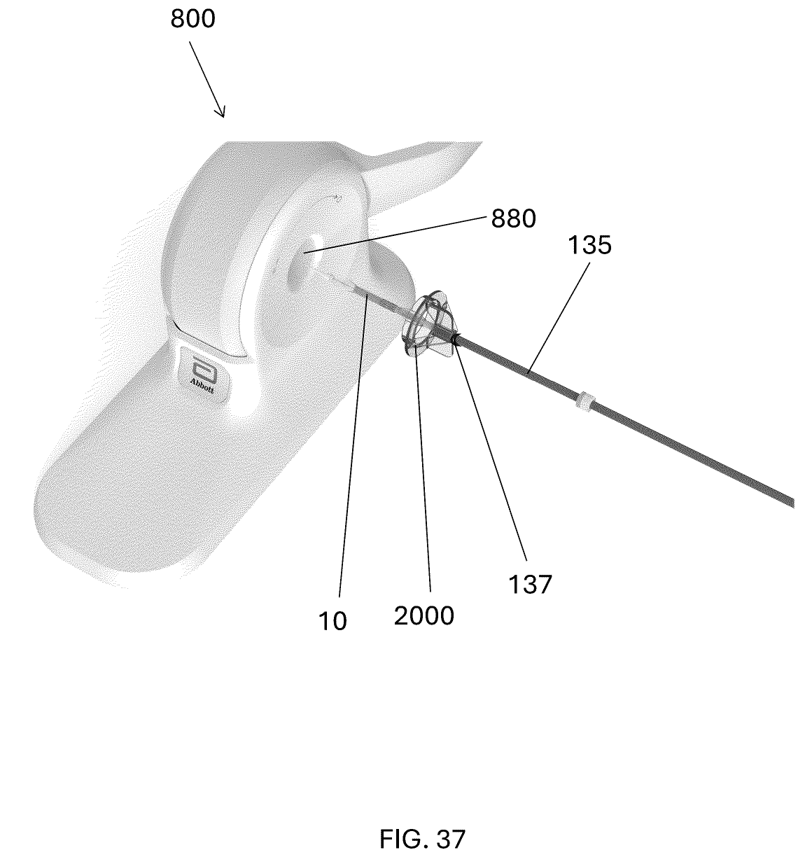

FIG. 37 shows the two halves of the retainer 2000 having been assembled together to each other and over the steering catheter 135, with the alignment marker 137 positioned just proximal to the pair of grooved extensions 2050a, 2050b, after the prosthetic heart valve 10 has been crimped onto the balloon of the delivery device and after being disconnected from the crimper. However, the configuration illustrated in FIG. 37 may also represent the retainer 2000 prior to being coupled to the crimper (although the prosthetic heart valve 10 would be in an expanded condition prior to being initially inserted into the crimper for crimping). FIG. 36 shows the retainer 2000 assembled (e.g. clamped) together over the steering catheter 135, with the alignment marker 137 positioned just proximal to the grooved extensions of the retainer 2000. As with retainer 1000, retainer 2000 may have any suitable connection mechanism to the crimper (and/or the iris assembly thereof), such as a snap fit, an interference fit, a taper fit, etc. which may include tabs or prongs received in corresponding holes or detents. FIG. 36 shows that, in some examples, the two halves of the retainer 2000 may include grips 2030a, 2030b generally similar to the corresponding grip 1030 of retainer 1000. It should be understood that the alignment marker 137 may be positioned such that, when the alignment marker 137 is in the desired position just proximal to the retainer 2000, and the retainer 2000 is assembled to the crimper, the prosthetic heart valve 10 is both axially and radially centered within the iris assembly for crimping. In some examples, retainer 1000 and/or 2000 may be formed of a clear or otherwise see-through material so that the user can visualize the medical device after being crimped without needing to remove the retainer.

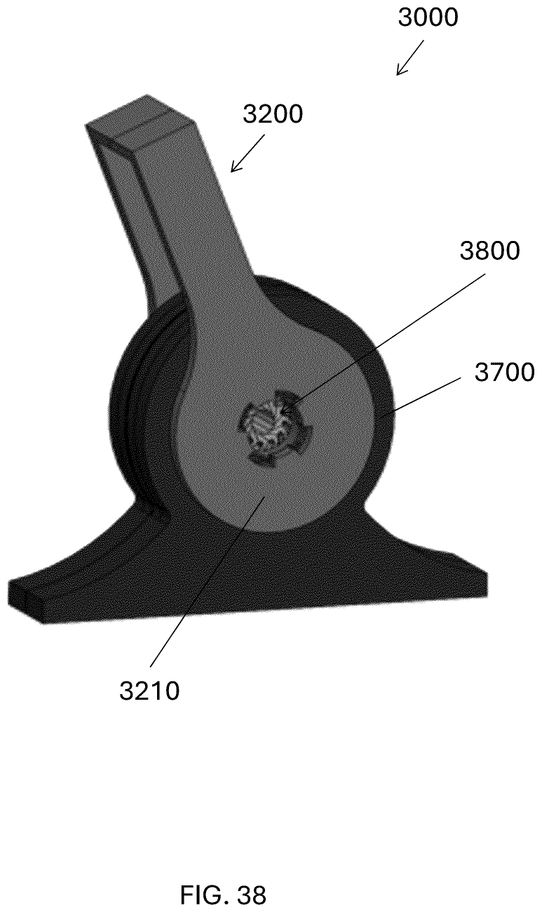

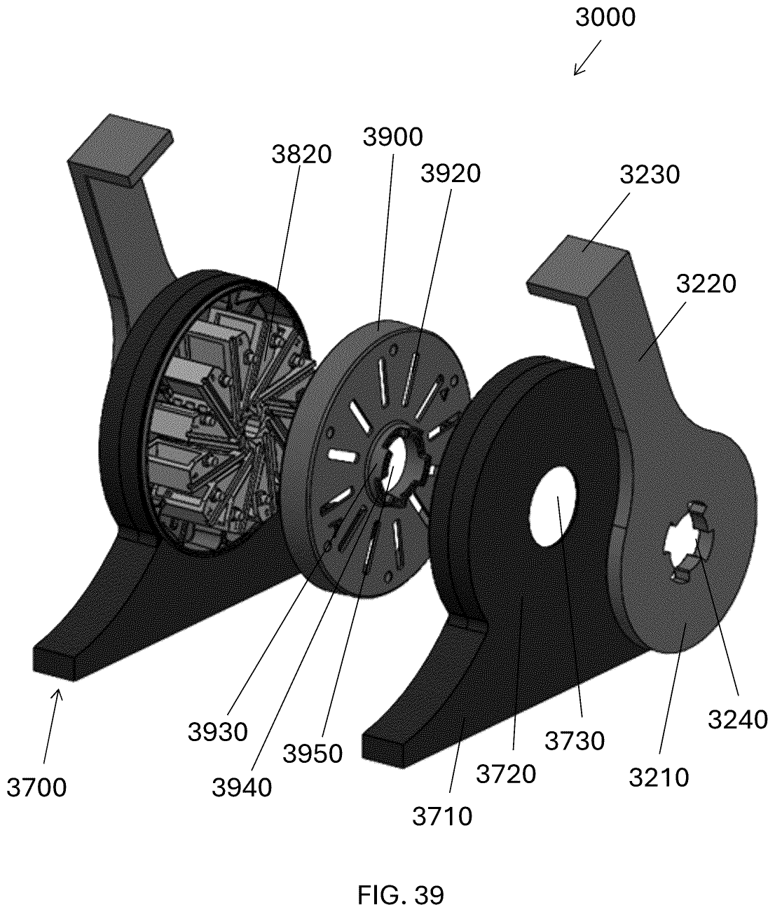

FIGS. 38-39 show another example of a crimper 3000 according to an embodiment of the disclosure. Crimper 3000 may have design elements in common with other crimpers described herein, with one of the main differences being that the iris assembly 3800 undergoes rotation as the iris assembly 3800 opens and closes. As shown in FIG. 39, the iris assembly 3800 may include a plurality of crimping members 3820, which may be similar or identical to other crimping members described herein, and are thus not described in further detail again. The crimping members 3820 and iris assembly 3800 may form the most interior portion of the crimper 3800. The crimping members 3820 may be enclosed within a pair of crimping guides 3900. The crimping guides 3900 may be mostly similar to the other crimping guides described herein, including having a plurality of radial slots 3920 that receive pins or other protrusions of the crimping members 3820 therethrough to guide movement of the crimping members 3820 as the iris assembly 3800 opens or closes. However, one difference between the crimping guides 3900 and the other crimping guides described herein is that the crimping guides 3900 are configured to rotate during crimping. As is described in greater detail below, the crimping guides 3900 may include a rim that outlines a central opening 3950 that receives the medical device, with the rim having alternating protrusions 3930 and recesses 3940 to engage an actuator for rotation.

As shown in FIGS. 38-39, the crimper 3000 may also include a pair of cam tracks 3700 that enclose the crimping guides 3900. Although not shown in FIGS. 38-39, the cam tracks 3700 may include spiral grooves that are similar or identical to the spiral grooves of the other cam tracks disclosed herein. As with the other cam tracks disclosed herein, the spiral grooves of the cam tracks 3700 may also receive pins or other protrusions of the crimping members 3820 that also pass through radial slots 3920. Unlike other cam tracks described herein, cam tracks 3700 may form part of the housing assembly and may be configured to remain stationary during crimping. For example, the two cam tracks 3700 may each include a base 3710, and the bases 3710 may be configured to sit on a table and/or to be clamped or otherwise fixed to a table to ensure that the cam tracks 3700 remain stable during crimping. However, a user may instead manually hold the bases 3710 of the cam tracks 3700 during crimping to maintain stability. A generally circular or annular portion 3720 may extend above the bases 3710, with the spiral grooves being formed in the circular or annular portion 3720. An opening 3730 may be formed in the circular or annular portions 3720 to receive the medical device therethrough to allow for crimping. Further, as is described in greater detail below, the rim of the crimping guides 3900 and/or the protrusions 3930 and recesses 3940 thereof may align with the opening 3730 and at least partially extend through the opening 3730 when the crimper 3000 is assembled.

The crimper 3000 may also include a handle assembly 3200. The handle assembly 3200 may include two actuator portions 3210 which may be generally circular. An arm 3220 may extend from each actuator portion 3210, and the two arms 3220 may be operably coupled to each other. In one example, the two arms 3220 may each include an extension 3230 extending toward the other arm, with the two extensions 3230 being coupled together. In some examples, the extensions 3230 may be integrally formed with each other so that the handle assembly 3200 is a single monolithic unit. In some examples, the extensions 3230 may be connected to each other by any suitable mechanism, such as adhesives, welding, snap fits, mechanical interlocks, etc. In some examples, the two extensions 3230 may be formed as a single member that connects the two arms 3220, for example similar to how grip 828 connects arms 822, 824. With any of these configurations, the handle assembly 3200 has a generally similar configuration to the handle of crimper 800, with the forces being generally centered along the crimper 3000 during crimping.

The actuator portions 3210 may each include a central opening 3240 that, when the crimper 3000 is assembled, aligns with the openings 3730 of the cam tracks 3700 and the openings 3950 of the crimping guides 3900. A plurality of recesses may be formed along the circumference of the opening 3240 which may have sizes, shapes, and configurations that are complementary to the protrusions 3930 of the crimping guides 3900. With this configuration, when the crimper 3000 is assembled, the protrusions 3930 pass through the openings 3730 of the cam tracks 3700 and into the recesses of the opening 3240. The engagement of the protrusions 3930 with the portions of the actuator portions 3210 that form the recesses in the opening 3240 allows for torque transmission between the actuator portions 3210 and the crimping guides 3900. In use, rotating the handle assembly 3200 causes the actuator portions 3210 to rotate about the central axis of the iris assembly 3800, which causes the crimping guides 3900 to correspondingly rotate about the central axis of the iris assembly 3800, while the cam tracks 3700 remain stationary relative to the table or other surface on which they are positioned (which may include being fixedly positioned). As the crimping guides 3900 rotate, the radial slots 3920 correspondingly rotate, forcing the crimping members 3820 to correspondingly rotate due to the pins (or other protrusions) of the crimping members 3820 being received within those radial slots 3920. As in other embodiments described herein, as the pins of the crimping members 3820 rotate relative to the cam tracks 3700 (although in other embodiments it is the cam tracks rotating relative to the crimping members, not vice versa as with crimper 3000), the pins ride along the spiral grooves of the cam tracks 3700 forcing the crimping members 3820 to either collectively move closer to one another to reduce the size of the iris assembly 3800, or away from each other to increase the size of the iris assembly 3800. Thus, as the crimping guides 3900 rotate, the crimping members 3820 also rotate while the pins of the crimping members 3820 slide up or down the radial slots 3920 that at least partially confine movement of the crimping members 3820.

Whereas other embodiments of the crimper described herein allow for the iris assembly to increase or decrease in size without relative rotation between the crimping members and the medical device being crimped by the crimping members, crimper 3000 forces that crimping members 3820 to rotate as they close to reduce the size of the iris assembly 3800 to crimp the device positioned within the iris assembly 3800. This simultaneous rotation of the crimping members 3820 during crimping may provide one or more benefits. For example, if the medical device being crimped is a prosthetic heart valve, such as prosthetic heart valve 10, the simultaneous rotation of the crimping members 3820 with crimping (as the handle assembly 3200 is pulled or pushed by the user) may force the tissue (e.g. the prosthetic leaflets 90 within the frame 20) to spiral. In other words, the prosthetic heart valve 10 may be positioned on the balloon 136 of the delivery system 100 within the iris assembly 3800 during crimping, and the rotation of the crimping members 3820 during crimping may force the prosthetic heart valve 10 to rotate around the balloon 136 as the prosthetic heart valve 10 is crimped. This rotation of the prosthetic heart valve 10 during crimping may result in friction between the prosthetic leaflets 90 and the balloon 136 (which does not rotate), causing the prosthetic leaflets 90 to spiral in the direction opposite the rotation, which may in turn result in a more uniform and predictable positioning of the prosthetic leaflets 90 when the prosthetic heart valve 10 is crimped. The higher uniformity and predictability of how the prosthetic leaflets 90 fold or otherwise collapse during crimping of the prosthetic heart valve 10 may, in turn, allow the prosthetic heart valve 10 to be crimped to a smaller diameter and/or to reduce the likelihood of the prosthetic leaflets 90 to become damaged during crimping. In some examples, the balloon 136 includes pleats or other folds that are wrapped in a rotational direction during crimping of the prosthetic heart valve 10, and the delivery system 100 is positioned during crimping so that the crimping members 3820 rotate during crimping in a direction that matches the direction of the folds or pleats of the balloon 136. This relative orientation may help ensure that the pleats of the balloon 136 are not forced to unfold during crimping of the prosthetic heart valve 10 onto the balloon 136. However, this is just one example and such matching of the rotation of the crimping members 3820 to the directionality of folding of pleats of the balloon 136 is not necessary. It should be understood that any of the holders described above may be used crimper 3000.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A crimping device for crimping a medical device from a first larger diameter to a second smaller diameter, the crimping device comprising:
a housing assembly;
an iris assembly including a plurality of crimping members;
a handle assembly operably coupled to the housing assembly;
a rotating body operably coupled to the handle and to the plurality of crimping members such that rotation of the handle causes rotation of the rotating body, and rotation of the rotating body drives the crimping members toward or away from each other to close or open the iris assembly, respectively; and
a sizing key configured to be coupled to the rotating body,
wherein (i) when the sizing key is not coupled to the rotating body, the rotating body has a first range of rotation corresponding to a first minimum size of the iris assembly, and (ii) when the sizing key is coupled to the rotating body, the rotating body has a second range of rotation corresponding a second minimum size of the iris assembly, the second minimum size of the iris assembly being larger than the first minimum size of the iris assembly.

2. The crimping device of claim 1, wherein the sizing key includes a protrusion that extends radially outward from the rotating body when the sizing key is coupled to the rotating body.

3. The crimping device of claim 2, further comprising a holding mechanism, the holding mechanism including a fixed member secured to the housing assembly, the fixed member defining a stop surface, whereby during rotation of the handle and rotation of the rotating body, while the sizing key is coupled to the rotating body, the protrusion of the sizing key is configured to abut the stop surface of the fixed member when the iris assembly has the second minimum size to limit the iris assembly from a reduction in size beyond the second minimum size.

4. The crimping device of claim 3, wherein the holding mechanism includes a pivoting member defining a holding surface so that, while the sizing key is coupled to the rotating body, the protrusion of the sizing key is configured to abut the holding surface of the pivoting member when the iris assembly has the second minimum size such that the protrusion of the sizing key is positioned between the stop surface and the holding surface.

5. The crimping device of claim 4, wherein while the sizing key is coupled to the rotating body, and while the protrusion of the sizing key is positioned between the stop surface and the holding surface, the rotating body is prevented from rotating to maintain the iris assembly at the second minimum size.

6. The crimping device of claim 5, wherein the holding mechanism includes a release button accessible through the housing assembly, whereby while the sizing key is coupled to the rotating body, and while the protrusion of the sizing key is positioned between the stop surface and the holding surface, depressing the release button moves the pivoting member so that the holding surface is no longer in abutment with the protrusion of the sizing key.

7. The crimping device of claim 6, wherein the release button includes a first extension in contact with the pivoting member, and a second extension in contact with the fixed member.

8. The crimping device of claim 7, wherein the pivoting member is configured to rotate about a first rotational axis, and a first biasing member that is contact with both the pivoting member and the fixed member biases the pivoting member to rotate in a first direction about the first rotational axis.

9. The crimping device of claim 8, wherein upon being depressed, the release button causes the pivoting member to rotate in a second direction opposite the first direction about the first rotational axis while compressing the first biasing member.

10. The crimping device of claim 9, further comprising a second biasing member in contact with both the second extension of the release button and the fixing member, the second biasing member configured to move the release button from the depressed condition to a non-depressed condition.

11. The crimping device of claim 1, wherein the rotating body include a first shell coupled to a second shell.

12. The crimping device of claim 11, wherein the handle assembly includes a first arm and a second arm, the first arm being attached to the first shell and the second arm being attached to the second shell.

13. The crimping device of claim 11, wherein the handle assembly includes a first arm and a second arm, the first arm being integral with the first shell, the second arm being integral with the second shell.

14. The crimping device of claim 13, wherein the first arm and the second arm each have a free end, and a grip member extends between, and is coupled to, the free end of the first arm and the free end of the second arm.

15. The crimping device of claim 1, wherein the handle assembly is rotatable about a handle axis that is coaxial with the iris assembly.

16. The crimping device of claim 1, further comprising a retainer configured to be releasably coupled to the housing assembly, the retainer including a groove to receive a catheter of a delivery device, the groove being coaxial with the iris assembly when the retainer is coupled to the housing assembly.

17. A method of crimping a prosthetic heart valve, the method comprising:
attaching a sizing key to a rotating body of a crimper device;
positioning the prosthetic heart valve within an open iris assembly of the crimper device;
rotating a handle of the crimper device in a first direction to close the iris assembly to a prescribed minimum size over the prosthetic heart valve, until the sizing key abuts a stop surface of a holding member of the crimping device to prevent further rotation of the handle in the first direction;
waiting a prescribed amount of time while the iris assembly has the prescribed minimum size;
after waiting the prescribed amount of time, rotating the handle of the crimper device in a second direction opposite the first direction to open the iris assembly; and
removing the crimped prosthetic heart valve from the iris assembly.

18. The method of claim 17, wherein when the iris assembly has the prescribed minimum size, the sizing key abuts a holding surface of the holding member to prevent rotation of the handle in the second direction.

19. The method of claim 18, further comprising depressing a release button of the holding member after waiting the prescribed amount of time and prior to rotating the handle of the crimper device in the second direction.

20. The method of claim 17, wherein prior to attaching the sizing key to the rotating body, the rotating key is removed from packaging containing (i) the prosthetic heart valve and/or (ii) a delivery device for delivering the prosthetic heart valve.
